(19) United States
(12) Patent Application Publication
(10) Pub. No.: US 2007/0073039 A1
(43) Pub. Date: Mar. 29, 2007

(54) Peptides that inhibit viral infections

(76) Inventor: Francis V. Chisari, Del Mar, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/541,488

(22) Filed: Sep. 29, 2006

Related U.S. Application Data
(60) Provisional application No. 60/722,502, filed on Sep. 29, 2005.
(60) Provisional application No. 60/840,328, filed on Aug. 25, 2006.

(51) Int. Cl. A61K 31/70; C07K 14/00; A01N 43/04; C07K 16/00; A61K 38/00; C07K 17/00; C07K 2/00; C07K 4/00; C07K 5/00; C07K 7/00

(52) U.S. Cl. 530/300; 514/44

(57) ABSTRACT

The present application is directed to peptides that inhibit infection of a virus from the Flaviviridae family, methods of using these peptides to inhibit viral infections, and pharmaceutical compositions and combinations, as well as articles of manufacture comprising these peptides.

### RELATED APPLICATIONS

This application claims the benefit of US. Provisional Application Ser. No. 60/722,502 filed Sep. 29, 2005 and U.S. Provisional Application Ser. No. 60/840,328 filed Aug. 25, 2006, which applications are incorporated herein by reference.

### GOVERNMENT FUNDING

The invention described herein was made with United States Government support under Grant Number CA108304 awarded by the National Institutes of Health. The United States Government has certain rights in this invention.

### BACKGROUND OF THE INVENTION

Viral diseases can be very difficult to treat because viruses enter mammalian cells, where they perform many of their functions, including transcription and translation of viral proteins, as well as replication of the viral genome. Thus, viruses are protected not only from the host's immune system, but also from medicines administered to the host, as the viral infection progresses.

Thus, few effective anti-viral agents are currently available and most of those are effective against only a small subset of viruses. For example, researchers developed the first antiviral drug in the late 20th century and that drug, acyclovir, was approved by the U.S. Food and Drug Administration to treat herpes simplex virus infections. To date, only a few other antiviral medicines are available to prevent and/or treat viral infections.

Therefore, agents for treating and preventing viral infections are needed.

### SUMMARY OF THE INVENTION

The invention relates to peptides that inhibit infection of a virus of the Flaviviridae family. Surprisingly, many of the present peptides can act on viruses that are free in solution, and inhibit the virus before it has a chance to infect mammalian cells. One aspect of the invention relates to the discovery that peptides derived from the Hepatitis C polyprotein, e.g. those having sequences set forth in SEQ ID NO: 4-61, can inhibit infection from other viruses of the Flaviviridae family.

In one embodiment, the invention provides for an isolated peptide of 14 to 50 D- or L-amino acids in-length, having an amphipathic α-helical structure and anti-viral activity against a virus of the Flaviviridae family.

In one embodiment, the peptide has a sequence comprising any one of formulae I-V:

I

| | |
|---|---|
| Xaa₁-Xaa₂-Xaa₃-Xaa₄-Xaa₅-Xaa₆- | (SEQ ID NO: 112) |
| Xaa₇-Xaa₈-Xaa₉-Xaa₁₀-Xaa₁₁-Xaa₁₂- | |
| Xaa₁₃-Xaa₁₄ | |
| II | |
| Xaa₁-Xaa₂-Xaa₃-Xaa₄-Xaa₅-Xaa₆- | (SEQ ID NO: 113) |
| Xaa₇-Xaa₈-Xaa₉-Xaa₁₀-Xaa₁₁-Xaa₁₂- | |
| Xaa₁₃-Xaa₁₄-Xaa₁₅ | |
| III | |
| Xaa₁-Xaa₂-Xaa₃-Xaa₄-Xaa₅-Xaa₆- | (SEQ ID NO: 114) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆ | |
| IV | |
| Xaa₁-Xaa₂-Xaa₃-Xaa₄-Xaa₅-Xaa₆- | (SEQ ID NO: 115) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇ | |
| V | |
| Xaa₁-Xaa₂-Xaa₃-Xaa₄-Xaa₅-Xaa₆- | (SEQ ID NO: 116) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇- | |
| Xaa₁₈ | |

wherein: Xaa₁, Xaa₄, Xaa₅, Xaa₈, Xaa₁₁, Xaa₁₂, Xaa₁₅, Xaa₁₆ and Xaa₁₈ are separately each a polar amino acid; and Xaa₂, Xaa₃, Xaa₆, Xaa₇, Xaa₉, Xaa₁₀, Xaa₁₃, Xaa₁₄, and Xaa₁₇ are separately each a nonpolar amino acid.

In another embodiment, the invention provides a fusion peptide formed by attaching a 14 amino acid peptide (the N-terminyl peptide) to the N-terminus of a peptide of any of formulae I to V. The 14 amino acid N-terminyl peptide has the structure: Rx-Ry-Ry-Rx-Ry-Ry-Rx-Rx-Ry-Ry-Rx-Rx-Ry-Rx (SEQ ID NO: 117), wherein each Rx is separately a polar amino acid, and each Ry is separately a nonpolar amino acid.

In another embodiment, the invention provides a fusion peptide formed by attaching a 12 amino acid peptide (the C-terminyl peptide) to the C-terminus of a peptide of formula V. The resulting fusion peptide has the structure of formulae VI:

VI

| | |
|---|---|
| Xaa₁-Xaa₂-Xaa₃-Xaa₄-Xaa₅-Xaa₆- | (SEQ ID NO: 118) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇- | |
| Xaa₁₈-Xaa₁₉-Xaa₂₀-Xaa₂₁-Xaa₂₂- | |
| Xaa₂₃-Xaa₂₄-Xaa₂₅-Xaa₂₆-Xaa₂₇- | |
| Xaa₂₈-Xaa₂₉-Xaa₃₀, | |

wherein:

Xaa₁, Xaa₄, Xaa₅, Xaa₈, Xaa₁₁, Xaa₁₂, Xaa,₁₅, Xaa₁₆, Xaa₁₈, Xaa₁₉, Xaa₂₂, Xaa₂₃, Xaa₂₆, Xaa₂₉, and Xaa₃₀ are separately each a polar amino acid; and

Xaa₂, Xaa₃, Xaa₆, Xaa₇, Xaa₉, Xaa₁₀, Xaa₁₃, Xaa₁₄, Xaa₁₇, Xaa₂₀, Xaa₂₁, Xaa₂₄, Xaa₂₅, Xaa₂₇, and Xaa₂₈ are separately each a nonpolar amino acid.

In some embodiments, the invention provides a fusion peptide having a sequence that corresponds to the 14 amino acid N-terminyl peptide of SEQ ID NO: 117 attached by a peptide bond to the N-terminus of a peptide of formula VI.

In another embodiment, a peptide of the invention is a peptide comprising at least 14 contiguous amino acids of any of the above described peptides.

In some embodiments, nonpolar amino acids are selected from the group consisting of (1) alanine, valine, leucine, methionine, isoleucine, phenylalanine, and tryptophan or (2) valine, leucine, isoleucine, phenylalanine and tryptophan. In some embodiments, the polar amino acids are selected from the group consisting of (1) arginine, asparagine, aspartic acid, cysteine, glutamic acid, glutamine, histidine, homocysteine, lysine, hydroxylysine, ornithine, serine and threonine; or (2) arginine, aspartic acid, glutamic acid, cysteine and lysine.

In another embodiment, a peptide of the invention has an amino acid composition that consists of arginine, cysteine, glutamate, serine, valine, two aspartates, two leucines, two isoleucines and three tryptophan residues. For example, the peptide has an amino acid sequence of SEQ ID NO: 92 or 102.

In another embodiment, a peptide of the invention has an amino acid composition that consists of arginine, cysteine, glutamate, two serines, valine, two aspartates, two leucines, two isoleucines and three tryptophan residues. For example, the peptide has an amino acid sequence of SEQ ID NO: 93 or 101.

In another embodiment, a peptide of the invention has an amino acid composition that consists of arginine, cysteine, glutamate, two serines, valine, three aspartates, two leucines, two isoleucines and three tryptophan residues. For example, the peptide has an amino acid sequence of SEQ ID NO: 94 or 100.

In another embodiment, a peptide of the invention has an amino acid composition that consists of the residues arginine, cysteine, glutamate, two serines, valine, three aspartates, two leucines, two isoleucines, three tryptophan and a phenylalamine. For example, the peptide has an amino acid sequence of SEQ ID NO: 95 or 99.

In another embodiment, a peptide of the invention has an amino acid composition that consists of the residues arginine, cysteine, glutamate, two serines, valine, three aspartates, two leucines, two isoleucines, three tryptophan, a phenylalamine and a lysine. For example, the peptide has an amino acid sequence of SEQ ID NO: 43 and 96-98.

In another embodiment, the invention provides a peptide that comprises an amino acid sequence selected from the group consisting of SEQ ID NO: 43 and 91-102. In some embodiment, the peptide is 14 to 50 D- or L-amino acids in-length, comprises an amino acid sequence selected from the group consisting of SEQ ID NO: 43 and 91-102, and peptide has an amphipathic α-helical structure.

In another embodiment, the invention provides a peptide having the amino acid sequence of any of SEQ ID NO: 4-86. For example, the peptide has the amino acid sequence of any one of SEQ ID NO: 6, 8, 12, 13, 14, 21, 23, 24, 27, 28, 30, 32, 37, 44, 47, 48 and 53.

In some embodiments, a peptide of the invention includes D-amino acids. In other embodiments, a peptide of the invention includes L-amino acids. In some embodiments, the peptide includes a dansyl moiety. In some embodiments, the peptide has an EC₅₀ of about 500 nM or less; about 400 nM or less; or about 300 nM. In some embodiments, the peptides are active against a Hepatitis C virus or a Flavivirus such as the West Nile virus or the Dengue virus.

In another embodiment, the invention provides a pharmaceutical composition comprising any of the peptides of the invention discussed above. In some embodiments, the composition is a microbicide or a vaginal cream.

In another embodiment, the invention provides a pharmaceutical combination comprising any of the peptides of the invention discussed above and an antiviral agent such as α-interferon, pegylated interferon, ribavirin, amantadine, rimantadine, pleconaril, acyclovir, zidovudine, lamivudine, or a combination thereof.

In another embodiment, the invention provides a method for preventing viral infection in a mammalian cell that involves contacting the cell with any one or more of the peptides of the invention discussed above, as well as pharmaceutical compositions, or combinations, that include one or more of such peptides. In some embodiment, the mammalian cell is a human cell. In some embodiment, the virus is Hepatitis C virus or a Flavivirus such as West Nile virus or Dengue virus.

In another embodiment, the invention provides a method for preventing viral infection in a mammal that involve administering to the mammal an effective amount of any of the peptides and pharmaceutical compositions or combinations discussed above. In some embodiments, the mammal is a human. In some embodiments, the virus is a Flavivirus such as West Nile virus or Dengue virus or a Hepatitis C virus.

In another embodiment, the invention provides an article of manufacture comprising a vessel for collecting a body fluid and any one or more of the peptides of the invention discussed above. In some embodiments, the vessel is a collection bag, tube, capillary tube or syringe. In some embodiments, the vessel is evacuated. In some embodiments, the article also includes a biological stabilizer such as an anti-coagulant, preservative, protease inhibitor, or any combination thereof. In some embodiments, the anti-coagulant is citrate, ethylene diamine tetraacetic acid, heparin, oxalate, fluoride or any combination thereof. In some embodiments, the preservative is boric acid, sodium formate and sodium borate. In some embodiments, the protease inhibitor is dipeptidyl peptidase IV. In some embodiments, the peptide and/or stabilizer are freeze dried. In some embodiments, the peptide is attached or adsorbed onto the vessel so that the peptide is retained in the vessel after materials placed therein have been removed. When attached or adsorbed onto the vessel, the peptide is still able to inhibit viral infection.

In another embodiment, the invention provides a composition comprising a sample from the body of a mammal and any one or more of the peptides discussed above. In some embodiments, the composition further includes a biological stabilizer, which in some embodiments is an anti-coagulant, a preservative, a protease inhibitor, or any combination thereof. In some embodiments, the anticoagulant is citrate, ethylene diamine tetraacetic acid, heparin, oxalate, fluoride or any combination thereof. In some embodiments, the preservative is boric acid, sodium formate and sodium borate. In some embodiments, the protease inhibitor is dipeptidyl peptidase IV. In some embodiments, the sample is a blood product such as, without limitation, plasma, platelet, leukocytes or stem cell.

Other features and advantages of the invention will be apparent from the following detailed description, and from the claims.

### DESCRIPTION OF THE FIGURES

FIG. 1A illustrates that infectious hepatitis C virions are produced following transfection with genomic JFH-1 RNA. Intracellular RNA amplification was used to detect production of JFH-1 RNA. Ten micrograms of in vitro transcribed JFH-1 RNA was electroporated into 4×10⁶ Huh-7.5. 1 cells. Transfected cells and supernatant were harvested at the indicated days post-transfection. Total cellular RNA was analyzed for JFH-1 expression by real-time quantitative RT-PCR and displayed as genome equivalents/μg total RNA (line). Supernatant infectivity titers were determined on naive Huh-7.5.1 cells and shown as focus-forming units (ffu) per mL (bars).

FIG. 1B further confirms that the JFH-1 viral genome was actively replicating after transfection, in vitro transcribed wild type (wt) and polymerase mutant (GND) JFH-1 full length genomic RNA was electroporated into Huh-7.5.1 cells. Intracellular HCV RNA was monitored at different time points thereafter. As shown, the wild type viral RNA increased slightly from day 1 to day 2, followed by a 10-fold decrease on day 4. Intracellular HCV RNA levels then rebounded to above 10⁷ copies/μg total cellular RNA and were maintained for the remainder of the experiment. In contrast, intracellular levels of polymerase-deficient mutant JFH/GND RNA decayed rapidly after transfection by several orders of magnitude and became undetectable by day 20. These results indicate that wild type JFH-1 RNA was actively replicating in Huh-7.5.1 cells.

FIG. 1C illustrates the kinetics of HCV replication and generation of infectious virus after lipofectamin transfection of genomic JFH-1 RNA into Huh-7.5.1 cells. Huh-7.5.1 cells were transfected with JFH clone RNA by lipofection and cells and supernatants were periodically collected to analyze intracellular HCV RNA and infectivity titer in the supernatant, respectively. The graph represents HCV RNA accumulation as GE/μg of total RNA (lines) and virus titer in ffu/mL (bars) in the supernatant.

FIG. 2A-D illustrate detection of infected cells following transfection with genomic JFH-1 RNA. HCV infection was detected by cytoimmunofluorescence of the HCV NS5A protein. FIG. 2A shows expression of NS5A at 5 days post-transfection. FIG. 2B shows expression of NS5A at 24 days post-transfection. FIG. 2C shows expression of NS5A in naïve cells after exposure to undiluted supernatant collected from JFH-1 RNA transfected Huh-7.5.1 cells. FIG. 2D shows expression of NS5A in naïve cells after exposure to a 1:10 dilution of supernatant collected from JFH-1 RNA transfected Huh-7.5.1 cells. NS5A-positive cells were detected as red in the original (appearing as lighter bright spots in some copies of the original). Cell nuclei were stained with Hoescht dye (blue in the original, darker spots in copies).

FIG. 3A-D illustrate HCV infection kinetics and passage in tissue culture cells. Naïve Huh 7.5.1 cells were inoculated with culture supernatants at an MOI of 0.01. Supernatants from the inoculated cells were collected at the indicated times post-infection and evaluated for infectivity (ffu/mL). Data represent the average of two or more experiments with error bars. FIG. 3A shows the infectivity titer of Huh-7.5.1 cells inoculated with supernatant harvested at day 19 after transfection of Huh-7.5.1 cells with JFH-1 genomic RNA by electroporation (circular symbols) or day 24 after lipofection (diamond symbols). The x-axis shows the time in days after supernatant inoculation. FIG. 3B shows the infectivity titer of Huh-7.5.1 cells inoculated with supernatant collected at day 5 from the infection illustrated by the diamond symbols in FIG. 3A. FIG. 3C-D shows that NS5A immunostaining increases in Huh-7.5.1 cells at days 5 (FIG. 3C) and 7 (FIG. 3D) post-infection, when using the supernatant collected at day 5 from the infection whose data are shown in FIG. 3A (diamond symbols).

FIG. 3E-F further illustrate viral RNA and protein production during HCV infection. Huh-7.5.1 cells were infected at an MOI of 0.01, and cell extracts were prepared at the designated time points for RNA and protein analysis. FIG. 3E graphically illustrates the amounts of intracellular HCV RNA (line) and the infectivity titer of the supernatant (bars). FIG. 3F is an image of a Western Blot of electrophoretically-separated cellular proteins. As show, intracellular HCV core and NS3 proteins accumulated during as the infection progressed.

FIG. 3G is a graph indicating that HCV virus produced in cell supernatants can be serially passaged through naïve Huh-7 cells.

FIG. 4A-B illustrate that HCV infection is inhibited by anti-E2 and anti-CD81 antibodies. FIG. 4A shows the effects of anti-E2 antibodies. JFH-1 virus was pre-incubated with the indicated concentrations of anti-E2 antibody or irrelevant human IgG1 antibody for 1 hour at 37° C. before being used to inoculate Huh-7.5.1. cells. Total cellular RNA was analyzed by quantitative RT-PCR at day 3 post-infection. FIG. 4B shows the effects of anti-CD81 antibodies. Huh-7.5.1 cells were preincubated with the indicated concentrations of anti-human CD81 or control mouse IgG1 antibody for 1 hour at 37° C. before inoculation with JFH-1 virus at an MOI of 0.3. Total cellular RNA was analyzed by quantitative RT-PCR at day 3 post-infection.

FIG. 5 shows sucrose gradient sedimentation of infectious HCV. Supernatant from infected Huh-7.5.1 cells was fractionated as described in Example 1. Fractions (1-9) were collected from the top of the gradient and analyzed by quantitative RT-PCR for HCV RNA (line). The infectivity of each fraction was determined (bars) by titration. Fraction densities are expressed as g/mL.

FIG. 6 illustrates the kinetics of JFH-1 HCV infection in Huh-7.5.1 and Huh-7 cells. A virus stock generated in Huh-7.5.1 was diluted to infect Huh-7.5.1 and Huh-7 cells at an MOI of 0.01. Culture supernatant was collected at the indicated times and titrated. Infectious titers in Huh-7.5.1 (solid lines) and Huh-7 cells (dashed lines) are expressed as ffu/mL. Average values of two independent infection experiments are shown.

FIG. 7 illustrates that intracellular HCV RNA accumulates in Huh-7.5.1 and Huh-7 infected cells. Total RNA was isolated from the infected Huh-7.5.1 and Huh-7 cells described in FIG. 6. Intracellular HCV RNA accumulation in infected Huh-7.5.1 (solid lines) and Huh-7 (dashed lines) was determined by quantitative RT-PCR. The results are shown as the average genome equivalents (GE)/μg of total RNA of two independent infections (n=2).

FIG. 8 graphically illustrates inhibition of HCV infection by interferons. Forty-five thousand Huh-7.5.1 cells were plated and treated with 5, 50 and 500 IU/mL of human IFNα-2a and IFNγ for 6 hours, and then inoculated with recombinant JFH-1 virus at an MOI of 0.3 in the presence of the same doses of IFN. The viral inoculum was removed 4 hours later and the cells were further cultured with interferon for 3 days. At that time cells were harvested, RNA was isolated and analyzed by real-time RT-PCR to determine the intracellular HCV RNA levels. Bars represent intracellular HCV RNA levels expressed as a % of the levels obtained in the control infections. The results demonstrate that both interferons efficiently inhibit HCV infection.

FIG. 9 illustrates the location of the peptides with respect to the HCV polyprotein genotype 1a (H77 isolate, having SEQ ID NO:1) and the corresponding anti-HCV activity. Thirteen of the peptides tested inhibited infectivity by 90% or more.

FIG. 10A-D are graphically illustrate that peptide 1 having the sequence SWLRDIWDWICEVLSDFK (SEQ ID NO: 43) permanently prevents HCV infection when it was added to cells together with HCV (FIG. 10A) and abolishes ongoing HCV infection (FIG. 10B) with an EC₅₀ of 300 nM (FIG. 10C and D).

FIG. 11A-E are results showing inhibition of HCV attachment to Huh-7.5.1 cells by various synthetic peptides (FIG. 11A); a peptide is most effective when it is added together with the virus (“CO”) to the target cells than when pre-incubated (“PRE”) with the cells before adding virus or when added after the cells have been exposed to the virus (“POST”) (FIG. 11B); preincubation of virus with peptide 1 completely abolishes viral infectivity (FIG. 11C); preincubation of virus with peptide 1 reduces the total viral RNA content by at least 3-fold indicating viral lysis (FIG. 11D, where the left panel shows HCV RNA and the right panel shows GAPDH RNA); preincubation of virus with peptide completely abolishes infectivity and reduces the viral RNA content of all fractions by approximately 4-5 fold (E).

FIG. 12A-C are results showing that the D-form of peptide 1 is fully active and displays enhanced serum stability (A), and that the EC₅₀ of the L- and D-forms of peptide 1 are very similar (B and C, respectively), where both are in the 1 μM range.

FIG. 13A-B are results showing the toxicity (LD₅₀) of the L- and D-forms of peptide 1 on Huh-7, Huh-7.5.1, HeLa and HepG2 cells (A); and the hemolytic activity of the L- and D-form of peptide 1 (B).

FIG. 14A-E illustrate the amphipathic α-helical nature of peptide 1 (SEQ ID NO:43). Helical wheel diagram of peptide 1 shows that the amino acid distribution results in a hydrophilic (or polar) face and a hydrophobic (or non-polar) face (FIG. 14A). Circular dichroism results show the α-helical structure of the L- and D-isomers of peptide 1 (FIG. 14B), the effect of dansylation on the α-helical structure of the L- and D-isomers of peptide 1 (FIG. 14C), and the α-helical structures of variants of peptide 1 having C-terminal truncations (FIG. 14D) and N-terminal truncations (FIG. 14E). The sequences of these truncated peptides are provided in Table 7.

FIG. 15A-B illustrate the liposome-release assays in general (A) and the results obtained for various truncation variants of peptide 1 (B). The sequences of these truncated peptides are provided in Table 7.

FIG. 16 is a graph showing that peptide 1 does not block vesicular stomatitis virus (VSV) infection.

FIG. 17 is a graph showing that peptide 2022 (peptide 1) with sequence SWLRDIWDWICEVLSDFK (SEQ ID NO:43) and peptide 2013 having the sequence SWLRDIWDWICEVL (SEQ ID NO:92) inhibit essentially 100% of Dengue viral infection as detected by ELISA. Peptide 2017 having the sequence LRDIWDWICEVLSDFK (SEQ ID NO:107) had slightly less activity, inhibiting Dengue viral infection by about 84%.

FIG. 18 is a graph showing dose-dependent inhibition of Dengue viral infection by peptide 2022 (peptide 1), peptide 2013, and peptide 2017, as detected by FACS analysis of cells intracellularly stained for Dengue viral antigens. As shown, at concentrations of 20 μM almost 100% of Dengue viral infection was inhibited by peptide 2022 (peptide 1) and peptide 2013, as detected by FACS. Peptide 2017 at 20 μM had slightly less activity, inhibiting Dengue viral infection by about 80%.

FIG. 19 is a graph showing that peptide 2022 (peptide 1) inhibits essentially 100% of Dengue viral infection as detected by an immunofluorescence assay. Peptide 2017 had slightly less activity, inhibiting Dengue viral infection by about 90%.

FIG. 20 is data illustrating the effectiveness of peptide 1 in inhibiting West Nile viral infection.

### DETAILED DESCRIPTION OF THE INVENTION

The invention relates to peptides that inhibit viral infection. The invention involves the discovery that certain peptides derived from the HCV polyprotein, e.g. those having sequences set out in SEQ ID NO: 4-61, can inhibit infection of mammalian cells by virus of the Flaviviridae family. The invention also involves the discovery of thirteen peptides from the HCV polyprotein (SEQ ID NO:1) that are highly effective at inhibiting HCV infection. In addition, the invention involves the discovery that “peptide 1” (SEQ ID NO:43), derived from the membrane anchor domain of NS5A (NS5A-1975), was particularly potent against HCV, as well as against Flaviviruses such as the Dengue virus and the West Nile virus. For example, a single dose of peptide 1 completely blocked HCV infection with an EC₅₀ of 289 nM without evidence of cytotoxicity. In addition, 20 μM of peptide 1 completely inhibited Dengue viral infection.

Accordingly, the invention provides peptides that are effective at inhibiting infection by one or more viruses of the Flaviviridae family. Peptides of the invention include, for example, those having sequences set out in SEQ ID NO: 4-61, 91-102, and peptides of about 8 to about 50 amino acids that are capable of forming an α-helical structure and can inhibit viral infection in a mammalian cell. The invention provides an antiviral peptide or combinations of antiviral peptides, various compositions and combinations containing such antiviral peptide(s), and a method for inhibiting viral infection in a mammalian cell that utilizes such peptide(s). The invention also provides an article of manufacture containing such antiviral peptide(s).

Hepatitis C Virus

Hepatitis C virus (HCV) is a noncytopathic, positive-stranded RNA virus that causes acute and chronic hepatitis and hepatocellular carcinoma. Hooffiagle, J. H. (2002) Hepatology 36, S21-29. The hepatocyte is the primary target cell, although various lymphoid populations, especially B cells and dendritic cells may also be infected at lower levels. Kanto et al. (1999) J. Immunol. 162, 5584-5591; Auffermann-Gretzinger et al. (2001) Blood 97, 3171-3176; Hiasa et al. (1998) Biochem. Biophys. Res. Commun. 249, 90-95. A striking feature of HCV infection is its tendency towards chronicity with at least 70% of acute infections progressing to persistence (Hoofnagle, J. H. (2002) Hepatology 36, S21-29). HCV chronicity is often associated with significant liver disease, including chronic active hepatitis, cirrhosis and hepatocellular carcinoma (Alter, H. J. & Seeff, L. B. (2000) Semin. Liver Dis. 20, 17-35). Thus, with over 170 million people currently infected (id.), HCV represents a growing public health concern.

The single stranded HCV RNA genome is approximately 9500 nucleotides in length and has a single open reading frame (ORF) encoding a large polyprotein. The polyprotein has about 3010-3033 amino acids (Q.-L. Choo, et al. Proc. Natl. Acad. Sci. USA 88, 2451-2455 (1991); N. Kato et al., Proc. Natl. Acad. Sci. USA 87, 9524-9528 (1990); A. Takamizawa et al., J. Virol. 65,1105-1113 (1991)).

Nucleic acid and amino acid sequences for different isolates of HCV can be found in the art, for example, in the NCBI database. See ncbi.nlm.nih.gov. An example of an HCV polyprotein sequence can be found in the NCBI database as accession number NP 671491 (gi: 22129793). The amino acid sequence of NP 671491 (SEQ ID NO:1) is as follows.

| | |
|---|---|
| 1 | MSTNPKPQRK TKRNTNRRPQ DVKFPGGGQI VGGVYLLPRR |
| 41 | GPRLGVRATR KTSERSQPRG RRQPIPKARR PEGRTWAQPG |
| 81 | YPWPLYGNEG CGWAGWLLSP RGSRPSWGPT DPRRRSRNLG |
| 121 | KVIDTLTCGF ADLMGYIPLV GAPLGGAARA LAHGVRVLED |
| 161 | GVNYATGNLP GCSFSIFLLA LLSCLTVPAS AYQVRNSSGL |
| 201 | YHVTNDCPNS SIVYEAADAI LHTPGCVPCV REGNASRCWV |
| 241 | AVTPTVATRD GKLPTTQLRR HIDLLVGSAT LCSALYVGDL |
| 281 | CGSVFLVGQL FTFSPRRHWT TQDCNCSIYP GHITGHRMAW |
| 321 | DMMMNWSPTA ALVVAQLLRI PQAIMDMIAG AHWGVLAGIA |
| 361 | YFSMVGNWAK VLVVLLLFAG VDAETHVTGG SAGRTTAGLV |
| 401 | GLLTPGAKQN IQLINTNGSW HINSTALNCN ESLNTGWLAG |
| 441 | LFYQHKFNSS GCPERLASCR RLTDFAQGWG PISYANGSGL |
| 481 | DERPYCWHYP PRPCGIVPAK SVCGPVYCFT PSPVVVGTTD |
| 521 | RSGAPTYSWG ANDTDVFVLN NTRPPLGNWF GCTWMNSTGF |
| 561 | TKVCGAPPCV IGGVGNNTLL CPTDCFRKHP EATYSRCGSG |
| 601 | PWITPRCMVD YPYRLWHYPC TINYTIFKVR MYVGGVEHRL |
| 641 | EAACNWTRGE RCDLEDRDRS ELSPLLLSTT QWQVLPCSFT |
| 681 | TLPALSTGLI HLHQNIVDVQ YLYGVGSSIA SWAIKWEYVV |
| 721 | LLFLLLADAR VCSCLWMMLL ISQAEAALEN LVILNAASLA |
| 761 | GTHGLVSFLV FFCFAWYLKG RWVPGAVYAF YGMWPLLLLL |
| 801 | LALPQRAYAL DTEVAASCGG VVLVGLMALT LSPYYKRYIS |
| 841 | WCMWWLQYFL TRVEAQLHVW VPPLNVRGGR DAVILLMCVV |
| 881 | HPTLVFDITK LLLAIFGPLW ILQASLLKVP YFVRVQGLLR |
| 921 | ICALARKIAG GHYVQMAIIK LGALTGTYVY NHLTPLRDWA |
| 961 | HNGLRDLAVA VEPVVFSRME TKLITWGADT AACGDIINGL |
| 1001 | PVSARRGQEI LLGPADGMVS KGWRLLAPIT AYAQQTRGLL |
| 1041 | GCIITSLTGR DKNQVEGEVQ TVSTATQTFL ATCINGVCWT |
| 1081 | VYHGAGTRTI ASPKGPVIQM YTNVDQDLVG WPAPQGSRSL |
| 1121 | TPCTCGSSDL YLVTRHADVI PVRRRGDSRG SLLSPRPISY |
| 1161 | LKGSSGGPLL CPAGHAVGLF RAAVCTRGVA KAVDFIPVEN |
| 1201 | LETTMRSPVF TDNSSPPAVP QSFQVAHLHA PTGSGKSTKV |
| 1241 | PAAYAAQGYK VLVLNPSVAA TLGFGAYMSK AHGVDPNIRT |
| 1281 | GVRTITTGSP ITYSTYGKFL ADGGCSGGAY DIIICDECHS |
| 1321 | TDATSILGIG TVLDQAETAG ARLVVLATAT PPGSVTVSHP |
| 1361 | NIEEVALSTT GEIPFYGKAI PLEVIKGGRH LIFCHSKKKC |
| 1401 | DELAAKLVAL GINAVAYYRG LDVSVIPTSG DVVVVSTDAL |
| 1441 | MTGFTGDFDS VIDCNTCVTQ TVDFSLDPTF TIETTTLPQD |
| 1481 | AVSRTQRRGR TGRGKPGIYR FVAPGERPSG MFDSSVLCEC |
| 1521 | YDAGCAWYEL TPAETTVRLR AYMNTPGLPV CQDHLEFWEG |
| 1561 | VFTGLTHIDA HFLSQTKQSG ENFPYLVAYQ ATVCARAQAP |
| 1601 | PPSWDQMWKC LIRLKPTLHG PTPLLYRLGA VQNEVTLTHP |
| 1641 | ITKYIMTCMS ADLEVVTSTW VLVGGVLAAL AAYCLSTGCV |
| 1681 | VIVGRIVLSG KPAIIPDREV LYQEFDEMEE CSQHLPYIEQ |
| 1721 | GMMLAEQFKQ KALGLLQTAS RQAEVITPAV QTNWQKLEVF |
| 1761 | WAKHMWNFIS GIQYLAGLST LPGNPAIASL MAFTAAVTSP |
| 1801 | LTTGQTLLFN ILGGWVAAQL AAPGAATAFV GAGLAGAAIG |
| 1841 | SVGLGKVLVD ILAGYGAGVA GALVAFKIMS GEVPSTEDLV |
| 1881 | NLLPAILSPG ALVVGVVCAA ILRRHVGPGE GAVQWMNRLI |
| 1921 | AFASRGNHVS PTHYVPESDA AARVTAILSS LTVTQLLRRL |
| 1961 | HQWISSECTT PCSGSWLRDI WDWICEVLSD FKTWLKAKLM |
| 2001 | PQLPGIPFVS CQRGYRGVWR GDGIMHTRCH CGAEITGHVK |
| 2041 | NGTMRIVGPR TCRNMWSGTF PINAYTTGPC TPLPAPNYKF |
| 2081 | ALWRVSAEEY VEIRRVGDFH YVSGMTTDNL KCPCQIPSPE |
| 2121 | FFTELDGVRL HRFAPPCKPL LREEVSFRVG LHEYPVGSQL |
| 2161 | PCEPEPDVAV LTSMLTDPSH ITAEAAGRRL ARGSPPSMAS |
| 2201 | SSASQLSAPS LKATCTANHD SPDAELIEAN LLWRQEMGGN |
| 2241 | ITRVESENKV VILDSFDPLV AEEDEREVSV PAEILRKSRR |
| 2281 | FARALPVWAR PDYNPPLVET WKKPDYEPPV VHGCPLPPPR |
| 2321 | SPPVPPPRKK RTVVLTESTL STALAELATK SFGSSSTSGI |
| 2361 | TGDNTTTSSE PAPSGCPPDS DVESYSSMPP LEGEPGDPDL |
| 2401 | SDGSWSTVSS GADTEDVVCC SMSYSWTGAL VTPCAAEEQK |
| 2441 | LPINALSNSL LRHHNLVYST TSRSACQRQK KVTFDRLQVL |
| 2481 | DSHYQDVLKE VKAAASKVKA NLLSVEEACS LTPPHSAKSK |
| 2521 | FGYGAKDVRC HARKAVAHIN SVWKDLLEDS VTPIDTTIMA |
| 2561 | KNEVFCVQPE KGGRKPARLI VFPDLGVRVC EKMALYDVVS |
| 2601 | KLPLAVMGSS YGFQYSPGQR VEFLVQAWKS KKTPMGFSYD |
| 2641 | TRCFDSTVTE SDIRTEEAIY QCCDLDPQAR VAIKSLTERL |
| 2681 | YVGGPLTNSR GENCGYRRCR ASGVLTTSCG NTLTCYIKAR |
| 2721 | AACRAAGLQD CTMLVCGDDL VVICESAGVQ EDAASLRAFT |
| 2761 | EAMTRYSAPP GDPPQPEYDL ELITSCSSNV SVAHDGAGKR |
| 2801 | VYYLTRDPTT PLARAAWETA RHTPVNSWLG NIIMFAPTLW |
| 2841 | ARMILMTHFF SVLIARDQLE QALNCEIYGA CYSIEPLDLP |
| 2881 | PIIQRLHGLS AFSLHSYSPG EINRVAACLR KLGVPPLRAW |
| 2921 | RHRARSVRAR LLSRGGRAAI CGKYLFNWAV RTKLKLTPIA |
| 2961 | AAGRLDLSGW FTAGYSGGDI YHSVSHARPR WFWFCLLLLA |
| 3001 | AGVGIYLLPN R |

Another example of an HCV polyprotein amino acid sequence that can be found in the NCBI database is accession number BAB32872 (gi: 13122262). See ncbi.nlm.nih.gov; Kato et al. J. Med. Virol. 64: 334-339 (2001). This HCV was isolated from a fulminant hepatitis patient, and its amino acid sequence (SEQ ID NO:2) is as follows.

| | |
|---|---|
| 1 | MSTNPKPQRK TKRNTNRRPE DVKFPGGGQI VGGVYLLPRR |
| 41 | GPRLGVRTTR KTSERSQPRG RRQPIPKDRR STGKAWGKPG |
| 81 | RPWPLYGNEG LGWAGWLLSP RGSRPSWGPT DPRHRSRNVG |
| 121 | KVIDTLTCGF ADLMGYIPVV GAPLSGAARA VAHGVRVLED |
| 161 | GVNYATGNLP GFPFSIFLLA LLSCITVPVS AAQVKNTSSS |
| 201 | YMVTNDCSND SITWQLEAAV LHVPGCVPCE RVGNTSRCWV |
| 241 | PVSPNMAVRQ PGALTQGLRT HIDMVVMSAT FCSALYVGDL |
| 281 | CGGVMLAAQV FIVSPQYHWF VQECNCSIYP GTITGHRMAW |
| 321 | DMMMNWSPTA TMILAYVMRV PEVIIDIVSG AHWGVMFGLA |
| 361 | YFSMQGAWAK VIVILLLAAG VDAGTTTVGG AVARSTNVIA |
| 401 | GVFSHGPQQN TQLINTNGSW HINRTALNCN DSLNTGFLAA |
| 441 | LFYTNRFNSS GCPGRLSACR NIEAFRIGWG TLQYEDNVTN |
| 481 | PEDMRPYCWH YPPKPCGVVP ARSVCGPVYC ETPSPVVVGT |
| 521 | TDRRGVPTYT WGENETDVFL LNSTRPPQGS WFGCTWMNST |
| 561 | GFTKTCGAPP CRTRADFNAS TDLLCPTDCF RKHPDATYIK |
| 601 | CGSGPWLTPK CLVHYPYRLW HYPCTVNFTI FKIRMYVGGV |
| 641 | EHRLTAACNF TRGDRCDLED RDRSQLSPLL HSTTEWAILP |
| 681 | CTYSDLPALS TGLLHLHQNI VDVQYMYGLS PAITKYVVRW |
| 721 | EWVVLLFLLL ADARVCACLW MLILLGQAEA ALEKLVVLHA |
| 761 | ASAANCHGLL YFAIFFVAAW HIRGRVVPLT TYCLTGLWPF |
| 801 | CLLLMALPRQ AYAYDAPVHG QIGVGLLILI TLFTLTPGYK |
| 841 | TLLGQCLWWL CYLLTLGEAM IQEWVPPMQV RGGRDGIAWA |
| 881 | VTIFCPGVVF DITKWLLALL GPAYLLRAAL THVPYFVRAH |
| 921 | ALIRVCALVK QLAGGRYVQV ALLALGRWTG TYIYDHLTPM |
| 961 | SDWAASGLRD LAVAVEPIIF SPMEKKVIVW GAETAACGDI |
| 1001 | LHGLPVSARL GQEILLGPAD GYTSKGWKLL APITAYAQQT |
| 1041 | RGLLGAIVVS MTGRDRTEQA GEVQILSTVS QSFLGTTISG |
| 1081 | VLWTVYHGAG NKTLAGLRGP VTQMYSSAEG DLVGWPSPPG |
| 1121 | TKSLEPCKCG AVDLYLVTRN ADVIPARRRG DKRGALLSPR |
| 1161 | PISTLKGSSG GPVLCPRGHV VGLFRAAVCS RGVAKSIDFI |
| 1201 | PVETLDVVTR SPTFSDNSTP PAVPQTYQVG YLHAPTGSGK |
| 1241 | STKVPVAYAA QGYKVLVLNP SVAATLGFGA YLSKAHGINP |
| 1281 | NIRTGVRTVM TGEAITYSTY GKFLADGGCA SGAYDIIICD |
| 1321 | ECHAVDATSI LGIGTVLDQA ETAGVRLTVL ATATPPGSVT |
| 1361 | TPHPDIEEVG LGREGEIPFY GRAIPLSCIK GGRHLIFCHS |
| 1401 | KKKCDELAAA LRGMGLNAVA YYRGLDVSII PAQGDVVVVA |
| 1441 | TDALMTGYTG DFDSVIDCNV AVTQAVDFSL DPTFTITTQT |
| 1481 | VPQDAVSRSQ RRGRTGRGRQ GTYRYVSTGE RASGMFDSVV |
| 1521 | LCECYDAGAA WYDLTPAETT VRLRAYFNTP GLPVCQDHLE |
| 1561 | FWEAVFTGLT HIDAHFLSQT KQAGENFAYL VAYQATVCAR |
| 1601 | AKAPPPSWDA MWKCLARLKP TLAGPTPLLY RLGPITNEVT |
| 1641 | LTHPGTKYIA TCMQADLEVM TSTWVLAGGV LAAVAAYCLA |
| 1681 | TGCVSIIGRL HVNQRVVVAP DKEVLYEAFD EMEECASRAA |
| 1721 | LIEEGQRIAE MLKSKIQGLL QQASKQAQDI QPAMQASWPK |
| 1761 | VEQFWARHMW NFISGIQYLA GLSTLPGNPA VASMMAFSAA |
| 1801 | LTSPLSTSTT ILLNIMGGWL ASQIAPPAGA TGFVVSGLVG |
| 1841 | AAVGSIGLGK VLVDILAGYG AGISGALVAF KIMSGEKPSM |
| 1881 | EDVINLLPGI LSPGALVVGV ICAAILRRHV GPGEGAVQWM |
| 1921 | NRLIAFASRG NHVAPTHYVT ESDASQRVTQ LLGSLTITSL |
| 1961 | LRRLHNWITE DCPIPCSGSW LRDVWDWVCT ILTDFKNWLT |
| 2001 | SKLFPKLPGL PFISCQKGYK GVWAGTGIMT TRCPCGANIS |
| 2041 | GNVRLGSMRI TGPKTCMNTW QGTFPINCYT EGQCAPKPPT |
| 2081 | NYKTAIWRVA ASEYAEVTQH GSYSYVTGLT TDNLKIPCQL |
| 2121 | PSPEFFSWVD GVQIHRFAPT PKPFFRDEVS FCVGLNSYAV |
| 2161 | GSQLPCEPEP DADVLRSMLT DPPHITAETA ARRLARGSPP |
| 2201 | SEASSSVSQL SAPSLRATCT THSNTYDVDM VDANLLMEGG |
| 2241 | VAQTEPESRV PVLDFLEPMA EEESDLEPSI PSECMLPRSG |
| 2281 | FPRALPAWAR PDYNPPLVES WRRPDYQPPT VAGCALPPPK |
| 2321 | KAPTPPPRRR RTVGLSESTI SEALQQLAIK TFGQPPSSGD |
| 2361 | AGSSTGAGAA ESGGPTSPGE PAPSETGSAS SMPPLEGEPG |
| 2401 | DPDLESDQVE LQPPPQGGGV APGSGSGSWS TCSEEDDTTV |
| 2441 | CCSMSYSWTG ALITPCSPEE EKLPINPLSN SLLRYHNKVY |
| 2481 | CTTSKSASQR AKKVTFDRTQ VLDAHYDSVL KDIKLAASKV |
| 2521 | SARLLTLEEA CQLTPPHSAR SKYGFGAKEV RSLSGRAVNH |
| 2561 | IKSVWKDLLE DPQTPIPTTI MAKNEVFCVD PAKGGKKPAR |
| 2601 | LIVYPDLGVR VCEKMALYDI TQKLPQAVMG ASYGFQYSPA |
| 2641 | QRVEYLLKAW AEKKDPMGFS YDTRCFDSTV TERDIRTEES |
| 2681 | IYQACSLPEE ARTAIHSLTE RLYVGGPMFN SKGQTCGYRR |
| 2721 | CRASGVLTTS MGNTITCYVK ALAACKAAGI VAPTMLVCGD |
| 2761 | DLVVISESQG TEEDERNLRA FTEAMTRYSA PPGDPPRPEY |
| 2801 | DLELITSCSS NVSVALGPRG RRRYYLTRDP TTPLARAAWE |
| 2841 | TVRHSPINSW LGNIIQYAPT IWVRMVLMTH FFSILMVQDT |
| 2881 | LDQNLNFEMY GSVYSVNPLD LPAIIERLHG LDAFSMHTYS |
| 2921 | HHELTRVASA LRKLGAPPLR VWKSRARAVR ASLISRGGKA |
| 2961 | AVCGRYLFNW AVKTKLKLTP LPEARLLDLS SWFTVGAGGG |
| 3001 | DIFHSVSRAR PRSLLFGLLL LFVGVGLFLL PAR |

Another example of an HCV polyprotein amino acid sequence can be found in the NCBI database as accession number Q9WMX2 (gi: 68565847). See ncbi.nlm.nih.gov. This sequence was obtained from the Con1 isolate of HCV. The amino acid sequence (SEQ ID NO:3) is the following.

| | |
|---|---|
| 1 | MSTNPKPQRK TKRNTNRRPQ DVKFPGGGQI VGGVYLLPRR |
| 41 | GPRLGVRATR KTSERSQPRG RRQPIPKARQ PEGRAWAQPG |
| 81 | YPWPLYGNEG LGWAGWLLSP RGSRPSWGPT DPRRRSRNLG |
| 121 | KVIDTLTCGF ADLMGYIPLV GAPLGGAARA LAHGVRVLED |
| 161 | GVNYATGNLP GCSFSIFLLA LLSCLTIPAS AYEVRNVSGV |
| 201 | YHVTNDCSNA SIVYEAADMI MHTPGCVPCV RENNSSRCWV |
| 241 | ALTPTLAARN ASVPTTTIRR HVDLLVGAAA LCSAMYVGDL |
| 281 | CGSVFLVAQL FTFSPRRHET VQDCNCSIYP GHVTGHRMAW |
| 321 | DMMMNWSPTA ALVVSQLLRI PQAVVDMVAG AHWGVLAGLA |
| 361 | YYSMVGNWAK VLIVMLLFAG VDGGTYVTGG TMAKNTLGIT |
| 401 | SLFSPGSSQK IQLVNTNGSW HINRTALNCN DSLNTGFLAA |
| 441 | LFYVHKFNSS GCPERMASCS PIDAFAQGWG PITYNESHSS |
| 481 | DQRPYCWHYA PRPCGIVPAA QVCGPVYCFT PSPVVVGTTD |
| 521 | RFGVPTYSWG ENETDVLLLN NTRPPQGNWF GCTWMNSTGF |
| 561 | TKTCGGPPCN IGGIGNKTLT CPTDCFRKHP EATYTKCGSG |
| 601 | PWLTPRCLVH YPYRLWHYPC TVNFTIFKVR MYVGGVEHRL |
| 641 | EAACNWTRGE RCNLEDRDRS ELSPLLLSTT EWQVLPCSFT |
| 681 | TLPALSTGLI HLHQNVVDVQ YLYGIGSAVV SFAIKWEYVL |
| 721 | LLFLLLADAR VCACLWMMLL IAQAEAALEN LVVLNAASVA |
| 761 | GAHGILSFLV FFCAAWYIKG RLVPGAAYAL YGVWPLLLLL |
| 801 | LALPPRAYAM DREMAASCGG AVFVGLILLT LSPHYKLFLA |
| 841 | RLIWWLQYFI TRAEAHLQVW IPPLNVRGGR DAVILLTCAI |
| 881 | HPELIFTITK ILLAILGPLM VLQAGITKVP YFVRAHGLIR |
| 921 | ACMLVRKVAG GHYVQMALMK LAALTGTYVY DHLTPLRDWA |
| 961 | HAGLRDLAVA VEPVVFSDME TKVITWGADT AACGDIILGL |
| 1001 | PVSARRGREI HLGPADSLEG QGWRLLAPIT AYSQQTRGLL |
| 1041 | GCIITSLTGR DRNQVEGEVQ VVSTATQSFL ATCVNGVCWT |
| 1081 | VYHGAGSKTL AGPKGPITQM YTNVDQDLVG WQAPPGARSL |
| 1121 | TPCTCGSSDL YLVTRHADVI PVRRRGDSRG SLLSPRPVSY |
| 1161 | LKGSSGGPLL CPSGHAVGIF RAAVCTRGVA KAVDFVPVES |
| 1201 | METTMRSPVF TDNSSPPAVP QTFQVAHLHA PTGSGKSTKV |
| 1241 | PAAYAAQGYK VLVLNPSVAA TLGFGAYMSK AHGIDPNIRT |
| 1281 | GVRTITTGAP ITYSTYGKFL ADGGCSGGAY DIIICDECHS |
| 1321 | TDSTTILGIG TVLDQAETAG ARLVVLATAT PPGSVTVPHP |
| 1361 | NIEEVALSST GEIPFYGKAI PIETIKGGRH LIFCHSKKKC |
| 1401 | DELAAKLSGL GLNAVAYYRG LDVSVIPTSG DVIVVATDAL |
| 1441 | MTGFTGDFDS VIDCNTCVTQ TVDFSLDPTF TIETTTVPQD |
| 1481 | AVSRSQRRGR TGRGRMGIYR FVTPGERPSG MFDSSVLCEC |
| 1521 | YDAGCAWYEL TPAETSVRLR AYLNTPGLPV CQDHLEFWES |
| 1561 | VFTGLTHIDA HFLSQTKQAG DNFPYLVAYQ ATVCARAQAP |
| 1601 | PPSWDQMWKC LIRLKPTLHG PTPLLYRLGA VQNEVTTTHP |
| 1641 | ITKYIMACMS ADLEVVTSTW VLVGGVLAAL AAYCLTTGSV |
| 1681 | VIVGRIILSG KPAIIPDREV LYREFDEMEE CASHLPYIEQ |
| 1721 | GMQLAEQFKQ KAIGLLQTAT KQAEAAAPVV ESKWRTLEAF |
| 1761 | WAKHMWNFIS GIQYLAGLST LPGNPAIASL MAFTASITSP |
| 1801 | LTTQHTLLFN ILGGWVAAQL APPSAASAFV GAGIAGAAVG |
| 1841 | SIGLGKVLVD ILAGYGAGVA GALVAFKVMS GEMPSTEDLV |
| 1881 | NLLPAILSPG ALVVGVVCAA ILRRHVGPGE GAVQWMNRLI |
| 1921 | AFASRGNHVS PTHYVPESDA AARVTQILSS LTITQLLKRL |
| 1961 | HQWINEDCST PCSGSWLRDV WDWICTVLTD FKTWLQSKLL |
| 2001 | PRLPGVPFFS CQRGYKGVWR GDGIMQTTCP CGAQITGHVK |
| 2041 | NGSMRIVGPR TCSNTWHGTF PINAYTTGPC TPSPAPNYSR |
| 2081 | ALWRVAAEEY VEVTRVGDFH YVTGMTTDNV KCPCQVPAPE |
| 2121 | FFTEVDGVRL HRYAPACKPL LREEVTFLVG LNQYLVGSQL |
| 2161 | PCEPEPDVAV LTSMLTDPSH ITAETAKRRL ARGSPPSLAS |
| 2201 | SSASQLSAPS LKATCTTRHD SPDADLIEAN LLWRQEMGGN |
| 2241 | ITRVESENKV VILDSFEPLQ AEEDEREVSV PAEILRRSRK |
| 2281 | FPRAMPIWAR PDYNPPLLES WKDPDYVPPV VHGCPLPPAK |
| 2321 | APPIPPPRRK RTVVLSESTV SSALAELATK TFGSSESSAV |
| 2361 | DSGTATASPD QPSDDGDAGS DVESYSSMPP LEGEPGDPDL |
| 2401 | SDGSWSTVSE EASEDVVCCS MSYTWTGALI TPCAAEETKL |
| 2441 | PINALSNSLL RHHNLVYATT SRSASLRQKK VTFDRLQVLD |
| 2481 | DHYRDVLKEM KAKASTVKAK LLSVEEACKL TPPHSARSKF |
| 2521 | GYGAKDVRNL SSKAVNHIRS VWKDLLEDTE TPIDTTIMAK |
| 2561 | NEVFCVQPEK GGRKPARLIV FPDLGVRVCE KMALYDVVST |
| 2601 | LPQAVMGSSY GFQYSPGQRV EFLVNAWKAK KCPMGFAYDT |
| 2641 | RCFDSTVTEN DIRVEESIYQ CCDLAPEARQ AIRSLTERLY |
| 2681 | IGGPLTNSKG QNCGYRRCRA SGVLTTSCGN TLTCYLKAAA |
| 2721 | ACRAAKLQDC TMLVCCDDLV VICESAGTQE DEASLRAFTE |
| 2761 | AMTRYSAPPG DPPKPEYDLE LITSCSSNVS VAHDASGKRV |
| 2801 | YYLTRDPTTP LARAAWETAR HTPVNSWLGN IIMYAPTLWA |
| 2841 | RMILMTHFFS ILLAQEQLEK ALDCQIYGAC YSIEPLDLPQ |
| 2881 | IIQRLHGLSA FSLHSYSPGE INRVASCLRK LGVPPLRVWR |
| 2921 | HRARSVRARL LSQGGRAATC GKYLFNWAVR TKLKLTPIPA |
| 2961 | ASQLDLSSWF VAGYSGGDIY HSLSRARPRW FMWCLLLLSV |
| 3001 | GVGIYLLPNR |

Additional examples of HCV polyprotein sequences are available. For example a Taiwan isolate of hepatitis C virus is available in the NCBI database at accession number P29846 (gi: 266821). See ncbi.nlm.nih.gov.

In infected cells, the HCV polyprotein is cleaved at multiple sites by cellular and viral proteases to produce the structural and non-structural (NS) proteins. The generation of mature nonstructural proteins (NS2, NS3, NS4A, NS4B, NS5A, and NS5B) is affected by two viral proteases. The first one, as yet poorly characterized, cleaves at the NS2-NS3 junction; the second one is a serine protease contained within the N-terminal region of NS3 (henceforth referred to as NS3 protease) and mediates all the subsequent cleavages downstream of NS3, both in cis, at the NS3-NS4A cleavage site, and in trans, for the remaining NS4A-NS4B, NS4B-NS5A, NS5A-NS5B sites. The NS4A protein appears to serve multiple functions, acting as a cofactor for the NS3 protease and possibly assisting in the membrane localization of NS3 and other viral replicase components. The complex formation of the NS3 protease with NS4A seems necessary to the processing events, enhancing the proteolytic efficiency at all of the sites. The NS3 protein also exhibits nucleoside triphosphatase and RNA helicase activities. NS5B is a RNA-dependent RNA polymerase that is involved in the replication of HCV.

The HCV nonstructural (NS) proteins are presumed to provide the essential catalytic machinery for viral replication. The first 181 amino acids of NS3 (residues 1027-1207 of the viral polyprotein) have been shown to contain the serine protease domain of NS3 that processes all four downstream sites of the HCV polyprotein (C. Lin et al., J. Virol. 68, 8147-8157 (1994)).

HCV has three structural proteins, the N-terminal nucleocapsid protein (termed “core”) and two envelope glycoproteins, “E1” (also known as E) and “E2” (also known as E2/NS1). See, Houghton et al. (1991) Hepatology 14:381-388, for a discussion of HCV proteins, including E1 and E2. The E1 protein is detected as a 32-35 kDa species and is converted into a single endo H-sensitive band of approximately 18 Kda. By contrast, E2 displays a complex pattern upon immunoprecipitation consistent with the generation of multiple species (Grakoui et al. (1993) J. Virol. 67:1385-1395; Tomei et al. (1993) J. Virol. 67:4017-4026). The HCV envelope glycoproteins E1 and E2 form a stable complex that is co-immunoprecipitable (Grakoui et al. (1993) J. Virol. 67:1385-1395; Lanford et al. (1993) Virology 197:225-235; Ralston et al. (1993) J. Virol. 67:6753-6761).

Antiviral Peptides

In one embodiment, the invention provides an antiviral peptide. An antiviral peptide is a peptide that can prevent or reduce infection of a virus of the family Flaviviridae, herein a peptide inhibitor or a peptide of the invention. Examples of viruses of the Flaviviridae family include, without limitation, the Yellow fever virus, the West Nile virus, the virus that causes Dengue Fever and the Hepatitis C virus.

A Flaviviridae is a spherical, enveloped virus having a linear, single-stranded RNA genome of positive polarity. The family Flaviviridae includes the genera Flavivirus, Hepacivirus and Pestivirus. The invention contemplates treatment of Flaviviridae infections, including infections caused by any virus from any of the genera Flavivirus, Hepacivirus and Pestivirus, as well as viruses of the unassigned genera of Flaviviridae. For example, the present peptides can be used to treat infections caused by the following viruses of the Flavivirus genus: Tick-borne encephalitis, Central European encephalitis, Far Eastern encephalitis, Rio Bravo, Japanese encephalitis, Kunjin, Murray Valley encephalitis, St Louis encephalitis, West Nile encephalitis, Tyulenly, Ntaya, Uganda S, Dengue type 1, Dengue type 2, Dengue type 3, Dengue type 4, Modoc, and Yellow Fever. Moreover, the present peptides can be used to treat infections caused by the following viruses of the Pestivirus genus: Bovine viral diarrhea virus 1, Bovine viral diarrhea virus 2, Hog cholera (classical swine fever virus), and Border disease virus. In addition, the present peptides can be used to treat infections caused by Hepatitis C virus, which is classified in the Hepacivirus genus. Viruses of the unassigned genera of Flaviviridae, whose infections can also be treated with the peptides of the invention include: GB virus-A, GB virus-B and GB virus-C.

To determine the level of antiviral activity a peptide has against one or more members of the Flaviviridae family, and an appropriate dosage for such a peptide, methods known in the art, including, without limitation, those described herein can be used. Viral infection in the presence or absence of a peptide of the invention can be evaluated, for example, by determining intracellular viral RNA levels or the number of viral foci by immunoassays using antibody against viral proteins as described herein. The antiviral activity of a peptide can also be determined using the liposome release assay as exemplified herein. A peptide has antiviral activity if can inhibit or reduce viral infection by any amount, for example, by 2 fold or more than 2 fold. For example, a peptide of the invention can inhibit or reduce HCV infection by 2-5 fold, 5-10 fold, or more than 10 fold. As illustrated hereinbelow, many of the peptides listed in Table 3 can inhibit HCV infection by more than ten-fold, including, for example, peptides with SEQ ID NO:6, 8, 12, 13, 14, 24, 27, 30, 32, 43, 44, 47, 48 and 53. Other peptides listed in Table 3 can inhibit HCV infection by five-fold to ten-fold, including peptides with SEQ ID NO:21, 23, 28 and 37. The remainder of the peptides inhibit HCV infection by at least two-fold and some of the remaining peptides inhibit HCV infection by up to about five-fold. These peptides exhibit such inhibition of viral infection at concentrations of nanomolar and low micromolar levels.

A peptide of the invention is a polymer of α-amino acids linked by amide bonds between the α-amino and α-carboxyl groups. Thus, the term “amino acid,” as used herein, refers to an α-amino acid. The amino acids included in the peptides of the invention can be L-amino acids or D-amino acids. Moreover, the amino acids used in the peptides of the invention can be naturally-occurring and non-naturally occurring amino acids. Thus, a peptide of the present invention can be made from genetically encoded amino acids, naturally occurring non-genetically encoded amino acids, or synthetic amino acids. The amino acid notations used herein for the twenty genetically encoded L-amino acids and some examples of non-encoded amino acids are provided in Table 1.

TABLE 1

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| Â-Alanine | | Bala |
| 2,3-Diaminopropionic | | Dpr |
| acid | | |
| Á-Aminoisobutyric acid | | Aib |
| N-Methylglycine | | MeGly |
| (sarcosine) | | |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydro- | | Tic |
| isoquinoline-3-carboxylic | | |
| acid | | |
| Â-2-thienylalanine | | Thi |
| Methionine sulfoxide | | MSO |
| Homoarginine | | Harg |
| N-acetyl lysine | | AcLys |
| 2,4-Diamino butyric acid | | Dbu |
| Ñ-Aminophenylalanine | | Phe(pNH₂) |
| N-methylvaline | | MeVal |
| Homocysteine | | Hcys |
| Homoserine | | Hser |
| α-Amino hexanoic acid | | Aha |
| α-Amino valeric acid | | Ava |
| 2,3-Diaminobutyric acid | | Dab |

A peptide of the invention will include at least 8 to about 50 amino acid residues, usually about 14 to 40 amino acids, more usually fewer than about 35 or fewer than about 25 amino acids in length. A peptide of the invention will be as small as possible, while still maintaining substantially all of the activity of a larger peptide. Thus, a peptide of the invention may be 8, 9, 10, 11, 12 or 13 amino acids in length. Moreover, the length of the peptide selected by one of skilled in the art may relate to the stability and/or sequence of the peptide. Thus, for example, while peptide 1 (SEQ ID NO:43) exhibits optimal antiviral activity when it has about 18 amino acids, and truncations from the C-terminal end do not eliminate its antiviral activity, until five or so amino acids are deleted. Nonetheless, peptides with sequences different from SEQ ID NO:43 may exhibit optimal activity when they are longer than 18 amino acids or shorter than 13 amino acids. This may be due to sequence differences that stabilize or modify the secondary structure of the peptide. In addition, the peptides can be derivatized with agents that enhance the stability and activity of the peptides. For example, peptides can be modified by attachment of a dansyl moiety or by incorporation of non-naturally occurring amino acids so as to improve the activity and/or conformation stability of the peptides. Use of non-natural amino acids and dansyl moieties can also confer resistance to protease cleavage. It may also be desirable in certain instances to join two or more peptides together in one peptide structure.

The invention is also directed to peptidomimetics of the antiviral peptides of the invention. Peptidomimetics are structurally similar to peptides having peptide bonds, but have one or more peptide linkages optionally replaced by a linkage such as, —CH₂NH—, —CH₂S—, —CH₂—CH₂—, —CH═CH— (cis and trans), —COCH₂—, —CH(OH)CH₂—, and —CH₂SO—, by methods known in the art. Thus, a peptidomimetic is a peptide analog, such as those commonly used in the pharmaceutical industry as non-peptide drugs, that has properties analogous to those of the template peptide. (Fauchere, J., Adv. Drug Res., 15: 29 (1986) and Evans et al., J. Med. Chem., 30:1229 (1987)). Advantages of peptide mimetics over natural peptide embodiments may include more economical production, greater chemical stability, altered specificity and enhanced pharmacological properties such as half-life, absorption, potency and efficacy.

In some embodiments, the amino acid residues of a peptide of the invention can form an amphipathic α-helical structure in solution.

The term “α-helix” refers to a right-handed coiled conformation. In a polypeptide, the α-helical structure results from hydrogen bonding between the backbone N—H group of one amino acid and the backbone C═O group of an amino acid four residues earlier. An α-helix has 3.6 amino acid residues per turn. Certain amino acid residues tend to contribute to the formation of α-helical structures in polypeptides, for example, alanine, cysteine, leucine, methionine, glutamate, glutamine, histidine and lysine.

However, formation of an α-helix also depends upon the solution, pH and temperature in which a peptide resides. Thus, according to the invention, the inventive peptides are α-helical in aqueous solution. The aqueous solution can, for example, have a physiological pH, and/or physiological salts. In general, the amphipathic α-helical structures of the present peptides are detected at moderate temperatures, such as at about 4° C. to about 50° C., or at about room temperature to about body temperature. Thus, for example, the peptides α-helical structure under physiological temperatures and physiological pH values.

An α-helical structure can be detected using methods known in the art including, without limitation, circular dichroism spectroscopy (CD), nuclear magnetic resonance (NMR), crystal structure determination and optical rotary dispersion (ORD).

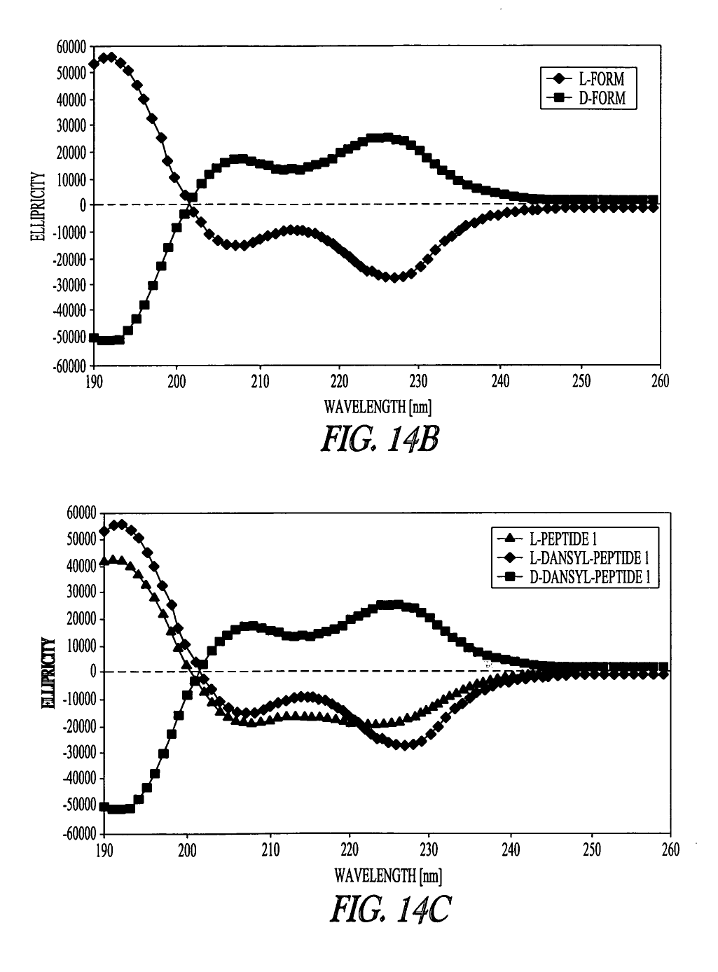

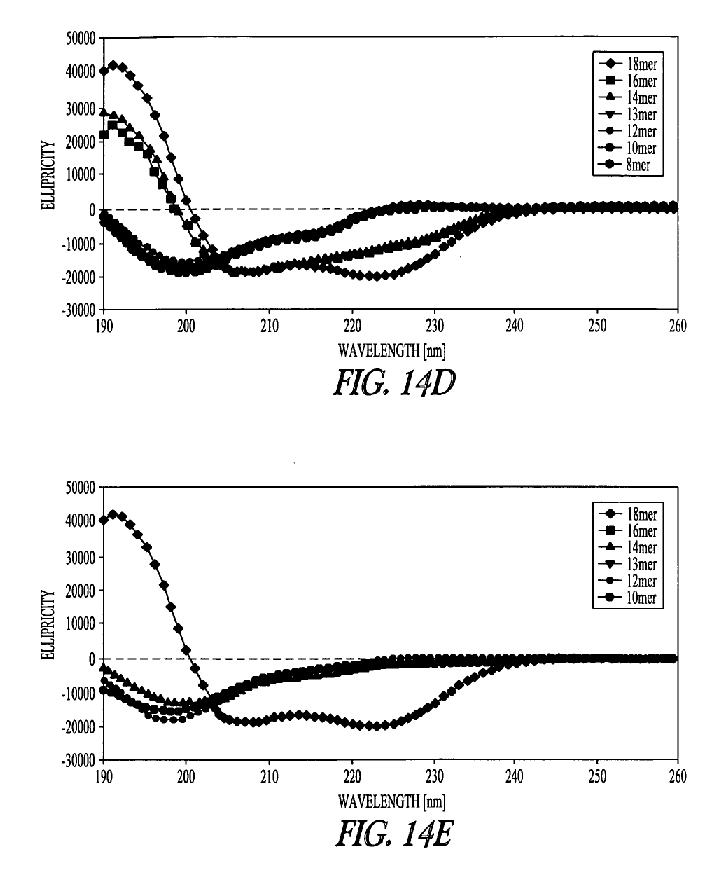

As used herein, the phrase “amphipathic” means that the α-helical peptides have a hydrophilic (or polar) face and a hydrophobic (or non-polar) face, wherein such a “face” refers to a longitudinal surface of the peptide. A helical wheel is apparent when an α-helical peptide is viewed down its longitudinal axis (e.g. as shown in FIG. 14A), one side of the helical wheel that circles this longitudinal axis is composed of hydrophilic (or polar) residues and the other side of the helical wheel is composed of hydrophobic (or nonpolar) residues. Thus, when the peptides of the invention lie on a hydrophilic surface, the hydrophilic face of the peptide will tend to be in contact with the hydrophilic surface. One the other hand, when confronted with a hydrophobic surface, the hydrophobic face of the peptides of the invention will tend to be in contact with the hydrophobic surface.

In an amphipathic α-helical peptide, the hydrophilic and hydrophobic faces of the α-helix can therefore be identified based on the nature of the amino acids present. The hydrophilic face of an α-helix will consist of a larger number of hydrophilic, charged and/or polar amino acids than is present on the hydrophobic face. The hydrophobic face of an amphipathic α-helix consists of hydrophobic and/or non-polar amino acids that facilitate insertion into lipid bilayers. The hydrophobic face may have one or more hydrophilic or polar amino acid as long as a sufficient number of non-polar amino acids are present that enable membrane insertion. In general, a majority of the amino acid residues on the hydrophilic face of the α-helix are charged or otherwise polar amino acids, while a majority of the amino acid residues on the hydrophobic face of the α-helix are non-polar amino acids. Thus in many embodiments, the hydrophilic face of the α-helix consists of charged or otherwise polar amino acids, while the hydrophobic face of the α-helix consists of non-polar amino acid residues. See for example, the helical wheel of the peptide 1 (SEQ ID NO:43), which is shown in FIG. 14A.

Whether any given peptide sequence has a sufficient number of non-polar amino acids to enable membrane insertion can be determined using methods that are well known in the art, including without limitation, methods involving liposomal dye release described in the examples herein. In addition, whether a peptide has an amphipathic α-helical structure can be determined using software available on the internet such as http://cti.itc.virginia.edu/˜cmg/Demo/wheel/wheelApp.html (last visited Aug. 15, 2006) and http://www.bioinfman.ac.uk/˜gibson/ HelixDraw/helixdraw.html (last visited Aug. 15, 2006). A schematic diagram illustrating the amphipathic a-helical structure of the peptide of SEQ ID NO: 43 is shown in FIG. 14A.

Examples of peptides of the invention can be found in Table 3. Other peptides of the invention include those peptides having conservative amino acid substitutions compared to those shown in Table 3. Peptides of the invention also include those having amino acid compositions that resemble the peptides shown in Table 3. These include peptides that have sequences of SEQ ID NO: 96, 97 and 98, which are shown in Table 9. These sequences correspond to the reverse variant of SEQ ID NO: 43 or they constitute a “scrambled” variant of SEQ ID NO: 43. A retro or reverse variant of a peptide such as SEQ ID NO 43 will have an amino acid composition that resembles that of the original peptide (SEQ ID NO: 43), but the amino acid sequence will be the reverse of that of the original peptide. The scrambled variant of a peptide such as SEQ ID NO: 43 will also have an amino acid composition that resembles the original peptide (SEQ ID NO: 43), but the order of the amino acid will be scrambled or mixed up without altering the relative positions of the hydrophobic and hydrophilic residues. Thus, a peptide that is a “hydrophobic scrambled” variant of SEQ ID NO: 43 will have the same amino acid composition as that of SEQ ID NO: 43. However, the order of the hydrophobic amino acid residues will be altered without altering the relative positions of hydrophobic and hydrophilic residues within the sequence such that the amphipathicity of the variant peptide resembles that of the original peptide. Similarly, a “hydrophilic scrambled” variant of SEQ ID NO: 43 will have the same amino acid composition as that of SEQ ID NO: 43, but the order of the hydrophilic amino acid residues will be altered without altering the relative positions of hydrophobic and hydrophilic residues within the sequence such that the amphipathicity of the variant peptide resembles that of the original peptide. In general, the term “scrambling” or “scrambled,” with respect to a hydrophilic (polar) amino acid, is used to indicate that while the positions of each hydrophilic (polar) amino acid are held constant, any other hydrophilic (polar) amino acid can be placed at that position. Similarly, the term “scrambling” or “scrambled,” with respect to a hydrophobic (nonpolar) amino acid, is used to indicate that while the positions of each hydrophobic (nonpolar) amino acid are held constant, any other hydrophobic (nonpolar) amino acid can be placed at that position.

Thus, a peptide of the invention will have an amino acid sequence that is identical to the sequences shown in Table 3, as well as variants of such sequences. Such variants can result from one or more amino acid truncations, conservative substitutions, scrambling of just the hydrophilic amino acids, scrambling of just the hydrophobic residues within a sequence, scrambling of both hydrophilic and hydrophobic amino acids, replacement of naturally occurring amino acids with non-naturally occurring amino acids or other modifications such as dansylation. Such variant peptides are further described in the next section.

Peptides Homologues and Variants

The invention embraces numerous peptide homologues and variants.

A peptide homologue is a peptidyl sequence from an HCV isolate other than the H77 isolate having SEQ ID NO:1. Thus, a peptide of the invention can be a homologue of a peptide with an amino acid sequence of any of SEQ ID NO:4-61. Thus, for example, one peptide homologue of the invention has SEQ ID NO:62, which is a homologue of peptide SEQ ID NO:6.

| | |
|---|---|
| LYGNEGLGWAGWLLSPRG. | (SEQ ID NO:62) |

The sequence of peptide inhibitor SEQ ID NO:62 is found in HCV polyprotein sequences SEQ ID NO:2 and 3.

Another peptide inhibitor homologue of the invention has SEQ ID NO:63 or 64, which are homologues of peptide SEQ ID NO:8.

| | |
|---|---|
| IFLLALLSCITVPVSAAQ; | (SEQ ID NO:63) |
| IFLLALLSCLTIPASAYE. | (SEQ ID NO:64) |

The sequences of these peptide inhibitors are found in HCV polyprotein sequences SEQ ID NO:2 and 3.

Another peptide inhibitor homologue of the invention has SEQ ID NO:65 or 66, which are homologues of peptide SEQ ID NO:12.

| | |
|---|---|
| MSATFCSALYVGDLCGGV | (SEQ ID NO:65) |
| GAAALCSAMYVGDLCGSV | (SEQ ID NO:66) |

The sequences of these peptide inhibitors are found in HCV polyprotein sequences SEQ ID NO:2 and 3.

Another peptide inhibitor homologue of the invention has SEQ ID NO:67 or 68, which are homologues of peptide SEQ ID NO:13.

| | |
|---|---|
| ALYVGDLCGGVMLAAQVF | (SEQ ID NO:67) |
| AMYVGDLCGSVFLVAQLF | (SEQ ID NO:68) |

The sequences of these peptide inhibitors are found in HCV polyprotein sequences SEQ ID NO:2 and 3.

Another peptide inhibitor homologue of the invention has SEQ ID NO:69 or 70, which are homologues of peptide SEQ ID NO:14.

| | |
|---|---|
| IIDIVSGAHWGVMFGLAY | (SEQ ID NO:69) |
| VVDMVAGAHWGVLAGLAY | (SEQ ID NO:70) |

The sequences of these peptide inhibitors are found in HCV polyprotein sequences SEQ ID NO:2 and 3.

Another peptide inhibitor homologue of the invention has SEQ ID NO:71 or 72, which are homologues of peptide SEQ ID NO:24.

| | |
|---|---|
| VDVQYMYGLSPAITKYVV | (SEQ ID NO:71) |
| YLYGIGSAVVSFAIKWEY | (SEQ ID NO:72) |

The sequences of these peptide inhibitors are found in HCV polyprotein sequences SEQ ID NO:2 and 3.

Another peptide inhibitor homologue of the invention has SEQ ID NO:73 or 74, which are homologues of peptide SEQ ID NO:27.

| | |
|---|---|
| WMLILLGQAEAALEKLVV | (SEQ ID NO:73) |
| WMMLLIAQAEAALENLVV | (SEQ ID NO:74) |

The sequences of these peptide inhibitors are found in HCV polyprotein sequences SEQ ID NO:2 and 3.

Another peptide inhibitor homologue of the invention has SEQ ID NO:75 or 76, which are homologues of peptide SEQ ID NO:30.

| | |
|---|---|
| GVVFDITKWLLALLGPAY; | (SEQ ID NO:75) |
| ELIFTITKILLAILGPLM. | (SEQ ID NO:76) |

The sequences of these peptide inhibitors are found in HCV polyprotein sequences SEQ ID NO:2 and 3.

In another embodiment, the peptide inhibitor homologue has SEQ ID NO:77 or 78, which are homologues of peptide SEQ ID NO:32.

| | |
|---|---|
| VSQSFLGTTISGVLWTVY; | (SEQ ID NO:77) |
| ATQSFLATCVNGVCWTVY. | (SEQ ID NO:78) |

The sequences of these peptide inhibitors are found in HCV polyprotein sequences SEQ ID NO:2 and 3.

In another embodiment, the peptide inhibitor homologue has SEQ ID NO:79 or 80, which are homologues of peptide SEQ ID NO:43.

| | |
|---|---|
| SWLRDVWDWVCTILTDFK; | (SEQ ID NO:79) |
| SWLRDVWDWICTVLTDFK. | (SEQ ID NO:80) |

The sequences of these peptide inhibitors are found in HCV polyprotein sequences SEQ ID NO:2 and 3.

In another embodiment, the peptide inhibitor homologue has SEQ ID NO:81 or 82, which are homologues of peptide SEQ ID NO:44.

| | |
|---|---|
| DWVCTILTDFKNWLTSKL; | (SEQ ID NO:81) |
| DWICTVLTDFKTWLQSKL. | (SEQ ID NO:82) |

The sequences of these peptide inhibitors are found in HCV polyprotein sequences SEQ ID NO:2 and 3.

In another embodiment, the peptide inhibitor homologue has SEQ ID NO:83 or 84, which are homologues of peptide SEQ ID NO:47.

| | |
|---|---|
| ASEDVYCCSMSYTWT; | (SEQ ID NO:83) |
| EDDTTVCCSMSYSW. | (SEQ ID NO:84) |

The sequences of these peptide inhibitors are found in HCV polyprotein sequences SEQ ID NO:2 and 3.

In another embodiment, the peptide inhibitor homologue has SEQ ID NO:85 or 86, which are homologues of peptide SEQ ID NO:53.

| | |
|---|---|
| CTMLVCGDDLVVICESAG; | (SEQ ID NO:85) |
| PTMLVCG DDLVVISESQG. | (SEQ ID NO:86) |

The sequences of these peptide inhibitors are found in HCV polyprotein sequences SEQ ID NO:2 and 3.

A peptide variant is any peptide having an amino acid sequence that is not identical to a segment in the polyprotein sequence of a HCV isolate. Thus, a peptide of the invention can have a variant sequence that results from conservative amino acid substitutions. Amino acids that are substitutable for each other generally reside within similar classes or subclasses. As known to one of skill in the art, amino acids can be placed into different classes depending primarily upon the chemical and physical properties of the amino acid side chain. For example, some amino acids are generally considered to be hydrophilic or polar amino acids and others are considered to be hydrophobic or nonpolar amino acids. Polar amino acids include amino acids having acidic, basic or hydrophilic side chains and nonpolar amino acids include amino acids having aromatic or hydrophobic side chains. Nonpolar amino acids may be further subdivided to include, among others, aliphatic amino acids. The definitions of the classes of amino acids as used herein are as follows. “Nonpolar Amino Acid” refers to an amino acid having a side chain that is uncharged at physiological pH, that is not polar and that is generally repelled by aqueous solution. Examples of genetically encoded hydrophobic amino acids include Ala, Ile, Leu, Met, Trp, Tyr and Val. Examples of non-genetically encoded nonpolar amino acids include t-BuA, Cha and Nle.

“Aromatic Amino Acid” refers to a nonpolar amino acid having a side chain containing at least one ring having a conjugated [symbol] -electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, â-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Aliphatic Amino Acid” refers to a nonpolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Polar Amino Acid” refers to a hydrophilic amino acid having a side chain that is charged or uncharged at physiological pH and that has a bond in which the pair of electrons shared in common by two atoms is held more closely by one of the atoms. Polar amino acids are generally hydrophilic, meaning that they have an amino acid having a side chain that is attracted by aqueous solution. Examples of genetically encoded polar amino acids include asparagine, cysteine, glutamine, lysine and serine. Examples of non-genetically encoded polar amino acids include citrulline, homocysteine, N-acetyl lysine and methionine sulfoxide.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include amino acids ornithine, 2,3-diaminopropionic acid, 2,4-diaminobutyric acid and homoarginine.

“Ionizable Amino Acid” refers to an amino acid that can be charged at a physiological pH. Such ionizable amino acids include acidic and basic amino acids, for example, D-aspartic acid, D-glutamic acid, D-histidine, D-arginine, D-lysine, D-hydroxylysine, D-ornithine, L-aspartic acid, L-glutamic acid, L-histidine, L-arginine, L-lysine, L-hydroxylysine or L-ornithine.

As will be appreciated by those having skill in the art, the above classifications are not absolute. Several amino acids exhibit more than one characteristic property, and can therefore be included in more than one category.

For example, tyrosine has both a nonpolar aromatic ring and a polar hydroxyl group. Thus, tyrosine has several characteristics that could be described as nonpolar, aromatic and polar. However, the nonpolar ring is dominant and so tyrosine is generally considered to be hydrophobic. Similarly, in addition to being able to form disulfide linkages, cysteine also has nonpolar character.

Thus, while not strictly classified as a hydrophobic or nonpolar amino acid, in many instances cysteine can be used to confer hydrophobicity or nonpolarity to a peptide.

The classifications of the above-described genetically encoded and non-encoded amino acids are summarized in Table 2, below. It is to be understood that Table 2 is for illustrative purposes only and does not purport to be an exhaustive list of amino acid residues that may comprise the peptides and peptide analogues described herein. Other amino acid residues that are useful for making the peptides described herein can be found, e.g., in Fasman, 1989, CRC Practical Handbook of Biochemistry and Molecular Biology, CRC Press, Inc., and the references cited therein. Another source of amino acid residues is provided by the website of RSP Amino Acids Analogues, Inc. (www.amino-acids.com). Amino acids not specifically mentioned herein can be conveniently classified into the above-described categories on the basis of known behavior and/or their characteristic chemical and/or physical properties as compared with amino acids specifically identified.

TABLE 2

| Classification | Genetically Encoded | Non-Genetically Encoded |
|---|---|---|
| Nonpolar | | |
| Aromatic | F, Y, W | Phg, Nal, Thi, Tic, Phe(4- |
| | | Cl), Phe(2-F), Phe(3-F), |
| | | Phe(4-F), Pyridyl Ala, |
| | | Benzothienyl Ala |
| Aliphatic | A, V, L, I | t-BuA, t-BuG, MeIle, Nle, |
| | | MeVal, Cha, bAla, MeGly, |
| | | Aib |
| Other | M, G, P | |
| Nonpolar | | |
| Polar | | |
| Acidic | D, E | |
| Basic | H, K, R | Dpr, Orn, hArg, Phe(p- |
| | | NH₂), DBU, A₂ BU |
| Neutral Polar | S, T, Y, Q, N, D, E, H, | Cit, AcLys, MSO, hSer, |
| | R, K, C | Orn, Hcys |
| Cysteine-Like | C | Pen, hCys, β-methyl Cys |

In some embodiments, hydrophilic or polar amino acids contemplated by the present invention include, for example, arginine, asparagine, aspartic acid, cysteine, glutamic acid, glutamine, histidine, homocysteine, lysine, hydroxylysine, ornithine, serine, threonine, and structurally related amino acids. In one embodiment the polar amino is an ionizable amino acid such as arginine, aspartic acid, glutamic acid, histidine, hydroxylysine, lysine, or ornithine.

Examples of hydrophobic or nonpolar amino acid residues that can be utilized include, for example, alanine, valine, leucine, methionine, isoleucine, phenylalanine, tryptophan, tyrosine and the like.

In addition, the amino acid sequence of a peptide can be modified so as to result in a peptide variant that includes the substitution of at least one amino acid residue in the peptide for another amino acid residue, including substitutions that utilize the D rather than L form.

One or more of the residues of the peptide can be exchanged for another, to alter, enhance or preserve the biological activity of the peptide. Such a variant can have, for example, at least about 10% of the biological activity of the corresponding non-variant peptide. Conservative amino acid substitutions are often utilized, i.e., substitutions of amino acids with similar chemical and physical properties, as described above.

Hence, for example, conservative amino acids substitutions involve exchanging aspartic acid for glutamic acid; exchanging lysine for arginine or histidine; exchanging one nonpolar amino acid (alanine, isoleucine, leucine, methionine, phenylalanine, tryptophan, tyrosine, valine) for another; and exchanging one polar amino acid (aspartic acid, asparagine, glutamic acid, glutamine, glycine, serine, threonine, etc.) for another. When substitutions are introduced, the variants can be tested to confirm or determine their levels of biological activity.

For example, in some embodiments, the peptides of the invention can have a sequence that includes any one of formulae I-V:

| | |
|---|---|
| I | |
| Xaa₁-Xaa₂-Xaa₃-Xaa₄-Xaa₅-Xaa₆- | (SEQ ID NO: 112) |
| Xaa₇-Xaa₈-Xaa₉-Xaa₁₀-Xaa₁₁-Xaa₁₂- | |
| Xaa₁₃-Xaa₁₄ | |
| II | |
| Xaa₁-Xaa₂-Xaa₃-Xaa₄-Xaa₅-Xaa₆- | (SEQ ID NO: 113) |
| Xaa₇-Xaa₈-Xaa₉-Xaa₁₀-Xaa₁₁-Xaa₁₂- | |
| Xaa₁₃-Xaa₁₄-Xaa₁₅ | |
| III | |
| Xaa₁-Xaa₂-Xaa₃-Xaa₄-Xaa₅-Xaa₆- | (SEQ ID NO: 114) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆ | |
| IV | |
| Xaa₁-Xaa₂-Xaa₃-Xaa₄-Xaa₅-Xaa₆- | (SEQ ID NO: 115) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇ | |
| V | |
| Xaa₁-Xaa₂-Xaa₃-Xaa₄-Xaa₅-Xaa₆- | (SEQ ID NO: 116) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇- | |
| Xaa₁₈ | |

wherein:

Xaa₁, Xaa₄, Xaa₅, Xaa₈, Xaa₁₁, Xaa₁₂, Xaa₁₅, Xaa₁₆ and Xaa₁₈ are polar amino acids; and

Xaa₂, Xaa₃, Xaa₆, Xaa₇, Xaa₉, Xaa₁₀, Xaa₁₃, Xaa₁₄, and Xaa₁₇ are nonpolar amino acids.

In other embodiments, the present peptides can have additional peptidyl sequences at either the N-terminus or the C-terminus. Thus, for example, the invention provides a fusion peptide formed by attaching a 14 amino acid peptide (the N-terminyl peptide) to the N-terminus of a peptide of any of formulae I to V. The 14 amino acid N-terminyl peptide has the structure: Rx-Ry-Ry-Rx-Ry-Ry-Rx-Rx-Ry-Ry-Rx-Rx-Ry-Rx (SEQ ID NO: 117), wherein each Rx is separately a polar amino acid, and each Ry is separately a nonpolar amino acid.

The invention also provides a fusion peptide formed by attaching a 12 amino acid peptide (the C-terminyl peptide) to the C-terminus of a peptide of formula V. The resulting fusion peptide has the structure of formulae VI:

| | |
|---|---|
| VI | |
| Xaa₁-Xaa₂-Xaa₃-Xaa₄-Xaa₅-Xaa₆- | (SEQ ID NO: 118) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇- | |
| Xaa₁₈-Xaa₁₉-Xaa₂₀-Xaa₂₁-Xaa₂₂- | |
| Xaa₂₃-Xaa₂₄-Xaa₂₅-Xaa₂₆-Xaa₂₇- | |
| Xaa₂₈-Xaa₂₉-Xaa₃₀, | |

wherein:

Xaa₁, Xaa₄, Xaa₅, Xaa₈, Xaa₁₁, Xaa₁₂, Xaa₁₅, Xaa₁₆, Xaa₁₈, Xaa₁₉, Xaa₂₂, Xaa₂₃, Xaa₂₆, Xaa₂₉, and Xaa₃₀ are separately each a polar amino acid; and

Xaa₂, Xaa₃, Xaa₆, Xaa₇, Xaa₉, Xaa₁₀, Xaa₁₃, Xaa₁₄, Xaa₁₇, Xaa₂₀, Xaa₂₁, Xaa₂₄, Xaa₂₅, Xaa₂₇, and Xaa₂₈ are separately each a nonpolar amino acid.

The invention also provides a fusion peptide having a sequence that corresponds to the 14 amino acid N-terminyl peptide of SEQ ID NO: 117 attached by a peptide bond to the N-terminus of a peptide of formula VI.

In another embodiment, a peptide of the invention is a peptide comprising at least 14 contiguous amino acids of any of the above described peptides.

A peptide variant can also result from “scrambling” of the hydrophilic and/or hydrophobic residues within a sequence as long as the amphipathic α-helical secondary structure of the peptide in solution is maintained.

Methods of Making a Peptide of the Invention In the context of the present invention, an “isolated” peptide is a peptide that exists apart from its native environment and is therefore not a product of nature. An isolated peptide may exist in a purified form or may exist in a non-native environment such as, for example, in a cell or in a composition with a solvent that may contain other active or inactive ingredients. In one embodiment, an “isolated” peptide free of at least some of sequences that naturally flank the peptide (i.e., sequences located at the N-terminal and C-terminal ends of the peptide) in the protein from which the peptide was originally derived. A “purified” peptide is substantially free of other cellular material, or culture medium when produced by recombinant techniques, or substantially free of chemical precursors or other chemicals when chemically synthesized. Thus, a purified peptide preparation is at least 50%, at least 60%, at least 70%, at least 80% or at least 90% by weight peptide. Purity can be determined using methods known in the art, including, without limitation, methods utilizing chromatography or polyacrylamide gel electrophoreseis.

The present peptides or variants thereof, can be synthesized in vitro, e.g., by the solid phase peptide synthetic method or by enzyme catalyzed peptide synthesis or with the aid of recombinant DNA technology. Solid phase peptide synthetic method is an established and widely used method, which is described in references such as the following: Stewart et al., Solid Phase Peptide Synthesis, W. H. Freeman Co., San Francisco (1969); Merrifield, J. Am. Chem. Soc. 85 2149 (1963); Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS—PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

Peptides of the invention can be cyclic peptides so long as they retain anti-viral activity. Such cyclic peptides are generated from linear peptides typically by covalently joining the amino terminus to the terminal carboxylate. To insure that only the termini are joined amino and carboxylate side chains can be protected with commercially available protecting groups. In some embodiments, one of skill in the art may choose to cyclize peptide side chains to one of the amino or carboxylate termini, or to another amino acid side chain. In this case, protecting groups can again be used to guide the cyclization reaction as desired.

Cyclization of peptides can be performed using available procedures. For example, cyclization can be performed in dimethylformamide at a peptide concentration of 1-5 mM using a mixture of benzotriazole-1-yl-oxy-tris-pyrrolidino-phosphonium hexafluorophosphate (PyBOP, Novabiochem) (5 eq. with respect to crude peptide) and N,N-diisopropylethylamine (DIEA, Fisher) (40 eq.). The amount of DIEA is adjusted to achieve an apparent pH 9-10. The reaction can be followed by any convenient means, for example, by MALDI-MS and/or HPLC.

N-acyl derivatives of an amino group of the peptide or peptide variants may be prepared by utilizing an N-acyl protected amino acid for the final condensation, or by acylating a protected or unprotected peptide. O-acyl derivatives may be prepared, for example, by acylation of a free hydroxy peptide or peptide resin. Either acylation may be carried out using standard acylating reagents such as acyl halides, anhydrides, acyl imidazoles, and the like. Both N-acylation and O-acylation may be carried out together, if desired.

Salts of carboxyl groups of a peptide or peptide variant of the invention may be prepared in the usual manner by contacting the peptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

Acid addition salts of the peptide or variant peptide, or of amino residues of the peptide or variant peptide, may be prepared by contacting the peptide or amine with one or more equivalents of the desired inorganic or organic acid, such as, for example, hydrochloric acid. Esters of carboxyl groups of the peptides may also be prepared by any of the usual methods known in the art.

Methods of Use

Peptide of the invention can be employed to prevent, treat or otherwise ameliorate infection by a virus of the Flaviviridae family, which includes, without limitation, viruses in the genera Flavivirus, Pestivirus, and Hepacivirus, as described above. Members of the Flavivirus genus include viruses that cause Tick-borne encephalitis, Central European encephalitis, Far Eastern encephalitis, Rio Bravo, Japanese encephalitis, Kunjin, Murray Valley encephalitis, St Louis encephalitis, West Nile encephalitis, Tyulenly, Ntaya, Uganda S, Dengue type 1, Dengue type 2, Dengue type 3, Dengue type 4, Modoc, and Yellow Fever. Members of the Pestivirus genus include Bovine viral diarrhea virus 1, Bovine viral diarrhea virus 2, Hog cholera (classical swine fever virus), and Border disease virus. The Hepacivirus genus include Hepatitis C virus. Additional members of the Flaviviridae family include the unassigned GB virus-A, GB virus-B, and GB virus-C. Members of the Flaviviridae family of viruses are known to cause a variety of diseases including, for example, Dengue fever, Hepatitis C infection, Japanese encephalitis, Kyasanur Forest disease, Murray Valley encephalitis, St. Louis encephalitis, Tick-borne encephalitis, West Nile encephalitis and Yellow fever.

A peptide of the invention can be used to prevent, treat or otherwise ameliorate infection by a member of the Flaviviridae family of viruses and its associated disease conditions. Thus, examples of various applications of the invention include, without limitation, use as a therapeutic for patients with Dengue fever, Dengue hemorrhagic fever, Dengue shock syndrome, Japanese aencephalitis, Kyasanur forest disease, Murray Valley encephalitis, St. Louis Encephalitis, Tick-borne meningoencephalitis, Chronic hepatitis C infection, to prevent graft infection during liver transplantation, to prevent sexual transmission, to increase the safety of blood and blood product used in transfusions, and to increased safety of clinical laboratory samples.

In one embodiment, the invention provides a method for preventing or otherwise ameliorating viral infection of a mammalian cell, such as a human cell, or a method for preventing, treating or otherwise ameliorating acute or chronic infection, by a virus of the Flaviviridae family, of a mammal such as a human.

As used herein “preventing” is intended to include the administration of a peptide of the invention to a mammal such as a human who could be or has been exposed to a member of the Flaviviridae family. The mammal who could be exposed to a virus of the Flaviviridae family includes, without limitation, someone present in an area where these viruses are prevalent or common, e.g. the tropics, Southeast Asia and the Far East, South Asia, Australia and Papua New guinea, the United States, Russia, Africa, as well as Central and South American countries. The mammal who could be exposed to a virus of the Flaviviridae family also includes someone who has been bitten by a deer or forest tick or a mosquito; a recipient of donated body tissue or fluids, for example, a recipient of blood or one or more of its components such as plasma, platelets, or stem cells; and medical, clinical or dental personnel who handle body tissues and fluids. A mammal who has been exposed to a virus of the Flaviviridae family include, without limitation, someone who has had contact with the body tissue or fluid, e.g. blood, of an infected person or otherwise have come in contact with HCV or any other virus of the Flaviviridae family.

Treatment of, or treating a Flaviviridae viral infection is intended to include a reduction of the viral load or the alleviation of or diminishment of at least one symptom typically associated with the infection. The treatment also includes alleviation or diminishment of more than one symptom. Ideally, the treatment cures, e.g., substantially inhibits viral infection and/or eliminates the symptoms associated with the infection.

Symptoms or manifestations of viral exposure or infection are specific for the particular infection, and these are known in the art. Dengue fever and dengue hemorrhagic fever, for example, is caused by one of four Flavivirus serotypes. Symptoms of these conditions include sudden onset of fever, severe headache, joint and muscular pains and rashes, as well as high fever, thrombocytopenia and haemoconcentration. Clinical indications of also include high fever, petechial rash with thrombocytopenia and leucopenia, and haemorrhagic tendency. Symptoms of Japanese aencephalitis include fever, headache, neck rigidity, cachexia, hemiparesis, convulsions and heightened body temperature. Japanese encephalitis can be diagnosed by detection of antibodies in serum and cerebrospinal fluid. Symptoms of Kyasanur forest disease include high fever, headache, haemorrhages from nasal cavity and throat, and vomiting. Symptoms of St. Louis encephalitis include fever, headache, neck stiffness, stupor, disorientation, coma, tremors, occasional convulsions and spastic paralysis. Symptoms of Murray Valley encephalitis include fever, seizures, nausea and diarrhea in children, and headaches, lethargy and confusion in adults. Symptoms of West Nile virus infection include flu-like symptoms, malaise, fever, anorexia, nausea, vomiting, eye pain, headache, myalgia, rash and lymphadenopathy, as well as encephalitis (inflammation of the brain) and meningitis (inflammation of the lining of the brain and spinal cord), meningismus, temporary blindness, seizures and coma. West Nile infection can be diagnosed using ELISA to detect antibodies in the blood or cerebrospinal fluids. Symptoms of Yellow fever include fever, muscle aches, headache, backache, a red tongue, flushed face, red eyes, hemorrhage from the gastrointestinal tract, bloody vomit, jaundice, liver failure, kidney insufficiency with proteinuria, hypotension, dehydration, delirium, seizure and coma. Symptoms of hepatitis C infection include, without limitation, inflammation of the liver, decreased appetite, fatigue, abdominal pain, jaundice, flu-like symptoms, itching, muscle pain, joint pain, intermittent low-grade fevers, sleep disturbances, nausea, dyspepsia, cognitive changes, depression headaches and mood changes. HCV infection could also be diagnosed by detecting antibodies to the virus, detecting liver inflammation by biopsy, liver cirrhosis, portal hypertension, thyroiditis, cryoglobulinemia and glomerulonephritis. In addition HCV infection could be diagnosed. In addition, diagnosis of exposure or infection or identification of one who is at risk of exposure to HCV could be based on medical history, abnormal liver enzymes or liver function tests during routine blood testing. Generally, infection by a member of the Flaviviridae family can be diagnosed using ELISA for detecting viral antigens or anti-viral antibodies, immunofluorescence for detecting viral antigens, polymerase chain reaction (PCR) for detecting viral nucleic acids and the like.

Methods of preventing, treating or otherwise ameliorating acute or chronic viral infection include contacting the cell with an effective amount of a peptide of the invention or administering to a mammal such as a human a therapeutically effective amount of a peptide of the present invention.

A peptide of the invention can be administered in a variety of ways. Routes of administration include, without limitation, oral, parenteral (including subcutaneous, intravenous, intramuscular and intraperitoneal), rectal, vaginal, dermal, transdermal (topical), transmucosal, intrathoracic, intrapulmonary and intranasal (respiratory) routes. The means of administration may be by injection, using a pump or any other appropriate mechanism.

A peptide of the invention may be administered in a single dose, in multiple doses, in a continuous or intermittent manner, depending, for example, upon the recipient's physiological condition, whether the purpose of the administration is therapeutic or prophylactic, and other factors known to skilled practitioners. The administration of the peptides of the invention may be essentially continuous over a pre-selected period of time or may be in a series of spaced doses. Both local and systemic administration is contemplated.

The dosage to be administered to a mammal may be any amount appropriate to reduce or prevent viral infection or to treat at least one symptom associated with the viral infection. Some factors that determine appropriate dosages are well known to those of ordinary skill in the art and may be addressed with routine experimentation. For example, determination of the physicochemical, toxicological and pharmacokinetic properties may be made using standard chemical and biological assays and through the use of mathematical modeling techniques known in the chemical, pharmacological and toxicological arts. The therapeutic utility and dosing regimen may be extrapolated from the results of such techniques and through the use of appropriate pharmacokinetic and/or pharmacodynamic models. Other factors will depend on individual patient parameters including age, physical condition, size, weight, the condition being treated, the severity of the condition, and any concurrent treatment. The dosage will also depend on the peptide(s) chosen and whether prevention or treatment is to be achieved, and if the peptide is chemically modified. Such factors can be readily determined by the clinician employing viral infection models such as the HCV cell culture/JFH-1 infection model described herein, or other animal models or test systems that are available in the art.

The precise amount to be administered to a patient will be the responsibility of the attendant physician. However, to achieve the desired effect(s), a peptide of the invention, a variant thereof or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results.

The absolute weight of a given peptide included in a unit dose can vary widely. For example, about 0.01 to about 2 g, or about 0.1 to about 500 mg, of at least one peptide of the invention, or a plurality of peptides specific for a particular cell type can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the peptides of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

A peptide of the invention may be used alone or in combination with a second medicament. The second medicament can be a known antiviral agent such as, for example, an interferon-based therapeutic or another type of antiviral medicament such as ribavirin. The second medicament can be an anticancer, antibacterial, or antiviral agent. The antiviral agent may act at any step in the life cycle of the virus from initial attachment and entry to egress. Thus, the added antiviral agent may interfere with attachment, fusion, entry, trafficking, translation, viral polyprotein processing, viral genome replication, viral particle assembly, egress or budding. Stated another way, the antiviral agent may be an attachment inhibitor, entry inhibitor, a fusion inhibitor, a trafficking inhibitor, a replication inhibitor, a translation inhibitor, a protein processing inhibitor, an egress inhibitor, in essence an inhibitor of any viral function. The effective amount of the second medicament will follow the recommendations of the second medicament manufacturer, the judgment of the attending physician and will be guided by the protocols and administrative factors for amounts and dosing as indicated in the PHYSICIAN'S DESK REFERENCE.

The effectiveness of the method of treatment can be assessed by monitoring the patient for signs or symptoms of the viral infection as discussed above, as well as determining the presence and/or amount of virus present in the blood, e.g. the viral load, using methods known in the art including, without limitation, polymerase chain reaction and transcription mediated amplification.

Pharmaceutical Compositions

In one embodiment, the invention provides a pharmaceutical composition comprising a peptide of the invention. To prepare such a pharmaceutical composition, a peptide of the invention is synthesized or otherwise obtained, purified as necessary or desired and then lyophilized and stabilized. The peptide can then be adjusted to the appropriate concentration and then combined with other agent(s) or pharmaceutically acceptable carrier(s). By “pharmaceutically acceptable” it is meant a carrier, diluent, excipient, and/or salt that is compatible with the other ingredients of the formulation, and not deleterious to the recipient thereof.

Pharmaceutical formulations containing a therapeutic peptide of the invention can be prepared by procedures known in the art using well-known and readily available ingredients. For example, the peptide can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone.

Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For oral administration, a peptide may be present as a powder, a granular formulation, a solution, a suspension, an emulsion or in a natural or synthetic polymer or resin for ingestion of the active ingredients from a chewing gum. The active peptide may also be presented as a bolus, electuary or paste. The formulations may, where appropriate, be conveniently presented in discrete unit dosage forms and may be prepared by any of the methods well known to the pharmaceutical arts including the step of mixing the therapeutic agent with liquid carriers, solid matrices, semi-solid carriers, finely divided solid carriers or combinations thereof, and then, if necessary, introducing or shaping the product into the desired delivery system. The total active ingredients in such formulations comprise from 0.1 to 99.9% by weight of the formulation.

Tablets or caplets containing the peptides of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one peptide of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more peptides of the invention are designed to resist disintegration in the stomach and dissolve in the more neutral to alkaline environment of the duodenum.

Orally administered therapeutic peptide of the invention can also be formulated for sustained release. In this case, a peptide of the invention can be coated, micro-encapsulated (see WO 94/07529, and U.S. Pat. No. 4,962,091), or otherwise placed within a sustained delivery device. A sustained-release formulation can be designed to release the active peptide, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

A therapeutic peptide of the invention can also be formulated as elixirs or solutions for convenient oral administration or as solutions appropriate for parenteral administration, for instance by intramuscular, subcutaneous, intraperitoneal or intravenous routes. A pharmaceutical formulation of a therapeutic peptide of the invention can also take the form of an aqueous or anhydrous solution or dispersion, or alternatively the form of an emulsion or suspension or salve.

Thus, a therapeutic peptide may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The active peptides and other ingredients may form suspensions, solutions, or emulsions in oily or aqueous vehicles, and may contain formulatory agents such as suspending, stabilizing and/or dispersing agents. Alternatively, the active peptides and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

In some embodiments the peptides are formulated as a microbicide, which is administered topically or to mucosal surfaces such as the vagina, the rectum, eyes, nose and the mouth. For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Thus, in one embodiment, a peptide of the invention can be formulated as a vaginal cream or a microbicide to be applied topically. Other conventional forms for this purpose include wound dressings, coated bandages or other polymer coverings, ointments, creams, lotions, pastes, jellies, sprays, and aerosols. Thus, the therapeutic peptides of the invention can be delivered via patches or bandages for dermal administration. Alternatively, the peptide can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active peptides can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic peptides in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic peptide may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The peptides of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of the viral infection. Any statistically significant attenuation of one or more symptoms of the infection that has been treated pursuant to the method of the present invention is considered to be a treatment of such infection within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

A therapeutic peptide of the present invention can also be administered in an aqueous solution when administered in an aerosol or inhaled form. Thus, other aerosol pharmaceutical formulations may comprise, for example, a physiologically acceptable buffered saline solution containing between about 0.1 mg/mL and about 100 mg/mL of one or more of the peptides of the present invention specific for the indication or disease to be treated. Dry aerosol in the form of finely divided solid peptide or nucleic acid particles that are not dissolved or suspended in a liquid are also useful in the practice of the present invention. Peptides of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic peptides of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

A therapeutic peptide of the invention may also be used in combination with one or more known therapeutic agents, for example, a pain reliever; an antiviral agent such as an anti-HBV, anti-HCV (HCV inhibitor, HCV protease inhibitor) or an anti-herpetic agent; an antibacterial agent; an anti-cancer agent; an anti-inflammatory agent; an antihistamine; a bronchodilator and appropriate combinations thereof, whether for the conditions described or some other condition.

Miscellaneous Compositions and Articles of Manufacture

In one embodiment, the invention provides an article of manufacture that includes a pharmaceutical composition containing a peptide of the invention for controlling microbial infections. Such articles may be a useful device such as a vaginal ring, a condom, a bandage or a similar device. The device holds a therapeutically effective amount of a pharmaceutical composition for controlling viral infections. The device may be packaged in a kit along with instructions for using the pharmaceutical composition for control of the infection. The pharmaceutical composition includes at least one peptide of the present invention, in a therapeutically effective amount such that viral infection is controlled.

An article of manufacture may also be a vessel or filtration unit that can be used for collection, processing or storage of a biological sample containing a peptide of the invention. A vessel may be evacuated. Vessels include, without limitation, a capillary tube, a vacutainer, a collection bag for blood or other body fluids, a cannula, a catheter. The filtration unit can be part of another device, for example, a catheter for collection of biological fluids. Moreover, the peptides of the invention can also be adsorbed onto or covalently attached to the article of manufacture, for example, a vessel or filtration unit. Thus, when material in the article of manufacture is decanted therefrom or passed through the article of manufacture, the material will not retain substantial amounts of the peptide. However, adsorption or covalent attachment of the peptide to the article of manufacture kills viruses or prevents their transmission, thereby helping to control viral infection. Thus, for example, the peptides of the invention can be in filtration units integrated into biological collection catheters and vials, or added to collection vessels to remove or inactivate viral particles that may be present in the biological samples collected, thereby preventing transmission of the disease.

The invention also provides a composition comprising a peptide of the invention and one or more clinically useful agents such as a biological stabilizer. Biological stabilizer includes, without limitation, an anticoagulant, a preservative and a protease inhibitor. Anticoagulants include, without limitation, oxalate, ethylene diamine tetraacetic acid, citrate and heparin. Preservatives include, without limitation, boric acid, sodium formate and sodium borate. Protease inhibitors include inhibitors of dipeptidyl peptidase IV. Compositions comprising a peptide of the invention and a biological stabilizer may be included in a collection vessel such as a capillary tube, a vacutainer, a collection bag for blood or other body fluids, a cannula, a catheter or any other container or vessel used for the collection, processing or storage of a biological samples.

The invention also provides a composition comprising a peptide of the invention and a biological sample such as blood, semen or other body fluids that is to be analyzed in a laboratory or introduced into a recipient mammal. For example, a peptide of the invention can be mixed with blood prior to laboratory processing and/or transfusions.

In another embodiment, the peptides of the invention can be included in physiological media used to store and transport biological tissues, including transplantation tissues. Thus, for example, liver, heart, kidney and other tissues can be bathed in media containing the present peptides to inhibit viral transmission to transplant recipients.

The invention is further illustrated by the following non-limiting Examples.

### EXAMPLES

### Example 1

### Materials and Methods

HCV constructs and transcription. The HCV consensus clone used was derived from a Japanese patient with fulminant hepatitis, and has been designated JFH-1 (Kato et al. (2001) J. Med. Virol. 64, 334-339). This HCV cDNA was cloned behind a T7 promoter to create the plasmid pJFH-1, as well as a replication-defective NS5B negative control construct pJFH-1/GND (Kato et al. (2003) Gastroenterology 125, 1808-1817). To generate genomic JFH-1 and JFH-1/GND RNA, the pJFH-1 and pJFH-1/GND plasmids were linearized at the 3′ end of the HCV cDNA by XbaI digestion. The linearized DNA was then purified and used as a template for in vitro transcription (MEGAscript; Ambion, Austin, Tex.). To generate JFH-1 strand-specific RNA probes, the inventors cloned a 1 kb fragment of the JFH-1 NS5B coding region into the pBSKII+ vector to allow for T7 and SP6-driven transcription of JFH-1 negative and JFH-1 positive strand probes, respectively.

Cell culture. The hepatic Huh-7 and Huh-7.5.1 cells, and the non-hepatic HEK293 and HeLa cells were maintained in D-MEM (Invitrogen, Carlsbad, Calif.) supplemented with 10% fetal calf serum (Invitrogen), 10 mM Hepes, 100 units/mL penicillin, 100 mg/mL streptomycin and 2 mM L-glutamine (Invitrogen, Carlsbad, Calif.) at 5% CO₂. The non-hepatic HEK293 cells used in these studies are described in Graham et al. (1977) J. Gen. Virol. 36, 59-74. The HeLa cells employed are described in Gey et al. (1952) Cancer Res. 12, 264-265. The human promyeloblastic HL-60 cells and the monoblastoid U-937 cells were purchased from the American Type Culture Collestion (ATCC) and cultured as recommended. The human hepatocarcinoma cell line HepG2 was obtained from the ATCC and is described in Knowles et al. (1980) Science 209, 497-499). Ebstein-Barr virus-transformed B cells were maintained in RPMI medium with the same supplements described above (Invitrogen).

The cells designated Huh-7.5.1 were derived from the Huh-7.5 GFP-HCV replicon cell line I/5A-GFP-6 (Moradpour (2004) J. Virol. 78, 7400-7409). To cure the HCV-GFP replicon from the I/5A-GFP-6 cells to create the HCV-negative Huh-7.5.1 cell line, the I/5A-GFP-6 replicon cells were cultured for three weeks in the presence of 100 IU/ml human interferon gamma (IFNγ) to eradicate the I/5A-GFP-6 replicon. Clearance of the HCV replicon bearing the neomycin resistance gene was confirmed by G418 sensitivity and HCV-specific reverse transcription quantitative polymerase chain reaction (RT-QPCR) analysis.

HCV RNA transfection. Two different methods were used to transfect in vitro transcribed JFH-1 RNA into Huh-7 and Huh-7.5.1 cells. One method was a modification of the electroporation protocol described in Krieger et al. (2001) J. Virol. 75, 4614-4624. Briefly, trypsinized cells were washed twice with serum-free Opti-MEM (Invitrogen) and then resuspended in the same media at a cell density of 1×10⁷ cells per ml. Ten micrograms of JFH-1 RNA was mixed with 0.4 ml of the cells in a 4-mm cuvette and a Bio-Rad Gene Pulser system (BioRad, Hercules, Calif.) was used to deliver a single pulse at 0.27 kV, 100 ohms, and 960 μF and the cells were plated in a T162 Costar flask (Corning). The second method involved liposome mediated transfection, which was performed with Lipofectamin 2000 (Invitrogen) at an RNA:lipofectamin ratio of 1:2 using 5 μg of JFH-1 RNA in a suspensions of 104 cells in the presence of 20% FCS. Cells were then plated in complete DMEM with 20% FCS for overnight incubation. In both cases, transfected cells were transferred to complete DMEM and cultured for the indicated period of time. Cells were passaged every 3 to 5 days. The presence of HCV in these cells and corresponding supernatants was determined by quantifying the number of HCV RNA copies per μg of total cellular RNA and by determining the HCV infectivity titer of the supernatants at selected time points.

RNA analysis. Total cellular RNA was isolated by the guanidine thiocyanate (GTC) method using standard protocols. Chomczynski et al. (1987) Anal. Biochem. 162, 156-159. RNAse-resistant RNA from the cell supernatant was isolated by a modified GTC extraction protocol. Five micrograms of RNA was subjected to Northern blot analysis as previously described by Guidotti (1995), except that HCV RNA was detected with ³²P—UTP labeled strand-specific probes (Maxiscript; Ambion). Alternatively, one microgram of RNA was DNAse treated (DNA-free reagent; Ambion) and subjected to quantitative RT-PCR. Quantitative RT-PCR analysis was performed as described in Kapadia et al. (2005) Proc. Natl. Acad. Sci. U.S.A. 102, 2561-2566; Kapadia et al. (2003) Proc. Natl. Acad. Sci. U.S.A. 100, 2014-2018. DNAse-treated RNA was used for cDNA synthesis using the TaqMan reverse transcription reagents according to the manufacturer's instructions (Applied Biosystems), followed by real-time quantitative PCR using a BioRad iCyler. HCV and GAPDH transcript levels were determined relative to a standard curve comprised of serial dilutions of plasmid containing the HCV JFH-1 cDNA or human GAPDH gene.

The PCR primer sequences employed to detect human GAPDH (Genbank accession No. NMX002046) were:

| | |
|---|---|
| 5′-GAAGGTGAAGGTCGGAGTC-3′ | (sense, SEQ ID NO:87) |
| and | |
| 5′-GAAGATGGTGATGGGATTTC-3′. | (antisense, |
| | SEQ ID NO:88) |

The PCR primers used to detect JFH-1 were:

| | |
|---|---|
| 5′-TCTGCGGAACCGGTGAGTA-3′ | (sense, SEQ ID NO:89) |
| and | |
| 5′-TCAGGCAGTACCACAAGGC-3′. | (antisense, |
| | SEQ ID NO:90) |

Indirect Immunofluorescence. Intracellular staining was performed as described in Kapadia et al. (2003) Proc. Natl. Acad. Sci. U.S.A. 100, 2014-2018. Cells were fixed for 10 minutes at room temperature (rt) in 4% paraformaldehyde (pH 7.2) and permeabilized for 1 hour at room temperature in blocking buffer containing 0.3% Triton X-100, 3% bovine serum albumin (BSA) and 10% FCS in PBS (pH 7.2). Polyclonal anti-NS5A rabbit antibody MS5 was used at a dilution of 1:1000 in a buffer containing 0.3% Triton X-100, 3% BSA. Cells were then incubated with a 1:1000 dilution of Alexa555-conjugated goat anti-rabbit IgG (Molecular Probes, Eugene, Oreg.) for 1 hour at room temperature. Cell nuclei were visualized using by Hoechst staining.

Titration of infectious HCV supernatants. Infectious viral titer of transfected an/or infected cell supernatants was determined by end point limit dilution analysis. Briefly, cell supernatants were serially diluted 10-fold in complete DMEM and used to infect 104 naïve Huh-7.5.1 cells per well in 96-well plates (Corning). The inoculum was incubated with cells for 1 hour at 37° C. and then supplemented with fresh complete DMEM. The level of HCV infection was determined 3 days post-infection by immunofluorescence staining for HCV NS5A or glycoprotein E2 (red). Cell nuclei were stained by Hoechst dye (blue). The viral titer was expressed as focus forming units per mL of supernatant (ffu/mL), determined by the average number of NS5A-positive foci detected at the highest dilutions.

Amplification of HCV viral stocks. To generate viral stocks, infectious supernatants were diluted in complete DMEM and used to inoculate naïve 10-15% confluent Huh-7.5.1 cells at an MOI of 0.01 in a T75 flask (Corning). Infected cells were trypsinized and re-plated prior to confluence at day 4-5 post-infection (p.i.). Supernatant from infected cells was then harvested 8-9 days post-infection and aliquots were stored at −80° C. The titer of viral stock was determined as described above.

Concentration and purification of HCV. Sucrose density gradient ultracentrifugation analysis was performed as described in Heller et al. (2005) Proc. Natl. Acad. Sci. U.S.A. 102, 2579-2583. Pooled supernatants from two mock or two HCV-infected T162 cm₂ flasks were centrifuged at 4,000 rpm for 5 minutes to remove cellular debris, and then pelleted through a 20% sucrose cushion at 28,000 rpm for 4 h using a SW28 rotor in an L8-80M ultracentrifuge (Beckman Instruments, Palo Alto, Calif.). The pellet was resuspended in 1 ml TNE buffer (50 mM Tris.HCl, pH 8, 100 mM NaCl, 1 mM EDTA) containing protease inhibitors (Roche Applied Science, Indianapolis, Ind.), loaded onto a 20-60% sucrose gradient (12.5-mL total volume), and centrifuged at 120,000×g for 16 hours at 4° C. in a SW41Ti rotor (Beckman Instruments). Fractions of 1.3 mL were collected from the top of the gradient. The fractions were analyzed by quantitative RT-PCR to detect HCV RNA. To determine the infectivity titer of each fraction, aliquots of each fraction were diluted 1:10, 1:100, 1:1000 and 1:10000 in DMEM media and titrated on Huh7.5.1 cells as described above. For all analyses, mock infected Huh-7.5.1 cell supernatants were analyzed in parallel.

Western Blot analysis. Detection of intracellular HCV proteins by Western blot analysis was performed as described in Kapadia, S. B., Brideau-Andersen, A. & Chisari, F. V. (2003) Proc. Natl. Acad. Sci. U.S.A. Antibody to HCV core (C7-50) was obtained from Affinity Bioreagents (Golden, Colo.). Anti-NS3 rabbit antibody (MS15) was a gift from Dr. Michael Houghton (Chiron Corporation, Emeryville, Calif.).

Blocking infection with CD81 and E2 specific antibodies specific. Recombinant human monoclonal anti-E2 antibody was derived from a cDNA expression library (prepared from mononuclear cells of a HCV patient) that was screened against recombinant HCV genotype 1a E2 protein (GenBank accession no. M62321) by phage display. The antibody was serially diluted and pre-incubated with 15,000 ffu of JFH-1 virus in a volume of 250 microliters for 1 hour at 37° C. The virus-antibody mixture was used to infect 45,000 Huh-7.5.1 cells in a 24-well plate (Corning) for 3 hours at 37° C.

Mouse monoclonal anti-human CD81 antibody 5A6 (Levy et al. (1998) Annu. Rev. Immunol. 16, 89-109) at a concentration of 1 mg/mL was serially diluted (1:2000, 1:200, 1:20) and pre-incubated in a volume of 50 μL with 10⁴ Huh-7.5.1 cells seeded into a 96-well plate for 1 hour at 37° C. Cells were subsequently inoculated with infectious JFH-1 supernatant at an moi of 0.3 for 3 hour at 37° C. The efficiency of the infection in the presence of antibodies was monitored 3 days post-infection by quantitative RT-PCR and immunofluorescence.

Interferon treatment. Subconfluent Huh-7.5.1 cells were pretreated 6 hours with 5, 50 and 500 IU/mL human IFNα-2a or IFNγ (PBL Biomedical Lab, Piscataway, N.J.) before inoculation with JFH-1 virus at an moi of 0.3. The inoculum was removed after 3 hours of incubation at 37° C., and fresh DMEM supplemented with the indicated doses of IFN was added to the cells. The efficiency of the infection was monitored 72 hours later by quantitative RT-PCR.

### Example 2

### Production of Infectious HCV Particles in Hepatoma Cultured Cells Transfected with HCV RNA

This Example illustrates that infectious HCV particles are efficiently produced when an HCV-negative Huh-7.5-derived cell line, referred to herein as Huh-7.5.1, is transfected with HCV RNA or cultured with supernatant from HCV RNA-transfected cells.

As described above, Huh-7.5.1 cells were derived from the Huh-7.5 GFP-HCV replicon cell line I/5A-GFP-6 (Moradpour (2004) J. Virol. 78, 7400-7409) by curing the HCV-GFP replicon from the I/5A-GFP-6 cells. To do this the I/5A-GFP-6 replicon cells were cultured for three weeks in the presence of 100 IU/mL human interferon gamma (IFNã). This eradicated the I/5A-GFP-6 replicon from the cells, thereby generating the Huh-7.5.1 cells. Clearance of the HCV replicon was confirmed by G418 sensitivity (the HCV replicon included a neomycin resistance gene) and by HCV-specific quantitative RT-PCR analysis.

Production of infectious HCV particles by Huh-7.5.1 hepatoma cells transfected with HCV RNA. In a first set of experiments, 10 μg of in vitro transcribed genomic JFH-1 RNA was delivered into Huh-7.5.1 cells by electroporation. Transfected cells were then passaged when necessary (usually about every 3-4 days) to maintain sub-confluent cultures throughout the experiment. At selected intervals, total RNA was isolated from the transfected Huh-7.5.1 cells and the level of HCV RNA was determined by HCV-specific quantitative RT-PCR. NS5A protein expression was also monitored by immunofluorescence and the release of infectious virus was determined by titration of transfected cell supernatants.

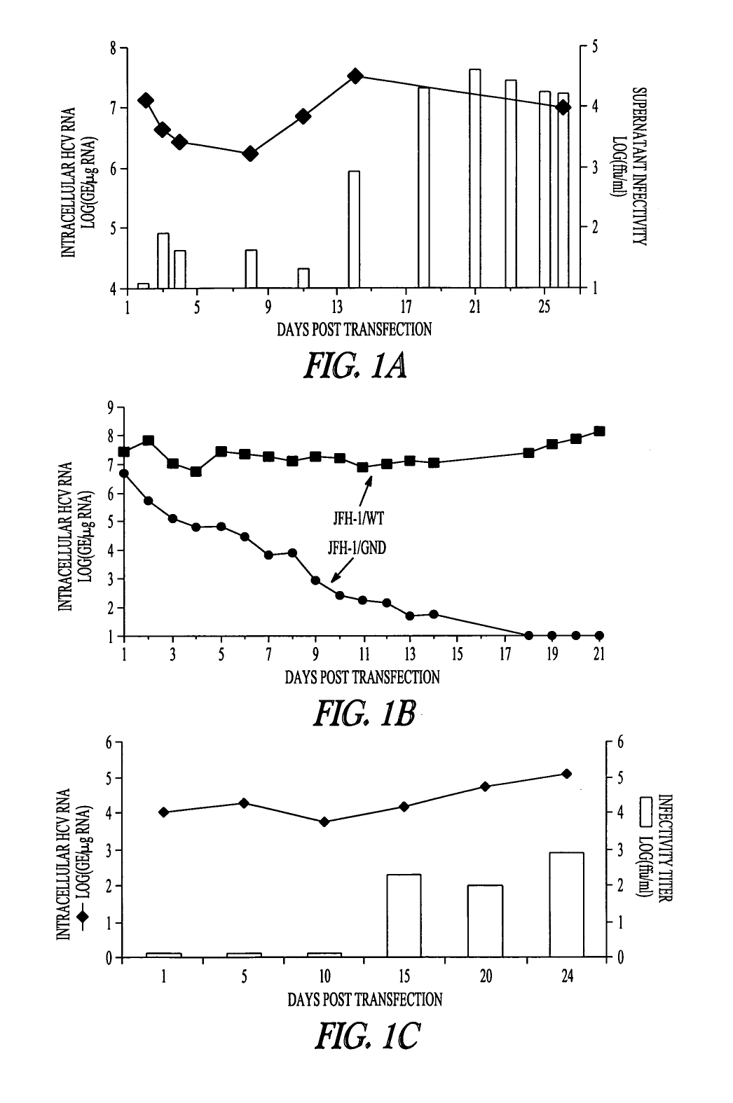

Two days post-transfection, 1.3×10⁷ copies of HCV RNA per μg of cellular RNA were detected (FIG. 1A), probably reflecting a combination of input RNA and RNA produced by intracellular HCV replication. HCV RNA levels subsequently decreased reaching a minimum level of 1.6'10⁶ copies per μg of cellular RNA at day 8 post-transfection (FIG. 1A). Importantly, however, intracellular HCV RNA levels began to increase thereafter, reaching maximal levels of more than 10⁷ copies per μg of total RNA by day 14 post-transfection, and these levels were maintained until the experiment was terminated on day 26 (FIG. 1A). These results indicated that HCV was actively replicating in transfected Huh-7.5.1 cells. This hypothesis is supported by a rapid disappearance of a replication-incompetent JFH-1 RNA genome after transfection (FIG. 1B).

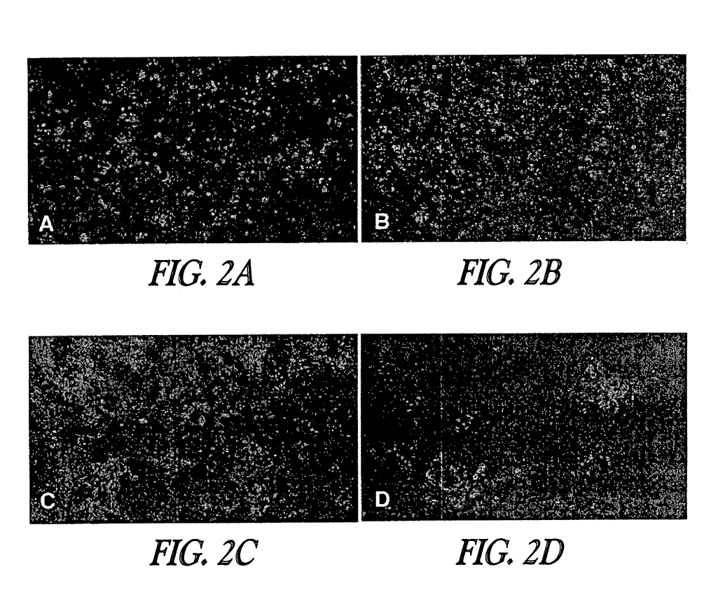

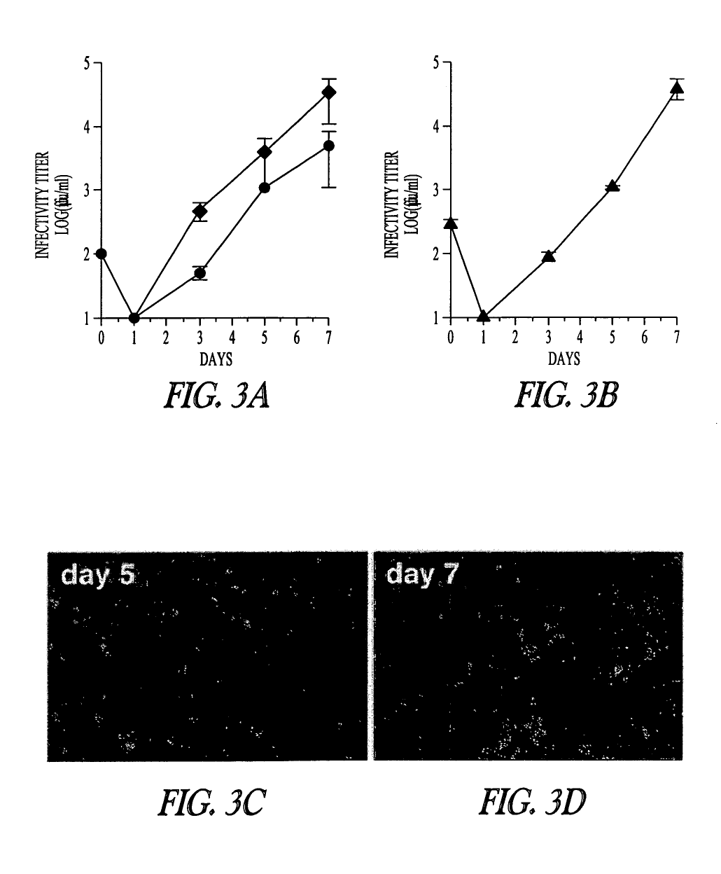

Interestingly, immunofluorescence staining for NS5A indicated that the percentage of NS5A positive cells in the transfected cell cultures increased from 2% on day 5 (FIG. 2A) to almost 100% on day 24 (FIG. 2B). These results were consistent with the amplification of HCV RNA, and further suggested that HCV transfected cells either had acquired a selective growth advantage or that HCV was spreading to untransfected cells within the culture.

To determine whether the JFH-1 transfected Huh-7.5.1 cells were releasing infectious virus, naïve Huh-7.5.1 cells were inoculated with supernatants collected at different time points during the transfection experiment. Immunofluorescence staining three days post-inoculation not only revealed NS5A positive cells in the culture (FIG. 2C), but when the supernatants were serially diluted, the infection resulted in discrete foci of NS5A-positive cells (FIG. 2D). Thus, the focus forming units per ml (ffu/mL) in the supernatants collected at different times post-transfection could be determined. This type of supernatant titration was performed for the transfection experiment described in FIG. 1A, and is indicated by vertical bars. Infectious virus was detected in the culture medium three days after transfection (80 ffu/mL), and then increased reaching a maximum of 4.6×10⁴ ffu/mL by day 21 post-transfection, concomitant with the amplification of intracellular JFH-1 RNA.

Taken together, these results indicate that Huh-7.5.1 cells transfected with genomic JFH-1 RNA were able to not only support HCV replication, but to also produce infectious HCV particles. Notably, similar results were obtained when JFH-1 RNA was delivered to Huh-7.5.1 cells by an alternative transfection method (i.e. liposomes; FIG. 1C).

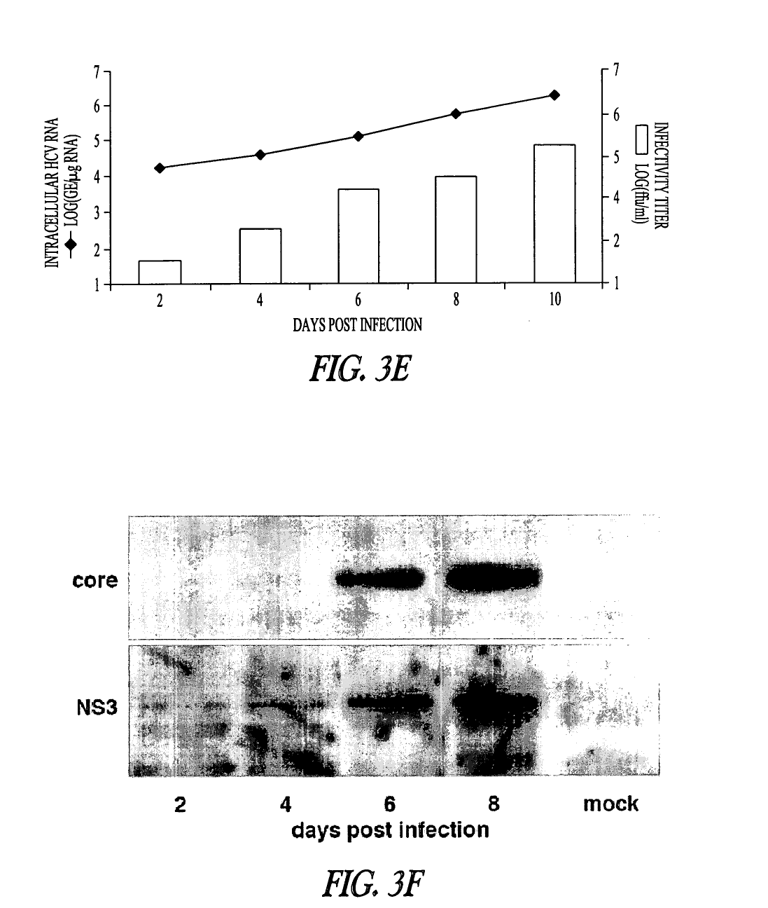

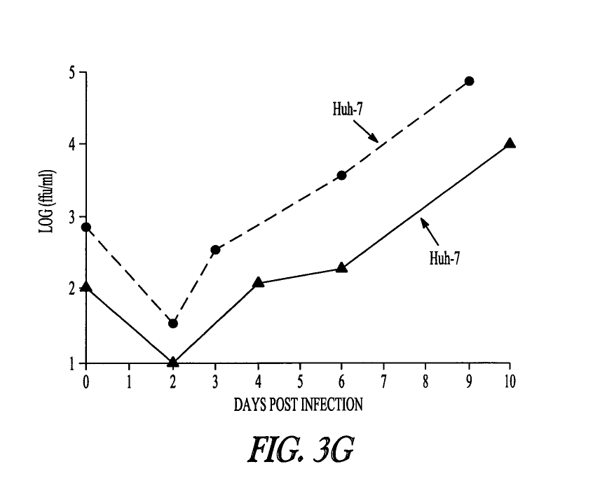

Propagation of HCV virus generated by transfection. Further experiments were performed to determine whether cells infected with JFH-1 transfected cell supernatant produced progeny virus that could be serially passaged to naïve Huh-7.5.1 cells. Naive Huh-7.5.1 cells were infected at low multiplicity of infection (MOI=0.01) with infectious supernatants collected from two independent transfection experiments and infectious virus production was monitored by titrating the infected cell supernatants at selected time points. On the first day after inoculation, no infectious particles were detectable in the supernatant of cells infected with either transfection cell inoculate (FIG. 3A). However, infectious particles exponentially accumulated in the supernatant thereafter reaching a maximal titer of at least 104 ffu/mL on day 7 after both infections (FIG. 3A). Thus, within 7 days post-infection in two separate experiments, HCV was amplified in naïve Huh-7.5.1 cells more than 100-fold. Similar kinetics were observed in the two separate transfection experiments.

In order to determine whether the progeny virus produced by infection, could be further passaged, naive Huh-7.5.1 cells were infected with the virus collected from one of the lipofection experiments (data from this lipofection experiment is shown in FIG. 3A). As shown in FIG. 3B, this secondary infection progressed with kinetics similar to that seen for the primary infection (FIG. 3A), again reaching maximal levels on day 7. The course of this secondary infection was reflected by increasing numbers of NS5A positive cells over the time course of the infection with almost all the cells being positive for NS5A at day 7 (FIG. 3C). These results indicate that the JFH-1 virus can be generated by transfection of JFH-1 RNA and the virions produced can be passaged in Huh-7.5.1 cells without a detectable loss in infectivity. Moreover, JFH-1 virions infect a high proportion of the cells in a relatively short period of time after introduction.

Additional experiments were also performed in which the intracellular levels of HCV RNA and proteins were monitored (FIG. 3E-F). This analysis confirmed that the appearance of infectious virus in the cell culture supernatant directly correlated with the amplification and subsequent translation of the input HCV RNA. Similar results were obtained for Huh-7 cells (FIG. 3G).

In sum, the virus produced in the cell supernatant by transfection could be serially passaged to naïve Huh-7 or Huh-7.5.1 cells. Infectious supernatant could infect naïve Huh-7 or Huh-7.5.1 cells at low multiplicity (MOI=0.01). The virus could propagate in the naïve cells and produce progeny viruses with kinetics similar to the primary infection. Furthermore, the progeny virus produced by infection could be further passaged to naïve cells without a detectable loss in infectivity. Thus, an important property of the in vitro infection system is that the virus produced in the cell supernatant by transfection can be serially passaged to naïve Huh-7 or Huh-7.5.1 cells.

HCV infection is inhibited by anti-E2 antibodies. HCV surface glycoprotein (E1/E2) pseudotyped viruses are described in Bartosch et al. (2003) J. Exp. Med. 197, 633-642; Hsu et al. (2003) Proc. Natl. Acad. Sci. U.S.A. 100, 7271-7276. Previous studies using these HCV surface glycoprotein (E1/E2) pseudotyped viruses have suggested that E1 and/or E2 mediate the interaction with cellular receptors that are required for viral adsorption. To verify whether such an interaction is required for HCV infection in vitro, neutralization experiments were performed using anti-E2 antibodies in which the JFH-1 virus was preincubated with serial dilutions of a recombinant human monoclonal antibody specific for HCV E2 or an isotype negative control antibody for 3 hours at 37° C. before infection.

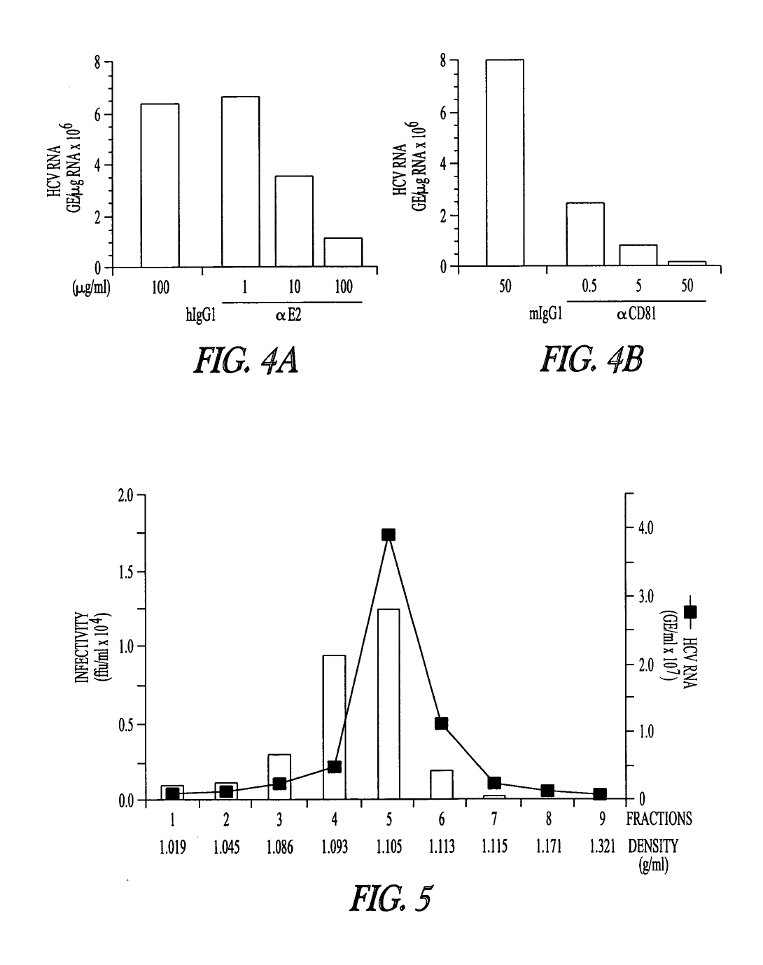

Huh-7.5.1 cells infected with JFH-1 virus (moi=0.3) in the presence of 100 μg/mL of anti-E2 antibody were found to have 5-fold lower intracellular HCV RNA levels compared to cells infected in the presence of the same amount of an isotype control antibody (FIG. 4A). The inhibition of HCV infection by anti-E2 antibodies was further reflected by a reduction in NS5A positive cells as determined by immunofluorescence (data not shown). Titration of the anti-E2 antibody indicated that 10 μg/mL of antibody was required for a 50% reduction in intracellular HCV RNA three days post-infection (FIG. 4A). These results are consistent with a conclusion that in vitro HCV infection in this system is partly mediated by the viral envelope E2 protein.

HCV infection is inhibited by anti-CD81 antibodies. Previous studies using pseudotyped viruses that express HCV E1/E2 have also suggested that the interaction between HCV E2 and CD81 is crucial for viral entry (Zhang et al. (2004) J. Virol. 78, 1448-1455). To determine whether CD81 is required in this HCV infection system, anti-CD81 antibody-pretreated naïve Huh-7.5.1 were infected with JFH-1 virus at an moi of 0.3 and analyzed 3 days post-infection. Intracellular HCV RNA levels were reduced in a dose dependent manner. In particular, a 50-fold reduction in HCV RNA was observed when 50 μg/mL anti-CD81 antibody was used compared to the control antibody-treated cells (FIG. 4B).

Biophysical properties of infectious HCV JFH-1 particles. To examine the density of the secreted infectious HCV virions, supernatants collected from uninfected and HCV-infected Huh7.5.1 cells were subjected to sucrose gradient centrifugation. Gradient fractions were collected after centrifugation, and analyzed for the presence of HCV RNA and infectivity (FIG. 5). Maximal infectivity titers (1.25×10⁴ ffu/mL) were present in fraction 5 and coincided with the peak of HCV RNA. The approximate 1.105 g/mL apparent density of the peak infectivity fraction was consistent with that previously reported for HCV virions isolated from patient sera (Hijikata et al. (1993) J. Virol. 67, 1953-1958; Trestard et al. (1998) Arch. Virol. 143, 2241-2245). These data indicate that the density of the recombinant JFH-1 virus is similar to that of HCV isolated from humans.

In vitro tropism of JFH-1 HCV To determine whether infection with the JFH-1 virus was restricted to Huh-7.5.1 cells, attempts were made to infect a panel of hepatic (Huh-7 and HepG2) and non-hepatic cell lines (HeLa, HEK293, HL-60, U-937 and EBV-transformed B cells). Besides the Huh-7.5.1 cells, only the Huh-7 cells were permissive for HCV infection as determined by immunofluorescent staining for the viral NS5A protein at day 3 post-infection (data not shown).

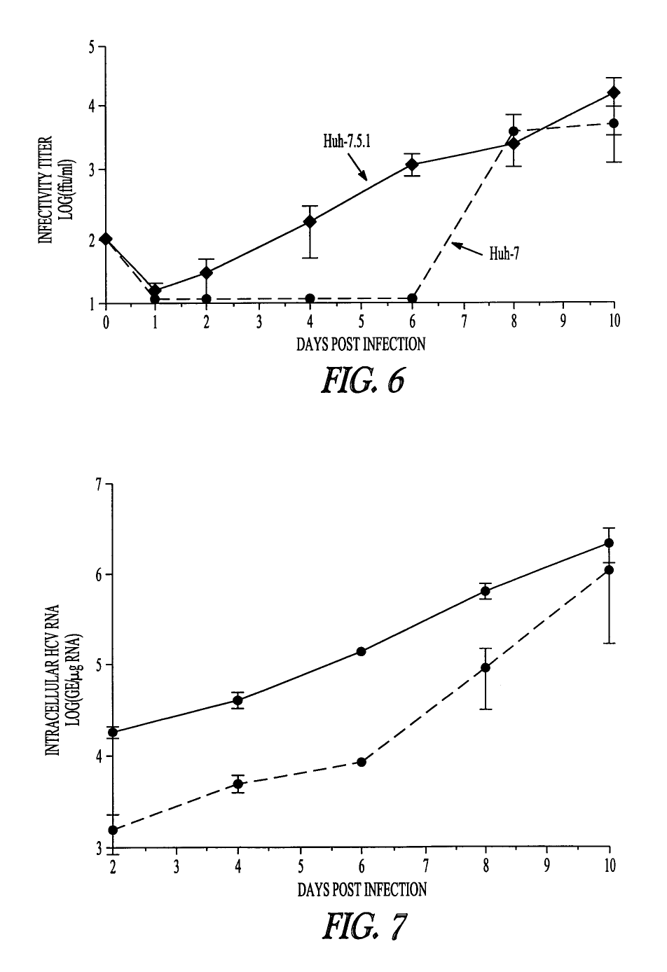

To determine whether there were quantitative differences in infection efficiency between the Huh-7.5.1 and Huh-7 cells, both cell lines were infected in parallel. As shown in FIG. 6, infectious particle release into the supernatant of infected Huh-7 cells appeared to be delayed when compared to the particle production by Huh-7.5.1 cells. Nevertheless, Huh-7 cells produced similar amounts of infectious particles by day 8 and 10. Similar delayed kinetics in the amplification of intracellular HCV RNA was also observed in the Huh-7 cells (FIG. 7). These results demonstrate that Huh-7 cells can produce similar amounts of progeny virus as Huh-7.5.1 cells, but with delayed kinetics.

The results reported herein indicate that JFH-1 -transfected or infected Huh-7.5.1 cells constitute a simple, yet robust, cell culture system for HCV infection, which allows the rescue of infectious virus from the JFH-1 consensus cDNA clone. Thus, as illustrated herein, transfection of JFH-1 RNA into the Huh-7-derived cells allows for the recovery of viable JFH virus that can then be serially passaged and used for infection-based experimentation. Impressively, infection with serial dilutions of the virus resulted in the formation of infected cell foci that allowed us to quantitatively titrate the HCV being produced.

Thus, the disappearance of input virus from the supernatant within 24 hours post infection indicates that virus particles were able to enter the cells within this time frame. As infectious viral titers rose from these undetected levels to 10⁴ to 10⁵ ffu/mL, the number of NS5A positive cells also increased, suggesting that the virus was spreading to new cells (FIG. 3C). Importantly, when passaged to naïve Huh-7.5.1 cells, the virus produced by both transfected and infected cells exhibited the same infection kinetics with an HCV doubling time of approximately 22 hours. This doubling time is longer than the 6 to 8 hours previously reported in infected patients (Buhk et al. (2002) Proc. Natl. Acad. Sci. USA 99: 14416-21) and chimpanzees (Neumann et al. (1998) Science 282: 103-107), however, technical and biological factors may be responsible for this discrepancy. For example, the earlier estimates were based on the number of HCV genome equivalents detected in the serum of infected individuals, not the infectivity titer observed as in the current study.

The fact that an antibody directed against the viral surface glycoprotein E2 reduced the infectivity of the JFH-1 virus, suggests that the process of viral adsorption and entry can be studied in this system. Consistent with this assertion, HCV infection of Huh-7-derived cells was inhibited by an antibody against CD81 (FIG. 4), an extensively characterized putative HCV receptor.

The tropism of the JFH-1 virus, thus far appears to be limited to Huh-7-derived cell lines. Previous work has shown that HepG2, HeLa, and HEK293 cells support replication of the subgenomic JFH-1 replicon. See Blight et al. (2000) Science 290, 1972-1975; Kato et al. (2001) J. Med. Virol. 64, 334-339; Date et al. (2004) J. Biol. Chem. 279, 22371-22376. However, the HepG2, HeLa, and HEK293 cells failed to become infected with JFH-1 virions as described above. In contrast, non-HCV adapted Huh-7 cells were found to be susceptible to infection with the JFH-1 virus (FIG. 6). Virus amplification in Huh-7 cells was somewhat slower, but the Huh-7 cells eventually produced viral titers comparable to those attained in Huh-7.5.1 cells.

Huh7.5 cells contain an inactivating mutation in RIG-I (Neumann et al. (1998) Science 282, 103-107), which is a key component of the cellular double-stranded RNA sensing machinery (Tanaka et al. (2005) Intervirology 48, 120-123). It appears that HCV infection may induce a double-stranded RNA antiviral defense pathway in Huh-7 cells, which transiently delays viral replication and/or spread. The fact that HCV eventually overcomes the limitations present in Huh-7 cells and reaches titers similar to those produced by Huh-7.5.1 cells further suggests that expression of one or more viral encoded functions (e.g. NS3, NS5A) may block or negate the intracellular antiviral defense(s). HCV infection, however, remained sensitive to the effects of exogenously added interferon—both IFNα and IFNγ prevented JFH-1 virus infection of Huh-7.5.1 cells (FIG. 8). Interestingly, these in vitro observations appear to parallel those seen clinically, where interferon therapy is able to reduce viral titers in some patients regardless of the mechanisms the virus has evolved to allow it to persist in the presence of the IFN it induces.

Thus, a robust cell culture model of HCV infection has been established in which infectious HCV can be produced and serially passaged to naïve cells.

### Example 3

### HCV Peptides Inhibit Hepatitis C Viral Infection

As described above, Huh-7 and Huh-7.5.1 cells can be infected in vitro with virus produced by an HCV genotype 2a JFH-1 clone. This Example illustrates that HCV peptides having SEQ ID NO:6, 8, 12, 13, 14, 24, 27, 30, 32, 43, 44, 47, 48 and 53 strongly inhibit HCV infection as measured using this cell culture model of HCV infection described above. Other peptides exhibited good inhibition of HCV infection. These HCV-derived synthetic peptides that were effective inhibitors were from both structural and non-structural regions of the HCV polyprotein.

A peptide library of 441 overlapping peptides covering the complete HCV polyprotein of genotype 1a (H77) (SEQ ID NO:1) was tested. The peptides were about 18 amino acids in length with 11 overlapping amino acids. The peptide library was provided by NIH AIDS Research and Reference Reagent Program (Cat #7620, Lot #1).

To identify peptides that display antiviral activity against HCV infection, the peptide library was screened by an HCV focus reduction assay. The peptides were constituted in 100% DMSO at a final concentration 10 mg/mL, and stored in −20° C. The peptide stock solution was diluted 1:200 to a final concentration approximately 20 μM in complete DMEM growth medium containing 50 focus forming units (ffu) of HCV. The virus-peptide mixture was transferred to Huh-7.5.1 cells at a density of 8000 cells per well in a 96-well plate. After adsorption for 4 hours at 37° C., the inoculum was removed. The cells were washed 2 times, overlaid with 120 μL fresh growth medium and incubated at 37° C. After 3 days of culture, the cells were fixed with paraformaldehyde and immunostained with antibody against HCV nonstructural protein NS5A. The numbers of HCV foci were counted under fluorescent microscopy and the result is expressed as percentage (%) of mock with no peptide treatment but containing solvent 0.5% DMSO.

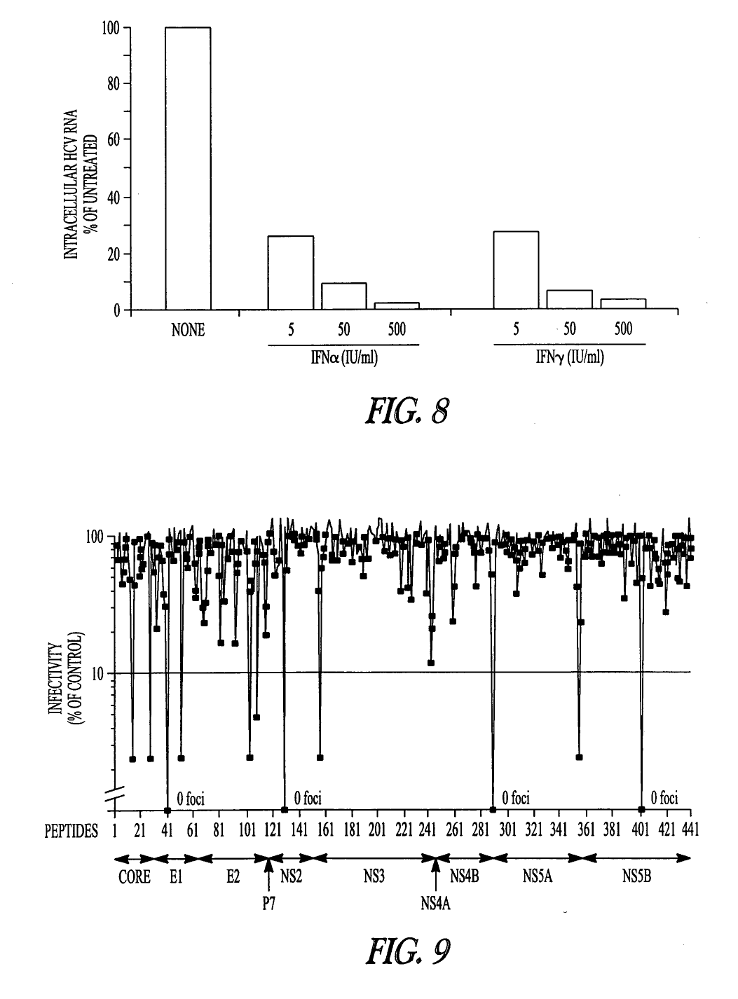

The results of these assays are shown in FIG. 9 and the following table.

TABLE 3
Inhibition of HCV Infection

| | | | | | | | |
|---|---|---|---|---|---|---|---|
| | | | | | | | SEQ |
| | Peptide | % of | Fold | >10- | 5-10 | 2-5 | ID |
| No. | Sequence | Mock | Inhibition | fold | fold | fold | NO: |

| | | | | | | | |
|---|---|---|---|---|---|---|---|
| 6930 | QIVGGVYLLPR | 45.2 | 2.2 | | | * | 4 |
| | RGPRLGV | | | | | | |
| 6937 | QPGYPWPLYGN | 50.0 | 2.0 | | | * | 5 |
| | EGCGWAG | | | | | | |
| 6938 | LYGNEGCGWAG | 2.4 | 42.0 | *** | | | 6 |
| | WLLSPRG | | | | | | |
| 6939 | GWAGWLLSPRG | 45.2 | 2.2 | | | * | 7 |
| | SRPSWGP | | | | | | |
| 6951 | IFLLALLSCLT | 2.4 | 42.0 | *** | | | 8 |
| | VPASAYQ | | | | | | |
| 6957 | DAILHTPGCVP | 21.4 | 4.7 | | | * | 9 |
| | CVREGNA | | | | | | |
| 6962 | LPTTQLRRHID | 38.1 | 2.6 | | | * | 10 |
| | LLVGSAT | | | | | | |
| 6963 | RHIDLLVGSAT | 31.0 | 3.2 | | | * | 11 |
| | LCSALYV | | | | | | |
| 6964 | GSATLCSALYV | 1.0 | 100.0 | *** | | | 12 |
| | GDLCGSV | | | | | | |
| 6965 | ALYVGDLCGSV | 1.0 | 100.0 | *** | | | 13 |
| | FLVGQLF | | | | | | |
| 6975 | IMDMIAGAHWG | 2.4 | 42.0 | *** | | | 14 |
| | VLAGIAY | | | | | | |
| 6986 | HINSTALNCNE | 40.5 | 2.5 | | | * | 15 |
| | SLNTGWL | | | | | | |
| 6987 | NCNESLNTGWL | 35.7 | 2.8 | | | * | 16 |
| | AGLFYQH | | | | | | |
| 6991 | LASCRRLTDFA | 35.7 | 2.8 | | | * | 17 |
| | QGWGPIS | | | | | | |
| 6992 | TDFAQGWGPIS | 31.0 | 3.2 | | | * | 18 |
| | YANGSGL | | | | | | |
| 6993 | GPISYANGSGL | 23.8 | 4.2 | | | * | 19 |
| | DERPYCW | | | | | | |
| 6994 | GSGLDERPYCW | 33.3 | 3.0 | | | * | 20 |
| | HYPPRPC | | | | | | |
| 7005 | WMNSTGFTKVC | 16.7 | 6.0 | | ** | | 21 |
| | GAPPCVI | | | | | | |
| 7007 | PCVIGGVGNNT | 33.3 | 3.0 | | | * | 22 |
| | LLCPTDC | | | | | | |
| 7016 | MYVGGVEHRLE | 16.7 | 6.0 | | ** | | 23 |
| | AACNWTR | | | | | | |
| 7026 | YLYGVGSSIAS | 2.4 | 42.0 | *** | | | 24 |
| | WAIKWEY | | | | | | |
| 7027 | SIb 11541488.3 | | | | | | |
| | ASWAIKWEY | 40.5 | 2.5 | | | * | 25 |
| | VVLLFLL | | | | | | |
| 7028 | KWEYVVLLFLL | 47.6 | 2.1 | | | * | 26 |
| | LADARVC | | | | | | |
| 7031 | WMMLLISQAEA | 4.8 | 21.0 | *** | | | 27 |
| | ALENLVI | | | | | | |
| 7038 | GAVYAFYGMWP | 19.0 | 5.3 | | ** | | 28 |
| | LLLLLLA | | | | | | |
| 7039 | GMWPLLLLLLA | 31.0 | 3.2 | | | * | 29 |
| | LPQRAYA | | | | | | |
| 7052 | TLVFDITKLLL | 1.0 | 100.0 | *** | | | 30 |
| | AIFGPLW | | | | | | |
| 7725 | VSTATQTFLAT | 40.5 | 2.5 | | | * | 31 |
| | CIN | | | | | | |
| 7078 | ATQTFLATCIN | 2.4 | 42.0 | *** | | | 32 |
| | GVCWTVY | | | | | | |
| 7142 | DSSVLCECYDA | 40.5 | 2.5 | | | * | 33 |
| | GCAWYEL | | | | | | |
| 7146 | AYMNTPGLPVC | 40.5 | 2.5 | | | * | 34 |
| | QDHLEFW | | | | | | |
| 7148 | LEFWEGVFTGL | 33.3 | 3.0 | | | * | 35 |
| | THIDAHF | | | | | | |
| 7160 | HPITKYIMTCM | 38.1 | 2.6 | | | * | 36 |
| | SADLEVV | | | | | | |
| 7729 | TSTWVLVGGVL | 11.9 | 8.4 | | ** | | 37 |
| | AAL | | | | | | |
| 7163 | WVLVGGVLAAL | 26.2 | 3.8 | | | * | 38 |
| | AAYCLST | | | | | | |
| 7730 | LAALAAYCLST | 21.4 | 4.7 | | | * | 39 |
| | GCVV | | | | | | |
| 7177 | EVFWAKHMWNF | 23.8 | 4.2 | | | * | 40 |
| | ISGIQYL | | | | | | |
| 7178 | MWNFISGIQYL | 42.9 | 2.3 | | | * | 41 |
| | AGLSTLP | | | | | | |
| 7195 | PAILSPGALVV | 42.9 | 2.3 | | | * | 42 |
| | GVVCAAI | | | | | | |
| 7208 | SWLRDIWDWIC | 1.0 | 100.0 | *** | | | 43 |
| | EVLSDFK | | | | | | |
| 7209 | DWICEVLSDFK | 2.4 | 42.0 | *** | | | 44 |
| | TWLKAKL | | | | | | |
| 7226 | YVSGMTTDNLK | 38.1 | 2.6 | | | * | 45 |
| | CPCQIPS | | | | | | |
| 7740 | SSGADTEDVVC | 42.9 | 2.3 | | | * | 46 |
| | CSMS | | | | | | |
| 7741 | DTEDVVCCSMS | 2.4 | 42.0 | *** | | | 47 |
| | YSW | | | | | | |
| 7270 | SSGADTEDVVC | 4.5 | 22.0 | *** | | | 48 |
| | CSMSYSW | | | | | | |
| 7742 | DVVCCSMSYSW | 23.8 | 4.2 | | | * | 49 |
| | TGAL | | | | | | |
| 7304 | TVTESDIRTEE | 35.7 | 2.8 | | | * | 50 |
| | AIYQCCD | | | | | | |
| 7313 | GNTLTCYIKAR | 45.2 | 2.2 | | | * | 51 |
| | AACRAAG | | | | | | |
| 7315 | RAAGLQDCTML | 50.0 | 2.0 | | | * | 52 |
| | VCGDDLV | | | | | | |
| 7316 | CTMLVCGDDLV | 1.0 | 100.0 | *** | | | 53 |
| | VICESAG | | | | | | |
| 7317 | DDLVVICESAG | 26.2 | 3.8 | | | * | 54 |
| | VQEDAAS | | | | | | |
| 7323 | LELITSCSSNV | 42.9 | 2.3 | | | * | 55 |
| | SVAHDGA | | | | | | |
| 7329 | HTPVNSWLGNI | 47.6 | 2.1 | | | * | 56 |
| | IMFAPTL | | | | | | |
| 7331 | APTLWARMILM | 45.2 | 2.2 | | | * | 57 |
| | THFFSVL | | | | | | |
| 7334 | DQLEQALNCEI | 28.6 | 3.5 | | | * | 58 |
| | YGACYSI | | | | | | |
| 7342 | GVPPLRAWRHR | 50.0 | 2.0 | | | * | 59 |
| | ARSVRAR | | | | | | |
| 7343 | WRHRARSVRAR | 47.6 | 2.1 | | | * | 60 |
| | LLSRGGR | | | | | | |
| 7350 | GWFTAGYSGGD | 42.9 | 2.3 | | | * | 61 |
| | IYHSVSH | | | | | | |
| | Total | | | 14 | 4 | 41 | |

Of the 441 peptides, 382 had no effect on HCV infection or blocked it by less than 20% (not shown in Table 3). Forty-one peptides slightly inhibited HCV infection by about 2- to 5-fold. Four peptides inhibited HCV infection by about 5- to 10-fold. Fourteen peptides inhibited HCV infection by more than 10-fold. In particular, HCV infection was profoundly inhibited (90-100%) by peptides with SEQ ID NO:6, 8, 12, 13, 14, 24, 27, 30, 32, 43, 44, 47, 48 and 53. No evidence of toxicity was detected when Huh-7.5.1 cells were incubated with these peptides. These results identify peptide inhibitors that may modify or inhibit one or more steps in the viral life cycle. Moreover, according to the invention, these peptides can be used in antiviral compositions and methods for inhibiting HCV infection. Peptides that inhibited infection by more than 90% were selected for further analysis.

To accurately quantify the inhibitory effect of the selected peptides on HCV infection, intracellular HCV RNA was measured after infection by real time RT-QPCR with and without peptide treatment. The peptide stock solution was diluted 1:100 and mixed with equal volume of viral supernatant (propagated from day 18 virus preparation post transfection) to a final concentration approximately 20 μM. The virus with peptide or 0.5% DMSO solvent control was then used to infect Huh-7.5.1 cells at a multiplicity of infection (MOI) of 0.1. After an adsorption for 4 hours at 37° C., the inoculum was removed. The cells were washed 2 times, overlaid with 120 μL fresh growth medium and incubated at 37° C. At the indicated time points, total cellular RNA was isolated by the guanidine thiocyanate method. The HCV RNA transcript level was measured by real time RT-QPCR with the primers 5′—

| | |
|---|---|
| TCTGCGGAACCGGTGAGTA-3′ | (sense, SEQ ID NO: 89) |
| and | |
| 5′-TCAGGCAGTACCACAAGGC-3′, | (antisense, |
| and | SEQ ID NO: 90)′ |

normalized to cellular GAPDH levels. Results are summarized in the following table.

TABLE 4
Inhibitory Peptide Hierarchy

HCV RNA
fold
inhibition

| | | | | | |
|---|---|---|---|---|---|
| | | Amino Acid | SEQ | 24 h | 72 h |
| Peptide | Peptide | Sequence | ID | 25 μM | 25 μM |

| | | | | | |
|---|---|---|---|---|---|
| 1 | NS5A | SWLRDIWDWICEV | 43 | 1,860 | 245,658 |
| | 1975 | LSDFK | | | |
| | Membrane | | | | |
| | Anchor | | | | |
| 2 | NS5B | CTMLVCGDDLVVI | 53 | 86 | 40 |
| | 2731 | CESAG | | | |
| | Catalytic | | | | |
| | Domain | | | | |
| 3 | NS5A/5B | SSGADTEDVVCCS | 48 | 75 | 49 |
| | 2413 | MSYSW | | | |
| | “BILN | | | | |
| | 2061” | | | | |
| 4 | E2/P7 736 | WMMLLISQAEAAL | 27 | 27 | 40 |
| | | ENLVI | | | |
| 5 | E1 267 | GSATLCSALYVGD | 12 | 26 | 27 |
| | Putative | LCGSV | | | |
| | fusion | | | | |
| | peptide | | | | |
| 6 | NS2 883 | TLVFDITKLLLAI | 30 | 24 | 16 |
| | | FGPLW | | | |
| 7 | E1 274 | ALYVGDLCGSVFL | 13 | 20 | 11 |
| | Putative | VGQLF | | | |
| | fusion | | | | |
| | peptide | | | | |
| 8 | Core/E1 | IFLLALLSCLTVP | 8 | 18 | 7 |
| | 176 Signal | ASAYQ | | | |
| | Peptide | | | | |
| | cleavage | | | | |
| 9 | E1 344 | IMDMIAGAHWGVL | 14 | 10 | 22 |
| | | AGIAY | | | |
| 10 | Core 85 | LYGNEGCGWAGWL | 6 | 9 | 11 |
| | | LSPRG | | | |
| 11 | E2 701 | YLYGVGSSIASWA | 24 | 9 | 7 |
| | | IKWEY | | | |
| 12 | NS3 1065 | ATQTFLATCINGV | 32 | 8 | 5 |
| | | CWTVY | | | |
| 13 | NS5A | DWICEVLSDFKTW | 44 | 7 | 2 |
| | 1982 | LKAKL | | | |
| | Membrane | | | | |
| | Anchor | | | | |

Based on the hierarchy of infectivity, most active peptides were reassigned numerical designators to reflect their position in the hierarchy of infectivity as shown in the above Table.

### Example 4

### Analyses of N— and C-terminal Truncated Peptide 1

To define the antiviral action of peptide #1 (SEQ ID NO:43), the antiviral activity of a series of N-terminal and C-terminal truncations of peptide 1 was analyzed using the focus reduction assay and by measuring the reduction in intracellular HCV RNA as described.

Highly purified peptides (>95% purity) were used for these studies. All peptides were synthesized using fluorenylmethoxycarbonyl (Fmoc) chemistry on pre-loaded wang resin by A & A Labs, LLC (San Diego, Calif.). The peptides were synthesized on the Symphony multiple peptide synthesizer (Protein Technologies Inc, Tucson, Ariz.). The crude peptides were then purified and analyzed by reverse-phase Gilson HPLC system (Gilson, Inc. Middleton, Wis.). The column used was C18 column (Grace Vydac, Hesperia, Calif.) with bead size 20 mm and length 250 mm. The solvent system was a H₂O and acetonitrile solvent system with a linear gradient of 5% to 70% for 30 minutes. Mass spectral analysis was performed by PE Sciex API-100 mass spectrometer. This confirmed the molecular masses of the synthesized peptides. Peptide concentration was determined using the extinction coefficient of the chromophore residues (Tryptophan or Tyrosine), where Tryptophan=5560 AU/mmole/mL and Tyrosine 1200=AU/mmole/mL. Calculations were made using the formula:mg peptide per mL=(A₂₈₀×DF×MW)/e, where A₂₈₀ was the actual absorbance of the solution at 280 nm in a 1-cm cell, DF was the dilution factor, MW was the molecular weight of the peptide and e was the molar extinction coefficient of each chromophore at 280 nm.

Results, summarized in the following table, show that the peptides having C-terminal truncations of 1 to 4 amino acid residues retained antiviral activity. Removal of as few as 2 amino acids from the N-terminus destroyed antiviral activity.

TABLE 5
Anti-HCV Activity of Truncated Variants of Peptide 1

| | | | | | |
|---|---|---|---|---|---|
| | | | | Fold | Fold |
| | | | | Inhibition | Inhibition |
| | | Working | Focus | on HCV | on HCV |
| | SEQ | Concen- | Reduction | RNA | RNA |
| Peptides | ID | tration | (72 h) | (24 h) | (72 h) |

| | | | | | |
|---|---|---|---|---|---|
| SWLRDIWDWICEVLSDFK | 43 | 19.5 μM | 0 | 47697.9 | 301779.7 |
| SWLRDIWDWICEVLSD | 94 | 18.4 μM | 0 | 9852.6 | 234207.1 |
| SWLRDIWDWICEVL | 92 | 23.6 μM | 0 | 20274.7 | 237172.4 |
| SWLRDIWDWICE | 104 | 21.4 μM | 107 | 0.8 | 0.5 |
| SWLRDIWDWI | 105 | 25.6 μM | 65 | 0.8 | 0.6 |
| SWLRDIWD | 106 | 24.7 μM | 58 | 1.3 | 1.0 |
| LRDIWDWICEVLSDFK | 107 | 18.6 μM | 125 | 0.7 | 0.6 |
| DIWDWICEVLSDFK | 108 | 27.1 μM | 125 | 1.0 | 0.7 |
| WDWICEVLSDFK | 109 | 24.7 μM | 38 | 2.2 | 1.4 |
| WICEVLSDFK | 110 | 27.5 μM | 45 | 1.1 | 1.0 |
| CEVLSDFK | 111 | NA | 53 | 1.1 | 0.7 |
| Mock | | | 51 | | |

### Example 5

### Anti-HCV Activity of Peptide 1

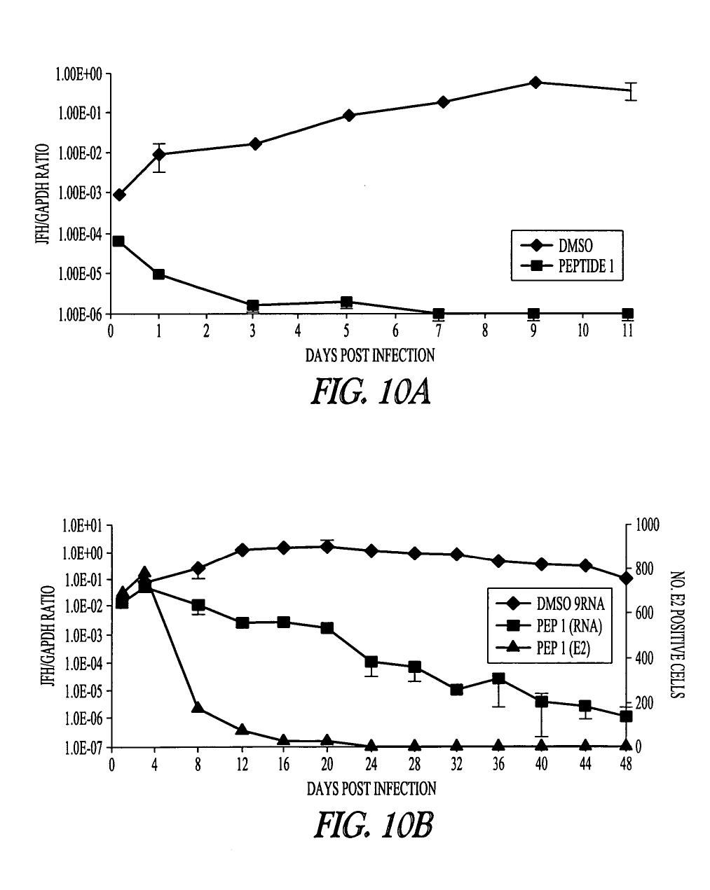

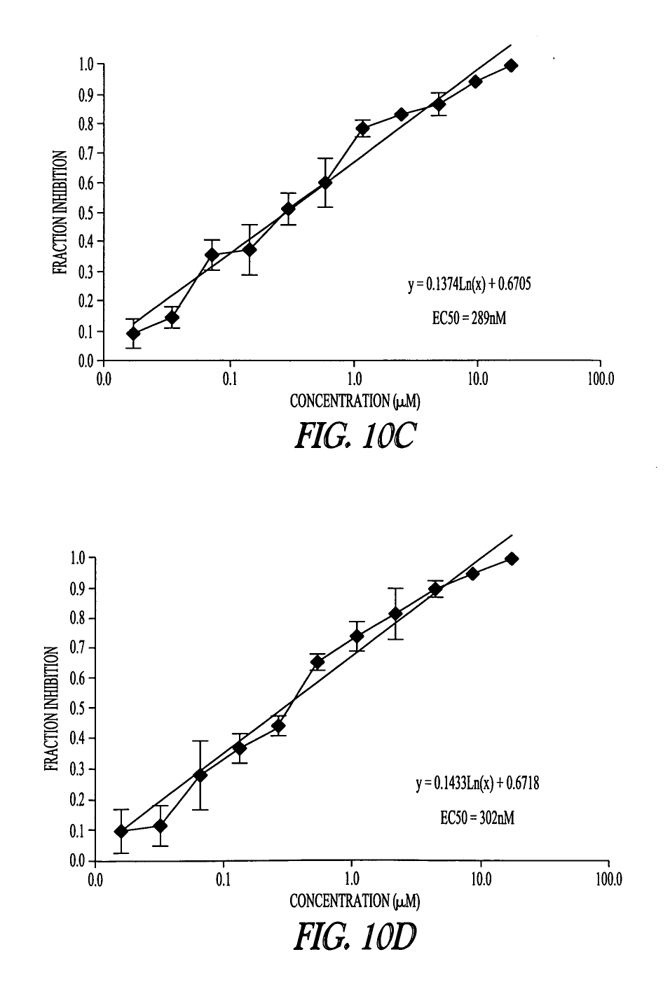

To examine the duration of the antiviral effect of peptide #1, Huh-7.5.1 cells were infected with HCV at an MOI=0.1 with a single dose of peptide #1 at 18 μM. After 4 hours at 37° C., the virus-peptide inoculum was removed. The cells were washed 2 times, overlaid with 120 μL of fresh growth medium and incubated at 37° C. Cells were split at a ratio of 6 when reaching confluency and maintained for 11 days. At the indicated time points, total cellular RNA was isolated by the guanidine thiocyanate method. HCV RNA transcript level was measured by real time RT-QPCR and normalized to cellular GAPDH levels. The results (FIG. 10A) show that peptide 1 permanently prevents HCV infection.

To determine if peptide #1 could abolish ongoing infection, Huh-7 cells were first infected with HCV at an MOI=0.1. After an adsorption for 4 hours at 37° C., the virus inoculum was removed. The cells were then washed 2 times by growth medium and overlaid with 120 μL fresh medium containing either peptide #1 at 18 μM or 0.5% DMSO as control, and the peptide was maintained in the culture medium thereafter. The cells were incubated at 37° C. until confluency, at which point they were split at a ratio of 1:4. When splitting, part of the cell suspension was subjected to RNA analysis. Total cellular RNA was isolated by the guanidine thiocyanate method. The HCV RNA transcript level was measured by real time RT-QPCR and normalized to cellular GAPDH levels. In parallel, cells were immunostained with antibody against HCV E2 protein and the number of HCV E2 positive cells were counted under fluorescent microscope. The results (FIG. 10B) demonstrate that adding peptide #1 at 4 hours after infection and maintaining it in the culture medium had no effect on the first round of viral amplification since viral infectivity titers and intracellular viral RNA were the same in all groups until the cells were split on day 4. However, by adding the peptide to the cultures each time the cells were split, further viral amplification (square) was prevented by rapidly and profoundly reducing supernatant infectivity titers (triangle).

To determine the median effective concentration (EC₅₀) of peptide #1, peptide stock solution (3.6 mM in DMSO) was serially 2-fold diluted in DMSO. An aliquot of peptide from each dilution was then diluted 1:100 in complete growth medium and mixed with equal volume of virus supernatant. The virus-peptide mixture was then used to infect Huh-7.5.1 cells (MOI=0.1). After adsorption for 4 hours at 37° C., the virus-peptide inoculum was removed. The cells were washed 2 times, overlaid with 120 μL fresh growth medium and incubated at 37° C. for 3 days. Cells were lysed and subjected to RNA analysis. The HCV RNA transcript level was measured by real time RT-QPCR and normalized to cellular GAPDH levels. The inhibition of HCV infection was calculated by comparing the intracellular HCV RNA transcript between the peptide treatment and solvent control. The results (FIG. 10C-D) show that the EC₅₀ of peptide #1 is approximately 300 nM under these conditions.

### Example 6

### Determination of the Mechanism of Antiviral Activity of Peptide #1

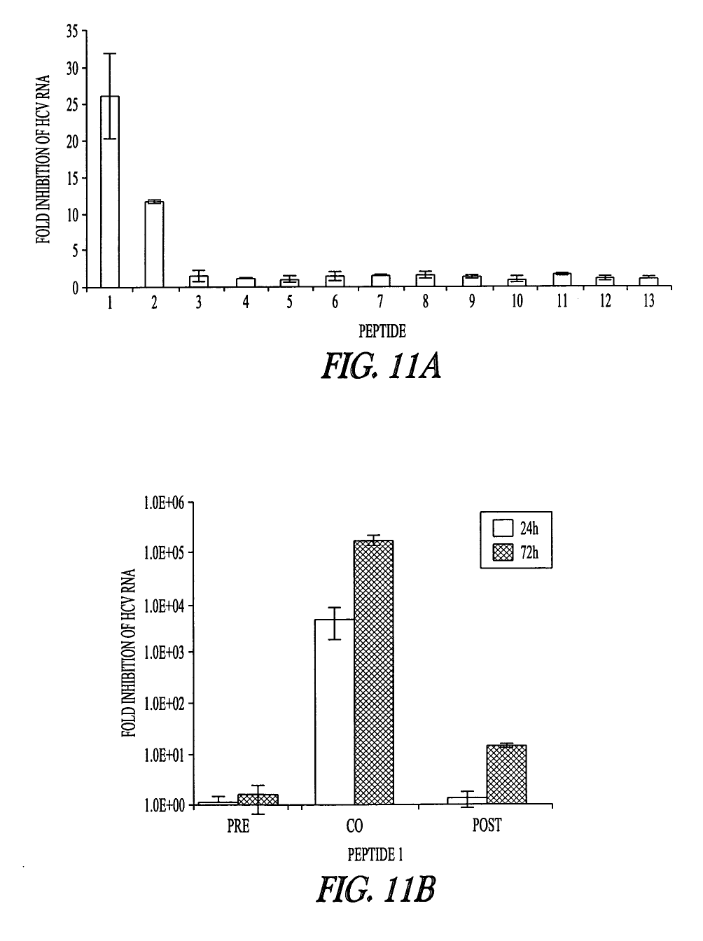

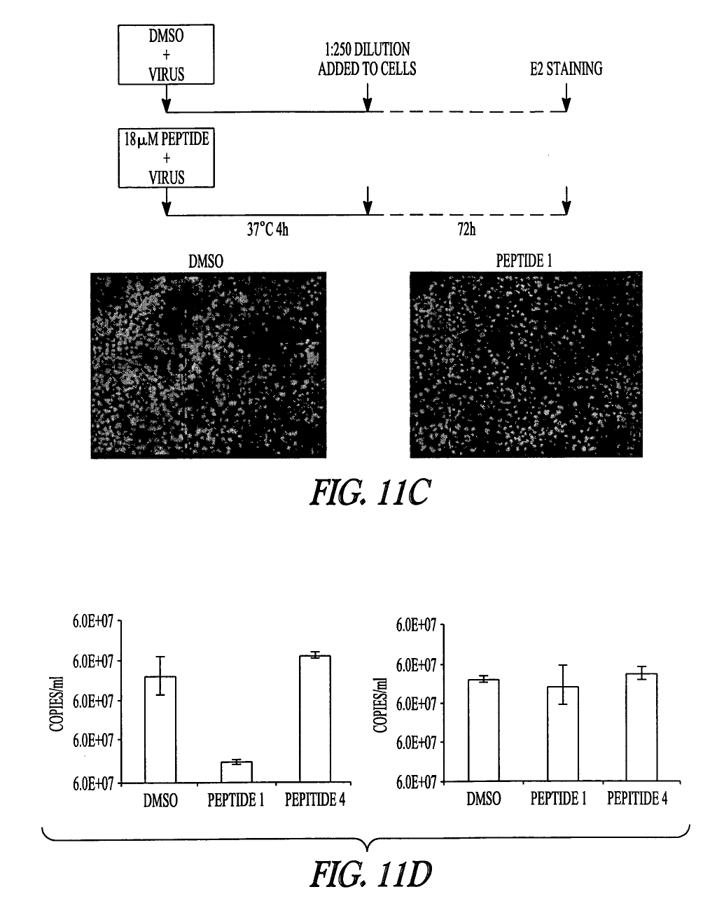

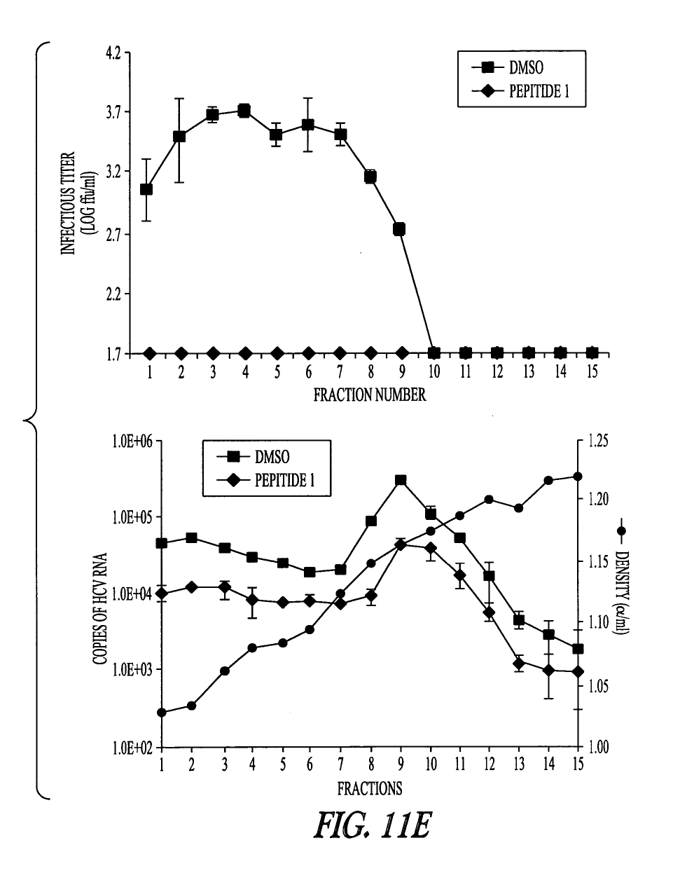

To define the mechanism of antiviral activity of peptide #1, its ability to prevent the binding/attachment/uptake by cells of viral RNA in an infectious inoculum cells was examined. Huh-7.5.1 cells were seeded at 8000 cells per well in a 96-well plate. Sixteen hours later, the cells were incubated with HCV at MOI=0.1 in the presence or absence of peptide at a concentration of 18 μM. After adsorption for 4 hours at 37° C., the virus-peptide inoculum was removed. The cells were washed 2 times, lysed and subjected to RNA analysis. The HCV RNA transcript level was measured by real time quantitative polymerase chain reaction (RT-QPCR) assay and normalized to cellular GAPDH levels. Inhibitory activity was quantified by comparing the amount of cell-associated HCV RNA in cells exposed to the virus-peptide inocula versus the virus-DMSO control. The results (FIG. 11A) indicate that peptide 1 (and peptide 2, which overlaps with peptide 1) significantly blocks viral binding/attachment/uptake while none of other peptides are active at this level.

To further define the mechanism of action, peptide #1 was added to the cells at different times relative to the time of addition of the inoculum. Huh-7.5.1 cells were seeded at 8000 cells per well in a 96-well plate. After overnight growth, the cell monolayer was infected with 8000 ffu/well of HCV. Peptide #1 was added to a final concentration 18 μM at three different times: 1) pre-inoculation (i.e. 4 hour incubation with cells followed by washing before virus infection); 2) co-inoculation (i.e. concurrent with the virus for 4 hours after which the virus and peptide were removed by washing); 3) post-inoculation (i.e. virus was added for 4 hours, and then the cells were washed to remove virus, and peptide was added and maintained for the duration of the experiment). At 24 hours and 72 hours post-infection, cells were lysed and subjected RNA analysis. The HCV RNA transcript level was measured by real time RT-QPCR and normalized to cellular GAPDH levels. The results (FIG. 11B) indicate that the peptide was most effective when it was added together with the virus, and thus, direct viral neutralization as the most likely mechanism of action.

Peptide #1 could be virocidal to HCV virions or block the interaction between the virus and cells. To further elucidate the mechanism, an HCV virocidal assay was performed. Briefly, peptide #1 was diluted in complete growth medium containing 2×10⁵ ffu/mL of HCV to a final concentration of 18 μM. The virus-peptide mixture was incubated for 4 hours at 37° C. The samples were analyzed by three different assays as follows.

In the HCV infectivity assay, the sample was further diluted 250-fold in growth medium to a concentration where the peptide has no inhibitory effect on HCV infection. The residual infectivity was determined by placing the diluted samples on Huh-7.5.1 cells, and cells were stained with antibody against HCV E2 protein 72 hours later. The results (FIG. 11C) indicate that preincubation of virus with peptide 1 completely abolishes viral infectivity.

In the total HCV RNA assay, total RNA of 10 ρL sample was directly isolated by the guanidine thiocyanate method. The HCV RNA transcript level was measured by real time RT-QPCR, and normalized to the level of GAPDH released into viral supernatant during CPE. Results (FIG. 11D) show that preincubation of virus with peptide 1 reduces the total viral RNA content by at least 3-fold, suggesting viral lysis.

Sucrose density gradient was used to examine whether the antiviral effect of peptide 1 on total HCV RNA and HCV infectivity was limited to a subset of HCV particles. In this method, the peptide-treated and control virus samples (250 μL) were resolved on a sucrose density gradient and fractions were analyzed for infectivity and viral RNA content. Gradients were formed by equal volume (700 μL) steps of 20%, 30%, 40%, 50% and 60% sucrose solutions in TNE buffer (10 mM Tris-HCl pH 8,150 mM NaCl, 2 mM EDTA). Equilibrium was reached by ultracentrifugation (SW41Ti rotor, Beckman Instruments, Palo Alto, Calif.) for 16 hours at 120,000 g at 4° C. Fifteen fractions of 250 μL were collected from the top and analyzed for both HCV RNA and virus infectivity titers. The density of each fraction was determined by measuring the mass of 100 μL aliquot in each sample. The results (FIG. 11E) show that preincubation of virus with peptide completely abolishes infectivity in all fractions and reduces the viral RNA content of all fractions by approximately 4-5 fold, further suggesting viral lysis.

### Example 7

### Comparisons of the L and D-Forms of Peptide 1

Peptides composed of L-amino acids are susceptible to proteolysis, which could shorten their half-life and, thus, their biological activity. To examine this possibility and to determine if specific peptide-viral protein interactions mediate antiviral activity, peptide 1 was synthesized using all D-amino acids, purified to >95% homogeneity, and its antiviral activity and serum stability were compared with a similarly pure preparation of the L-type version of peptide #1. Both L- and D-type peptides were diluted 1:100 in complete growth medium (10% FBS) and mixed with an equal volume of viral supernatant.

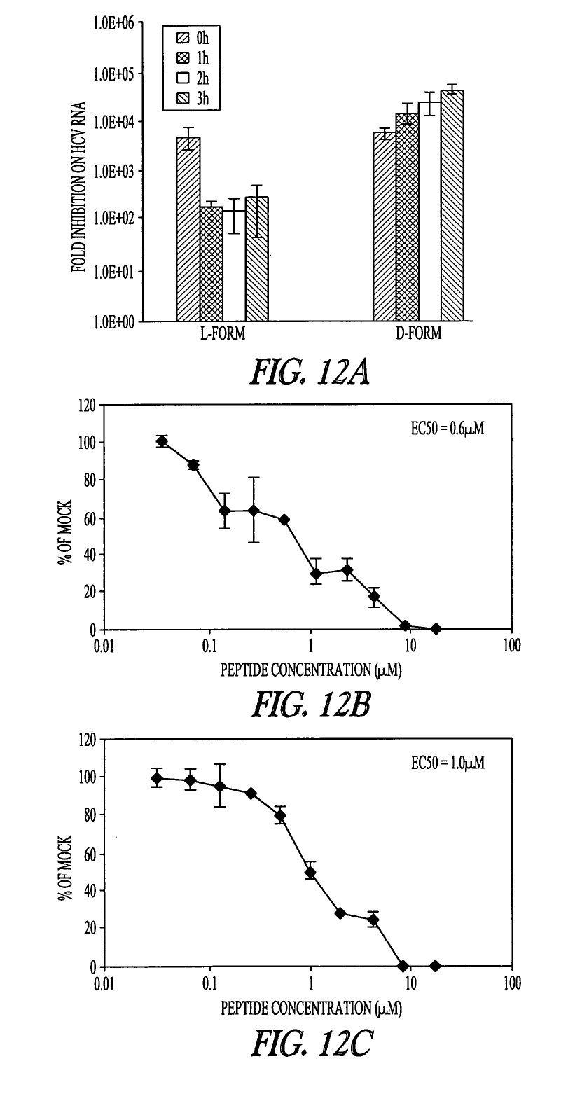

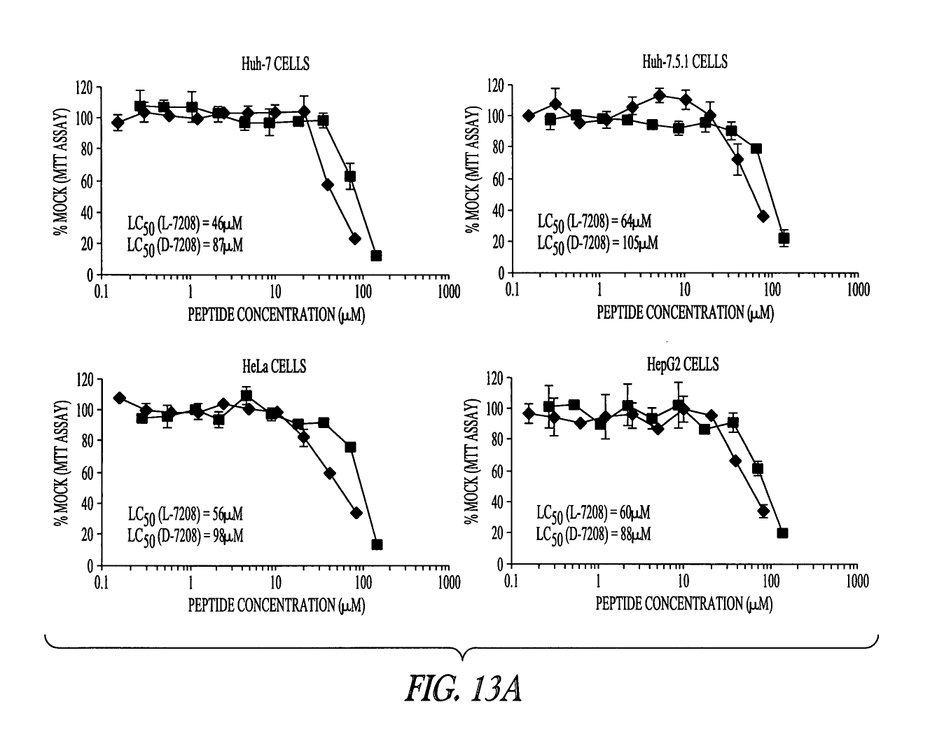

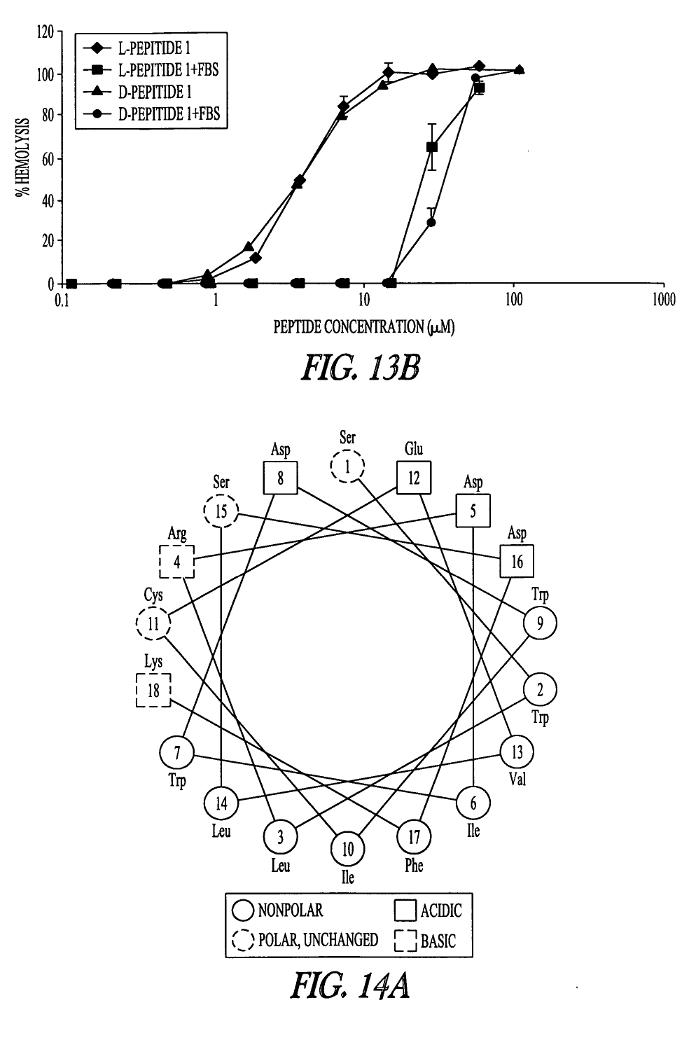

In addition, to compare the serum stability of the L- and D-type peptides, the diluted peptide was incubated at 37° C. for 1 hour, 2 hours and 4 hours before mixing with viral supernatant. The virus-peptide mixture was then used to infect Huh-7.5.1 cells (MOI=0.1). After adsorption for 4 hours at 37° C., the virus-peptide inoculum was removed. The cells were washed 2 times, overlaid with 120 μL fresh growth medium and incubated at 37° C. for 3 days. Cells were lysed and subjected to RNA analysis. The HCV RNA transcript level was measured by real time RT-QPCR and normalized to cellular GAPDH levels. The results (FIG. 12A) show that whereas approximately 95% of the antiviral activity of the L-peptide was lost within 1 hour in 10% FBS at 37° C., the D-peptide was entirely stable for at least 4 hours under the same conditions. Thus, in addition to low immunogenicity and possible oral bioavailability, peptides composed of D-amino acids have the potential therapeutic advantage of enhanced serum stability.

To determine the median effective concentration (EC₅₀) of the L- and D-form of peptide #1, peptide stock solution (3.6 mM in DMSO) was serially diluted 2-fold in DMSO. An aliquot of peptide from each dilution was then diluted 1:100 in complete growth medium and mixed with equal volume of virus supernatant. The virus-peptide mixture was then used to infect Huh-7.5.1 cells (MOI=0.1). After adsorption for 4 hours at 37° C., the virus-peptide inoculum was removed. The cells were washed 2 times, overlaid with 120 μL fresh growth medium and incubated at 37° C. for 3 days. Cells were lysed and subjected to RNA analysis. The HCV RNA transcript level was measured by real time RT-QPCR and normalized to cellular GAPDH levels. The inhibition of HCV infection was calculated by comparing the intracellular HCV RNA transcript between the peptide treatment and solvent control. The results (FIG. 12B-C) indicate that the EC₅₀ values of the L- and D-forms of peptide 1 are virtually identical.

### Example 8

### Peptide Toxicity

Peptide cytotoxicity was measured by MTT cytotoxicity assay based on the protocol provided in the ATCC MTT assay kit (Cat #30-1010K). In brief, 5000-10,000 cells were seeded per well in a 96 well plate. Following overnight growth, 100 μL fresh medium plus 20 μL of 2-fold serially diluted peptide was added. Media without peptides was added to at least 3 wells as untreated controls. The cells were then incubated for 72 hours at 37° C., 5% CO₂. After this incubation, 1/10 volume of MTT solution (5 μg/mL in PBS) was added to each well, and the cells were returned to the incubator. Two hours later, the medium was removed, 150 μL DMSO was added to dissolve the purple precipitate formazan, and the plate was shaken at 150 rpm for 10 minutes. Absorbance at 570 nm less background at 670 nm is a reliable measure of cell death. Cytotoxicity (LD₅₀) of individual peptides was defined as the peptide concentration that caused 50% cell death. The results (FIG. 13A) show that the LD₅₀ values of the L- and D-forms of peptide 1 are virtually identical (3.8 and 3.7 μM, respectively, without FBS; and 26.7 and 36.8 μM with FBS).

Fresh human blood (treated with EDTA) was centrifuged 1000 g for 10 minutes to remove the supernatant and buffy coat. The red blood cells were then washed twice in PBS, and resuspended to a final concentration of 8% with and without 16% FBS. Serial 2-fold dilutions of peptide were prepared in 60 μL PBS in a 96-well microtiter plate, and 60 μL of the suspended human red blood cells with and without FBS were added. The plates were incubated for 1 hour at 37° C. After this incubation 120 μL PBS was added to each well and the plates were centrifuged at 1000 g for 5 mins. Aliquots of 100 μL of supernatant were transferred to a new 96-well microtiter plate. Hemoglobin release is monitored using microplate ELISA reader by measuring the absorbance at 414 nm. In the plate, zero and 100% hemolysis are determined in PBS and 0.1% Triton X-100, respectively. Percent hemolysis as determined according to the formula:
[(A_{414 nm} in the peptide solution−A_{414 nm} in PBS)/(A_{414 nm} in 0.1% Triton X-100−A_{414 nm} in PBS)]×100.

The results (FIG. 13B) indicate that the LC₅₀ values of the L- and D-peptides against human red blood cells, when tested in the presence of serum, were similar to each other and similar to their LC₅₀ against hepatocyte cell lines in vitro. Importantly, the LC₅₀ values against both cell types is consistently 50 to 100-fold higher than the EC₅₀ values for each peptide.

As a preliminary measurement of the in vivo cytotoxicity of the peptides 1, 2 and 3 (see Table 4) a group of three mice (BALB/c mice, 7 weeks old, about 23 g) were each injected with 92 μg L-type peptide 1 ( ˜4 mg/kg) in 200 μL PBS (spun 14,000 rpm for 3 minutes before injection). In the control group, each of three mice was given 200 μL PBS containing 5% DMSO. The mice were monitored for acute toxicity during the first 3 hours after injection. Results are summarized in the following table.

TABLE 6
Peptides 1, 2 and 3 are Nontoxic in C57BL/6 Mice

| | Weight(g) | Weight(g) | Weight(g) | Weight(g) | Weight(g) |
|---|---|---|---|---|---|
| Mice | (d.0) | (d.3) | (d.5) | (d.7) | (d.10) |
| DMSO-1 | 25.3 | 25.3 | 25.6 | 25.7 | 25.5 |
| DMSO-2 | 23.1 | 24.4 | 24.6 | 24.8 | 25.1 |
| DMSO-3 | 22.3 | 22.7 | 23.1 | 23.2 | 23.2 |
| Peptide 1 | 22.2 | 22.3 | 22.8 | 23.1 | 23.5 |
| Peptide 2 | 25.3 | 25.6 | 25.9 | 25.9 | 25.6 |
| Peptide 3 | 24.1 | 24.1 | 24.7 | 24.7 | 24.7 |

No change in appearance, activity or behavior was observed. The mice were then weighed on days 0, 3, 5, 7 and 10. Peptide-injected mice gained weight at the same rate as the controls.

### Example 9

### Physical Properties of Peptide 1 Correlate with its Antiviral Activity

The secondary structure of peptide 1 (SEQ ID NO:43) was analyzed using the tool of helical Wheel Applet available online at cti.itc.virginia.edu/˜cmg/Demo/wheel/wheelApp.html (last visited Aug. 15, 2006). The resulting helical wheel (FIG. 14A) shows that peptide 1 is amphipathic, having both hydrophobic and hydrophilic faces.

The secondary structure of peptide 1 was also analyzed using circular dichroism (CD) spectroscopy using an Aviv model 62DS CD spectrometer (Aviv Associates Inc., Lakewood, N.J.). The CD spectra of peptides were measured at 25° C. using a 1 mm path-length cell. Three scans per sample were performed over the wavelength range of 190 to 260 nm in 10 mM potassium phosphate buffer, pH 7.0. Data were collected at 0.1 nm interval with a scan rate of 60 nm/min and is given in mean molar ellipticity [q]. The peptide concentrations were 50 μM. Spectra highly characteristic of amphotropic α-helices were observed for the L and D form of peptide 1 (FIG. 14B). In addition, dansylation enhances the amphotropic α-helical structure of peptide 1 (FIG. 14C). Thus, the peptides of the invention can have dansyl moieties covalently attached thereto.

The secondary structures of various truncated derivatives of peptide 1 (Table 7) were analyzed using CD spectroscopy. Results indicate that a deletion of 2 or 4 amino acids from the C-terminus of peptide 1 did not eliminate the α-helical structure of the peptide (FIG. 14D). In contrast, deletion of 2 amino acids from the N-terminus of peptide 1 did eliminate the Ε-helical structure of the peptide (FIG. 14E).

The anti-HCV activity of these truncated variants of peptide 1 were also determined. Results (Table 7) indicate that the antiviral activity of peptide 1 (L-form) correlates with its α-helical structure.

TABLE 7

C- and N-terminal Truncation Derivatives
of Peptide 1

| | | | |
|---|---|---|---|
| | | SEQ ID | Anti-HCV |
| Peptide | Sequence | NO: | Activity |

| | | | |
|---|---|---|---|
| Peptide 1 | SWLRDIWDWICEVLSDFK | 43 | + |
| (18mer) | | | |
| \hat{ }C-16mer | SWLRDIWDWICEVLSD | 94 | + |
| \hat{ }C-14mer | SWLRDIWDWICEVL | 92 | + |
| \hat{ }C-13mer | SWLRDIWDWICEV | 103 | − |
| \hat{ }C-12mer | SWLRDIWDWICE | 104 | − |
| \hat{ }C-10mer | SWLRDIWDWI | 105 | − |
| \hat{ }C-8mer | SWLRDIWD | 106 | − |
| \hat{ }N-16mer | LRDIWDWICEVLSDFK | 107 | − |
| \hat{ }N-14mer | DIWDWICEVLSDFK | 108 | − |
| \hat{ }N-12mer | WDWICEVLSDFK | 109 | − |
| \hat{ }N-10mer | WICEVLSDFK | 110 | − |
| \hat{ }N-8mer | CEVLSDFK | 111 | − |

### Example 10

### Liposome-Dye Release Assay

Liposomes (Large Unilamellar Vesicles, LUV) were prepared as follows. Lipid mixture containing 28 mg of total lipids (12 mM) in the proportions composed of 10POPC:11DPPC:1POPS:6Cholestrol (Avanti Polar Lipids, Inc., Alabaster, Ala.) were dissolved in 1 mL chloroform, 1 mL ether, and 2 mL sulforhodamine B (100 mM in 10 mM Hepes, pH 7.2; SulfoB, Molecular Probes). The mixture was sonicated at 4° C. using a Branson 2210 water bath sonicator for 10 minutes. After organic solvents were removed using a vacuum Buchi Rotavapor R-114, the lipids were resuspended in 2 mL of sulforhodamine B. The mixture was vaporated until foaming stops. The lipid vesicles were sized by repeated extrusion 8 times through a stack of 0.8, 0.4, and 0.2 μm polycarbonate membrane filters using a Mini-Extruder (Avanti Polar Lipids, Inc., Alabaster, Ala.). The liposomes loaded with sulforhodamine B were separated from unencapsulated sulforhodamine B on a Sephadex G-25 column.

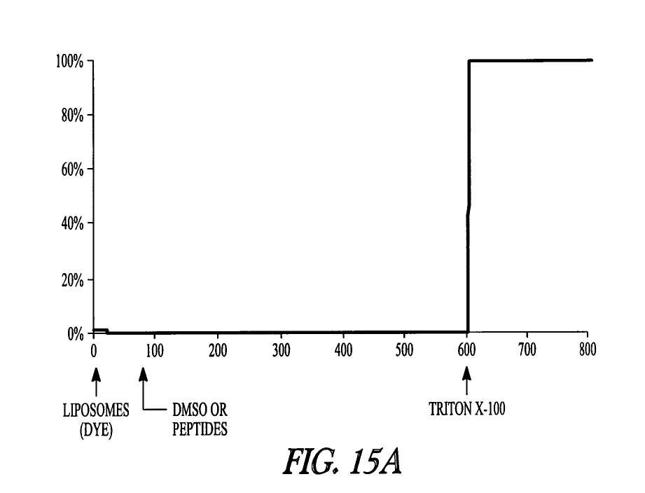

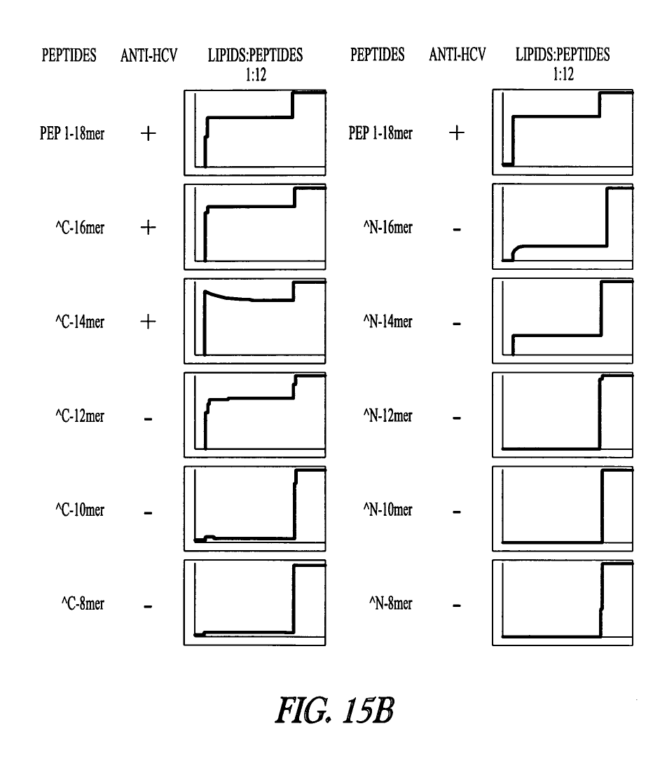

Dye release assays were performed in an Aminco-Bowman Series 2 Luminescence Spectrometer (Thermo Electron Corporation, Waltham, Mass.). Ten microliters of liposomes were diluted to a final concentration of 120 μM in 978 μL Hepes buffer in a stirred cuvette at room temperature. The samples were excited at a wavelength of 535 nm, and emission was monitored at 585 nm. After 60 seconds equilibration, 10 μL of peptides were added to the cuvette and the kinetics of membrane disruption were monitored by the increase in sulforhodamine B fluorescence. The percentage of sulforhodamine B released by the addition of peptides was calculated using the following formula:
% sulforhodamine B released=100×(F−Fₒ)/(F₁₀₀−Fₒ),
where F is the fluorescence intensity achieved by the peptides, Fₒ is the basal fluorescence intensity acquired upon addition of peptide, and F₁₀₀ is the fluorescence intensity corresponding to 100% sulforhodamine B release obtained by the addition of 25 μL of 10% Triton X-100. (FIG. 15A)

The peptides in Table 7 were tested in this assay. Results (FIG. 15B) indicate that the antiviral activity of the various derivatives of peptide 1 correlates with the ability to cause liposome dye release. Thus, the antiviral activity of peptide 1 correlates with the α-helical structure and liposome dye release as summarized in the following table.

TABLE 8
Structure/function Relationship of Peptide 1
and Truncations Thereof

| C-terminal truncations | | | | N-terminal truncations | | | |
|---|---|---|---|---|---|---|---|
| | Anti- | α- | Dye | | Anti- | α- | Dye |
| Peptide | HCV | helix | release | Peptide | HCV | helix | release |
| Peptide 1 | + | + | + | Peptide 1 | + | + | + |
| \hat{ }C- | + | + | + | \hat{ }N-16mer | − | − | − |
| 16mer | | | | | | | |
| \hat{ }C- | + | + | + | \hat{ }N-14mer | − | − | − |
| 14mer | | | | | | | |
| \hat{ }C- | − | − | − | \hat{ }N-12mer | − | − | − |
| 12mer | | | | | | | |
| \hat{ }C- | − | − | − | \hat{ }N-10mer | − | − | − |
| 10mer | | | | | | | |
| \hat{ }C- | − | − | − | \hat{ }N-8mer | − | − | − |
| 8mer | | | | | | | |

### Example 11

### Antiviral Activity and Primary Structure

To determine whether the antiviral activity of peptide 1 is dependent on its primary amino acid sequence, four derivative peptides from peptide 1 were synthesized to a purity >95%. The four derivatives having the same composition of amino acids included (1) the reversed the sequence of peptide 1 (also called retro-peptide); (2) scrambled hydrophobic amino acids; (3) scrambled hydrophilic amino acids; and (4) a derivative in which the aspartic acid residues (D) were replaced with proline residues (P). The antiviral activity of the peptides was examined by HCV focus reduction assay at three peptide concentrations: 18 μM, 6 μM and 2 μM, as described above.

Results, which are summarized in the following table shows that the antiviral activity of peptide 1 correlates with the α-helical structure, but not with the primary amino acid sequence.

TABLE 9

Antiviral Activity of Scrambled Derivatives
of Peptide 1

| | SEQ ID | Final | No | 1/3 | 1/9 |
|---|---|---|---|---|---|
| Peptide Sequences | NO: | Concentration | dilution | diluted | diluted |
| SWLRDIWDWICEVLSDFK | 43 | 18 μM | 0 ± 0 | 31 ± 2 | 44 ± 8 |
| (L-a.a) | | | | | |
| KFDSLVECIWDWIDRLWS | 96 | 18 μM | 1 ± 1 | 50 ± 7 | 31 ± 3 |
| (L-a.a, Retro) | | | | | |
| SWLRDIWDWICEVLSDFK | NA | 17 μM | 0 ± 0 | 8 ± 6 | 10 ± 2 |
| (D-a.a) | | | | | |
| SIWRDWVDLICEFLSDWK | 97 | 19 μM | 0 ± 0 | 31 ± 2 | 16 ± 4 |
| (L-, hydrophobic | | | | | |
| scrambled) | | | | | |
| KWLCRIWSWISDVLDDFE | 98 | 20 μM | 0 ± 0 | 8 ± 6 | 15 ± 6 |
| (L-, hydrophobic | | | | | |
| scrambled) | | | | | |
| SWLRPIWPWICEVLSDFK | 91 | 19 μM | 9 ± 3 | 21 ± 8 | 41 ± 2 |
| (L-, 2D/P) | | | | | |
| MOCK | NA | 0.5% DMSO | 53 ± 4 | | |

In sum, by screening a synthetic HCV peptide library, 13 peptides were identified that could inhibit HCV infection efficiently. Peptide 1, for example, derived from the membrane anchor domain of NS5A (NS5A-1975) was highly potent as a single dose of this peptide completely blocked HCV infection with an EC₅₀ of 289 nM without evidence of cytotoxicity. The antiviral effect was evident for at least 11 days post infection. The peptide was most active when it was added to the cells together with the virus. Preincubation of the peptide with virus significantly reduced viral attachment and infectivity, suggesting that the antiviral activity of NS5A-1975 interacts directly with the virus and destabilizes it. The D-amino acid form of the peptide is fully active, and the D- and L-forms of the peptide display amphipathic α-helical structure in solution and induce permeabilization of artificial liposomes. Importantly, the antiviral activity of a series of N— and C-terminally truncated NS5A-1975 peptides correlated perfectly with their membrane permeability activity and amphipathic α-helical structure. In contrast, NS5A-1975 had no effect on several other enveloped RNA viruses, including vesicular stomatitis virus, lymphocytic choriomeningitis virus and Boma disease virus. Thus, peptide 1 is a potent HCV-derived synthetic α-helical peptide that blocks HCV infection by inactivating the virus extracellularly. These results suggest that NS5A-1975 may represent a novel therapeutic strategy for HCV infection.

### Example 12

### Effect of Peptides on VSV Infection

To determine whether the antiviral activity of peptide #1 is specific for HCV, similar experiments were conducted on other enveloped viruses, e.g. vesicular stomatitis virus (VSV). Two assays were used to test the antiviral activity of peptide #1 against VSV.

Blockade of infection. To examine if peptide 1 blocks VSV infection, peptide 1 at final concentration 18 μM and VSV from 1 to 10,000 pfu/mL were concurrently added to Huh-7 cells. In parallel, peptide and HCV (10,000 ffu/mL) were added to cells as control. After adsorption for 4 hours at 37° C., the virus-peptide inoculum was removed. The cells were washed 2 times, overlaid with 120 μL fresh growth medium and incubated at 37° C. for 3 days. VSV and HCV infections were assessed by viral cytopathic effect (CPE) and immunostaining with antibody against HCV E2 protein, respectively.

Virocidal activity. To determine if peptide 1 has virucidal activity against VSV, peptide 1 was diluted in a complete growth medium containing 2×10⁵ pfu (ffu)/mL VSV or HCV to a final concentration of 18 μM. The virus-peptide mixture was then incubated for 4 hours at 37° C. The VSV and HCV viral titer were then determined by serial dilution and assessed by viral cytopathic effect (CPE) and immunostaining with antibody against HCV E2 protein, respectively.

The result (FIG. 8) indicates that peptide 1 does not block VSV infection and has no virocidal activity against VSV.

### Example 13

### Effect of Peptides on Dengue-2 Infection

The following experiments were performed to determine which peptides inhibited Dengue-2 viral infection.

Enzyme-linked Immunosorbent Assay. Vero cells (80,000 cells/well/ml) were seeded for 24 h pre-infection in 24-well plates. Cells were exposed to Dengue-2 (derived from Vero cells) in the presence of increasing concentration of peptide (or DMSO as control). Viruses and peptide were not removed (cells were not washed) throughout the incubation. Infection was analyzed after 5 days using ELISA that measured the amounts of Dengue-2 capsid released in the supernatant of infected Vero cells.

Fluorescent Foci Assay: Vero cells were seeded for 24 h pre-infection in 96-well plates. Cells were exposed to Dengue-2 in the presence of increasing concentrations of peptide (or DMSO as control). Viruses and peptide were washed away 2 h post-infection. Supernatants were collected every 3 days post-infection and added to fresh Vero cells for fluorescent foci assay. Newly infected Vero cells were fixed with 4% formaldehyde after 3 days. Cells were then stained with Dengue Env antibodies followed by Alexa-fluor dye conjugated secondary antibodies. Foci were counted using a fluorescent microscope.

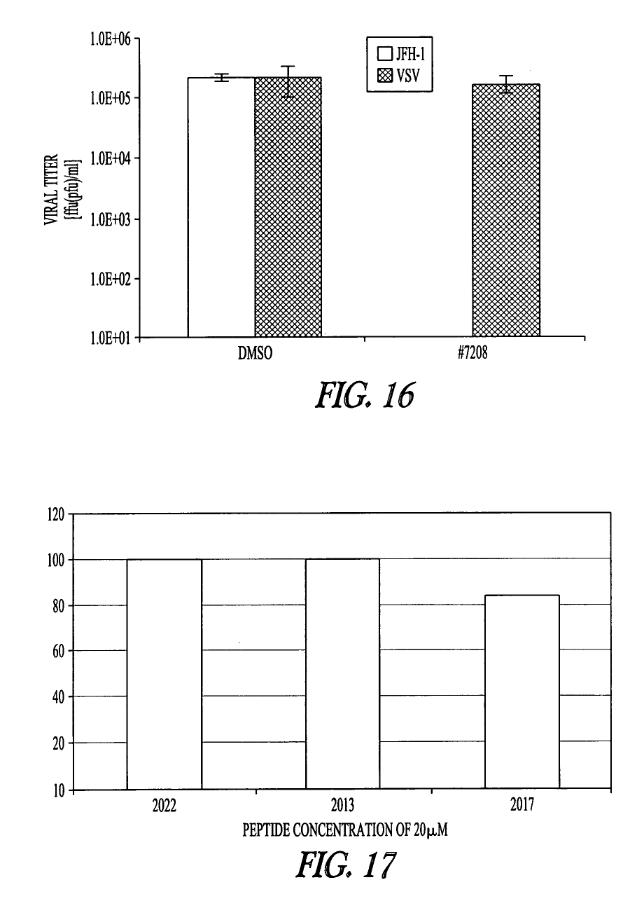

Results are summarized in the following table and in FIG. 17.

TABLE 10

Inhibition of Dengue Infection as Detected
by ELISA

| | | | |
|---|---|---|---|
| | | α-DEN | α-Capsid |
| Peptides | Sequences | ENV | 9A7 |

| | | | |
|---|---|---|---|
| 2022 peptide | SWLRDIWDWICEVLSDFK | 97.8 | 98.02 |
| (20 μM) | (SEQ ID NO:43) | | |
| 2022 peptide | | 28.0 | 50.02 |
| (5 μM) | | | |
| 2022 peptide | | 0 | 0 |
| (1.25 μM) | | | |
| 2013 peptide | SWLRDIWDWICEVL | 97.8 | 98.3 |
| (20 μM) | (SEQ ID NO:92) | | |
| 2013 peptide | | 29.65 | 22.7 |
| (5 μM) | | | |
| 2013 peptide | | 0 | 11.4 |
| (1.25 μM) | | | |
| 2017 peptide | LRDIWDWICEVLSDFK | 74.83 | 82.2 |
| (20 μM) | (SEQ ID NO:107) | | |
| 2017 peptide | | 33.64 | 16.01 |
| (5 μM) | | | |
| 2017 peptide | | 10.24 | 12.82 |
| (1.25 μM) | | | |

As illustrated in Table 10 and FIG. 17, Dengue infection was inhibited by the present peptides in a dose-dependent manner. Essentially 100% inhibition of Dengue viral infection was observed at concentrations of 20 μM (FIG. 17).

Intracellular FACS Assay: Vero cells were seeded for 24 h pre-infection in 6-well plates. Cells were exposed to Dengue-2 in the presence of increasing concentrations of peptide (or DMSO as control). Viruses and peptide were washed away 2 h post-infection. Cells were taken for intracellular staining 3 days post-infection. Cells were stained with appropriate isotype control, Dengue Env, Dengue capsid or tubulin antibodies. Cells were analyzed by FACS.

Results when using peptide concentrations of 20 μM are shown in Table 11. Results for 1.25 to 20 μM are summarized in the graph shown in FIG. 18.

TABLE 11
Inhibition of Dengue Infection as Detected by FACS

% GATED from Intracellular
FACS Staining of Cells
3 Days Post-infection

| | | | | | |
|---|---|---|---|---|---|
| | | | Dengue | | % of Dead |
| | SEQ ID | Isotype | α-Capsid | α- | cells |
| Sequence | NO: | Control | (9A7) | Tubulin | PI* |

| | | | | | | |
|---|---|---|---|---|---|---|
| No Virus | N/A | N/A | 1.29 | 0.08 | 91.96 | 1.00 |
| Control | | | | | | |
| DMSO | N/A | N/A | 0.39 | 41.26 | 96.67 | 1.00 |
| 2015 | SWLRDIWDWI | 105 | 0.85 | 37.21 | 97.30 | 1.2 |
| peptide | | | | | | |
| 2054 | SWLRDIWDWICEV | 103 | 1.68 | 2.61 | 97.35 | 3.04 |
| peptide | | | | | | |
| 2018 | DIWDWICEVLSDFK | 108 | 0.97 | 12.07 | 96.83 | 0.76 |
| peptide | | | | | | |
| L-2022 | SWLRDIWDWICEVLSDFK | 43 | 0.68 | 0.82 | 95.65 | 1.88 |
| peptide | | | | | | |
| D-2022 | SWLRDIWDWICEVLSDFK | N/A | 0.77 | 1.35 | 92.60 | 3.83 |
| peptide | | | | | | |
| 6938 | LYGNEGCGWAGWLLSPRG | 6 | 0.69 | 42.41 | 96.32 | 4.05 |
| peptide | | | | | | |

*PI-Propidium iodide staining for measuring dead cells; N/A - non-applicable

As shown in Table 11 and FIG. 18, the present peptides inhibit Dengue viral infection in a dose-dependent manner. Essentially 100% inhibition of Dengue viral infection was observed at concentrations of 20 μM (FIG. 18).

Fluorescent Foci Assay. Vero cells were seeded for 24 hours pre-infection in 96-well plates. Cells were exposed to Dengue-2 in the presence of increasing concentrations of peptide (or DMSO as control). Viruses and peptide were washed away 2 hours post-infection. Supernatants were collected every 3 days post-infection and added to fresh Vero cells for fluorescent foci assay. Newly infected Vero cells were fixed with 4% formaldehyde after 3 days. Cells were then stained with antibodies directed to the Dengue Envelop protein followed by Alexa-fluor dye conjugated secondary antibodies. Foci were counted using a fluorescent microscope.

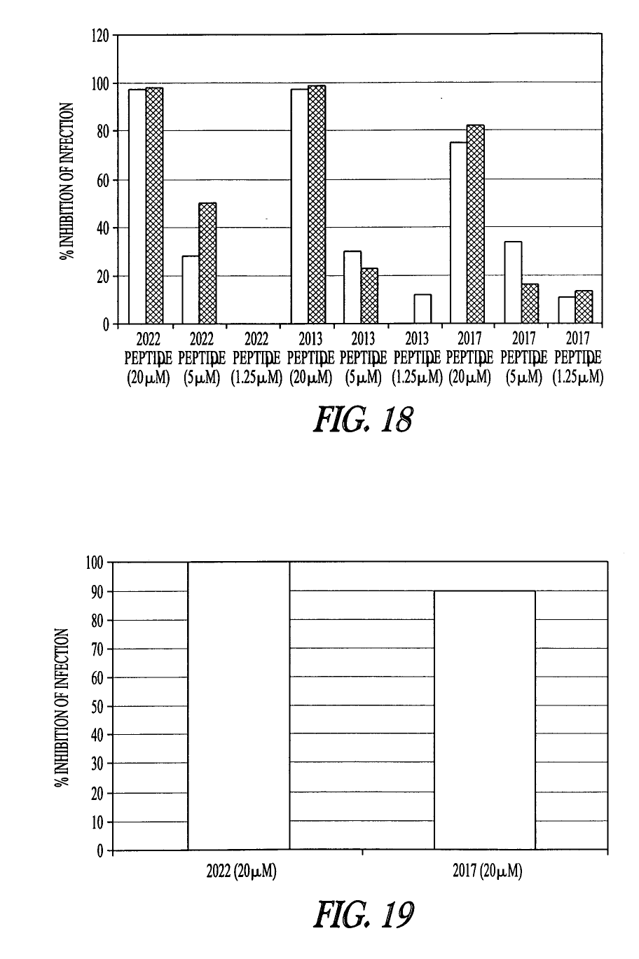

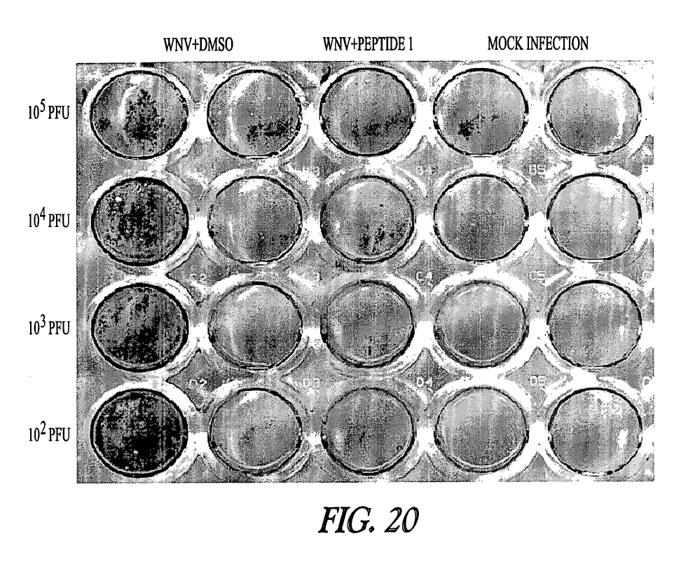

The results shown in FIG. 19 further confirm that the present peptides strongly inhibit Dengue viral infection. Essentially 100% inhibition of Dengue viral infection was observed at concentrations of 20 μM (FIG. 19).

### Example 14

### Peptide 1 has Strong Antiviral Activity Against West Nile Viral Infection

In this study, the activity of peptide 1 against the West Nile Virus (WNV), a Flavivirus, was examined. A549 cells were infected with 10² to 10⁵ PFU/mL WNV (New York strain) in the presence of 0.5% DMSO or peptide 1 (final concentration 18 μM in 0.5% DMSO). After 3 days of incubation at 37° C., the cells were fixed and subjected to immuno-peroxidase staining to detect WNV protein. Results (FIG. 20) show that the cell monolayer with 10⁵ PFU/mL treated with DMSO was almost completely destroyed, and all the cells in the lower titer wells expressed WNV protein. In contrast, the monolayers in the peptide-treated cells were intact, and little or no WNV protein was detected. In particular, the WNV protein staining intensity was the same as the uninfected negative control wells, irrespective of the dose of the viral inoculum. These results demonstrate that peptide 1 (SEQ ID NO:43) has a strong antiviral activity against WNV infection.

### DOCUMENTS

1. Hoofnagle, J. H. (2002) Hepatology 36, S21-29.
2. Kanto, T., Hayashi, N., Takehara, T., Tatsumi, T., Kuzushita, N., Ito, A., Sasaki, Y., Kasahara, A. & Hori, M. (1999) J. Immunol. 162, 5584-5591.
3. Auffermann-Gretzinger, S., Keeffe, E. B. & Levy, S. (2001) Blood 97, 3171-3176.
4. Hiasa, Y., Horiike, N., Akbar, S. M., Saito, I., Miyamura, T., Matsuura, Y. & Onji, M. (1998) Biochem. Biophys. Res. Commun. 249, 90-95.
5. Alter, H. J. & Seeff, L. B. (2000) Semin. Liver Dis. 20, 17-35.
6. Thimme, R., Oldach, D., Chang, K. M., Steiger, C., Ray, S. C. & Chisari, F. V. (2001) J. Exp. Med. 194, 1395-1406.
7. Takaki, A., Wiese, M., Maertens, G., Depla, E., Seifert, U., Liebetrau, A., Miller, J. L., Manns, M. P. & Rehermann, B. (2000) Nat. Med. 6, 578-582.
8. Lechner, F., Wong, D. K., Dunbar, P. R., Chapman, R., Chung, R. T., Dohrenwend, P., Robbins, G., Phillips, R., Klenerman, P. & Walker, B. D. (2000) J. Exp. Med. 191, 1499-1512.
9. Logvinoff, C., Major, M. E., Oldach, D., Heyward, S., Talal, A., Balfe, P., Feinstone, S. M., Alter, H., Rice, C. M. & McKeating, J. A. (2004) Proc. Natl. Acad. Sci. U.S.A. 101, 10149-10154.
10. Shoukry, N. H., Sidney, J., Sette, A. & Walker, C. M. (2004) J. Immunol. 172, 483-492.
11. Thimme, R., Bukh, J., Spangenberg, H. C., Wieland, S., Pemberton, J., Steiger, C., Govindarajan, S., Purcell, R. H. & Chisari, F. V. (2002) Proc. Natl. Acad. Sci. U.S.A. 99, 15661-15668.
12. Bukh, J. (2004) Hepatology 39, 1469-1475.
13. Ikeda, M., Yi, M., Li, K. & Lemon, S. M. (2002) J. Virol. 76, 2997-3006.
14. Lohmann, V., Komer, F., Koch, J., Herian, U., Theilmann, L. & Bartenschlager, R. (1999) Science 285, 110-113.
15. Gale, M., Jr. (2003) Hepatology 37, 975-978.
16. Katze, M. G., He, Y. & Gale, M., Jr. (2002) Nat Rev Immunol 2, 675-687.
17. Dubuisson, J. & Rice, C. M. (1996) J. Virol. 70, 778-786.
18. Ye, J., Wang, C., Sumpter, R., Jr., Brown, M. S., Goldstein, J. L. & Gale, M., Jr. (2003) Proc. Natl. Acad. Sci. U.S.A. 100, 15865-15870.
19. Kapadia, S. B. & Chisari, F. V. (2005) Proc. Natl. Acad. Sci. U.S.A. 102, 2561-2566.
20. Lohmann, V., Komer, F., Dobierzewska, A. & Bartenschlager, R. (2001) J. Virol. 75, 1437-1449.
21. Blight, K. J., Kolykhalov, A. A. & Rice, C. M. (2000) Science 290, 1972-1975.
22. Kato, T., Date, T., Miyamoto, M., Furusaka, A., Tokushige, K., Mizokami, M. & Wakita, T. (2003) Gastroenterology 125, 1808-1817.
23. Kato, T., Furusaka, A., Miyamoto, M., Date, T., Yasui, K., Hiramoto, J., Nagayama, K., Tanaka, T. & Wakita, T. (2001) J. Med. Virol. 64, 334-339.
24. Date, T., Kato, T., Miyamoto, M., Zhao, Z., Yasui, K., Mizokami, M. & Wakita, T. (2004) J. Biol. Chem. 279, 22371-22376.
25. Kato, T., Date, T., Miyamoto, M., Zhao, Z., Mizokami, M. & Wakita, T. (2005) J. Virol. 79, 592-596.
26. Wakita, T., Kato, T., Date, T. & Miyamoto, M. (2004) 11th International Symposium on Hepatitis C & related Viruses, Heidelberg, Germany.
27. Wakita, T., Pietschmann, T., Kato, T., Date, T., Miyamoto, M., Zhao, Z., Murthy, K., Habermann, A., Krausslich, H. G., Mizokami, M., Bartenschlager, R. & Liang, T. J. (2005) Nat. Med. in press.
28. Graham, F. L., Smiley, J., Russell, W. C. & Naim, R. (1977) J. Gen. Virol. 36, 59-74.
29. Gey, G. O., Coffinan, W. D. & Kubicek, M. T. (1952) Cancer Res. 12, 264-265.
30. Knowles, B. B., Howe, C. C. & Aden, D. P. (1980) Science 209, 497-499.
31. Moradpour, D., Evans, M. J., Gosert, R., Yuan, Z., Blum, H. E., Goff, S. P., Lindenbach, B. D. & Rice, C. M. (2004) J. Virol. 78, 7400-7409.
32. Krieger, N., Lohmann, V. & Bartenschlager, R. (2001) J. Virol. 75, 4614-4624.
33. Chomczynski, P. & Sacchi, N. (1987) Anal. Biochem. 162, 156-159.
34. Kapadia, S. B., Brideau-Andersen, A. & Chisari, F. V. (2003) Proc. Natl. Acad. Sci. U.S.A. 100, 2014-2018.
35. Heller, T., Saito, S., Auerbach, J., Williams, T., Moreen, T. R., Jazwinski, A., Cruz, B., Jeurkar, N., Sapp, R., Luo, G. & Liang, T. J. (2005) Proc. Natl. Acad. Sci. U.S.A. 102, 2579-2583.
36. Levy, S., Todd, S. C. & Maecker, H. T. (1998) Annu. Rev. Immunol. 16, 89-109.
37. Blight, K. J., McKeating, J. A. & Rice, C. M. (2002) J. Virol. 76, 13001-13014.
38. Bartosch, B., Dubuisson, J. & Cosset, F. L. (2003) J. Exp. Med. 197, 633-642.
39. Hsu, M., Zhang, J., Flint, M., Logvinoff, C., Cheng-Mayer, C., Rice, C. M. & McKeating, J. A. (2003) Proc. Natl. Acad. Sci. U.S.A. 100, 7271-7276.
40. Zhang, J., Randall, G., Higginbottom, A., Monk, P., Rice, C. M. & McKeating, J. A. (2004) J. Virol. 78, 1448-1455.
41. Hijikata, M., Shimizu, Y. K., Kato, H., Iwamoto, A., Shih, J. W., Alter, H. J., Purcell, R. H. & Yoshikura, H. (1993) J. Virol. 67, 1953-1958.
42. Trestard, A., Bacq, Y., Buzelay, L., Dubois, F., Barin, F., Goudeau, A. & Roingeard, P. (1998) Arch. Virol. 143, 2241-2245.
43. Bartenschlager, R., Kaul, A. & Sparacio, S. (2003) Antiviral Res. 60, 91-102.
44. Bukh, J., Pietschmann, T., Lohmann, V., Krieger, N., Faulk, K., Engle, R. E., Govindarajan, S., Shapiro, M., St Claire, M. & Bartenschlager, R. (2002) Proc. Natl. Acad. Sci. U.S.A. 99, 14416-14421.
45. Neumann, A. U., Lam, N. P., Dahari, H., Gretch, D. R., Wiley, T. E., Layden, T. J. & Perelson, A. S. (1998) Science 282, 103-107.
46. Tanaka, J., Katayama, K., Kumagai, J., Komiya, Y., Yugi, H., Kishimoto, S., Mizui, M., Tomoguri, T., Miyakawa, Y. & Yoshizawa, H. (2005) Intervirology 48, 120-123.
47. Cormier, E. G., Tsamis, F., Kajumo, F., Durso, R. J., Gardner, J. P. & Dragic, T. (2004) Proc. Natl. Acad. Sci. US.A. 101, 7270-7274.
48. Sumpter, R., Jr., Loo, Y. M., Foy, E., Li, K., Yoneyama, M., Fujita, T., Lemon, S. M. & Gale, M., Jr. (2005) J. Virol. 79, 2689-2699.
49. Yoneyama, M., Kikuchi, M., Natsukawa, T., Shinobu, N., Imaizumi, T., Miyagishi, M., Taira, K., Akira, S. & Fujita, T. (2004) Nat Immunol 5, 730-737.
50. Mbow, M. L. & Sarisky, R. T. (2004) Trends Biotechnol. 22, 395-399.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

```
# SEQUENCE LIS
#TING
<160> NUMBER OF SEQ ID NOS: 118
<210> SEQ ID NO 1
<211> LENGTH: 3011
<212> TYPE: PRT
<213> ORGANISM: Hepatitis C Virus
<400> SEQUENCE: 1
Met Ser Thr Asn Pro Lys Pro Gln Arg Lys Th
#r Lys Arg Asn Thr Asn
  1 5
# 10
# 15
Arg Arg Pro Gln Asp Val Lys Phe Pro Gly Gl
#y Gly Gln Ile Val Gly
                        20
# 25
# 30
Gly Val Tyr Leu Leu Pro Arg Arg Gly Pro Ar
#g Leu Gly Val Arg Ala
                35
# 40
# 45
Thr Arg Lys Thr Ser Glu Arg Ser Gln Pro Ar
#g Gly Arg Arg Gln Pro
        50
# 55
# 60
Ile Pro Lys Ala Arg Arg Pro Glu Gly Arg Th
#r Trp Ala Gln Pro Gly
65
#70
#75
#80
Tyr Pro Trp Pro Leu Tyr Gly Asn Glu Gly Cy
#s Gly Trp Ala Gly Trp
                                85
# 90
# 95
Leu Leu Ser Pro Arg Gly Ser Arg Pro Ser Tr
#p Gly Pro Thr Asp Pro
                        100
# 105
# 110
Arg Arg Arg Ser Arg Asn Leu Gly Lys Val Il
#e Asp Thr Leu Thr Cys
                115
# 120
# 125
Gly Phe Ala Asp Leu Met Gly Tyr Ile Pro Le
#u Val Gly Ala Pro Leu
        130
# 135
# 140
Gly Gly Ala Ala Arg Ala Leu Ala His Gly Va
#l Arg Val Leu Glu Asp
145 1
#50 1
#55 1
#60
Gly Val Asn Tyr Ala Thr Gly Asn Leu Pro Gl
#y Cys Ser Phe Ser Ile
                                165
# 170
# 175
Phe Leu Leu Ala Leu Leu Ser Cys Leu Thr Va
#l Pro Ala Ser Ala Tyr
                        180
# 185
# 190
Gln Val Arg Asn Ser Ser Gly Leu Tyr His Va
#l Thr Asn Asp Cys Pro
                195
# 200
# 205
Asn Ser Ser Ile Val Tyr Glu Ala Ala Asp Al
#a Ile Leu His Thr Pro
        210
# 215
# 220
Gly Cys Val Pro Cys Val Arg Glu Gly Asn Al
#a Ser Arg Cys Trp Val
225 2
#30 2
#35 2
#40
Ala Val Thr Pro Thr Val Ala Thr Arg Asp Gl
#y Lys Leu Pro Thr Thr
                                245
# 250
# 255
Gln Leu Arg Arg His Ile Asp Leu Leu Val Gl
#y Ser Ala Thr Leu Cys
                        260
# 265
# 270
Ser Ala Leu Tyr Val Gly Asp Leu Cys Gly Se
#r Val Phe Leu Val Gly
                275
# 280
# 285
Gln Leu Phe Thr Phe Ser Pro Arg Arg His Tr
#p Thr Thr Gln Asp Cys
        290
# 295
# 300
Asn Cys Ser Ile Tyr Pro Gly His Ile Thr Gl
#y His Arg Met Ala Trp
305 3
#10 3
#15 3
#20
Asp Met Met Met Asn Trp Ser Pro Thr Ala Al
#a Leu Val Val Ala Gln
                                325
# 330
# 335
Leu Leu Arg Ile Pro Gln Ala Ile Met Asp Me
#t Ile Ala Gly Ala His
                        340
# 345
# 350
Trp Gly Val Leu Ala Gly Ile Ala Tyr Phe Se
#r Met Val Gly Asn Trp
                355
# 360
# 365
Ala Lys Val Leu Val Val Leu Leu Leu Phe Al
#a Gly Val Asp Ala Glu
        370
# 375
# 380
Thr His Val Thr Gly Gly Ser Ala Gly Arg Th
#r Thr Ala Gly Leu Val
385 3
#90 3
#95 4
#00
Gly Leu Leu Thr Pro Gly Ala Lys Gln Asn Il
#e Gln Leu Ile Asn Thr
                                405
# 410
# 415
Asn Gly Ser Trp His Ile Asn Ser Thr Ala Le
#u Asn Cys Asn Glu Ser
                        420
# 425
# 430
Leu Asn Thr Gly Trp Leu Ala Gly Leu Phe Ty
#r Gln His Lys Phe Asn
                435
# 440
# 445
Ser Ser Gly Cys Pro Glu Arg Leu Ala Ser Cy
#s Arg Arg Leu Thr Asp
        450
# 455
# 460
Phe Ala Gln Gly Trp Gly Pro Ile Ser Tyr Al
#a Asn Gly Ser Gly Leu
465 4
#70 4
#75 4
#80
Asp Glu Arg Pro Tyr Cys Trp His Tyr Pro Pr
#o Arg Pro Cys Gly Ile
                                485
# 490
# 495
Val Pro Ala Lys Ser Val Cys Gly Pro Val Ty
#r Cys Phe Thr Pro Ser
                        500
# 505
# 510
Pro Val Val Val Gly Thr Thr Asp Arg Ser Gl
#y Ala Pro Thr Tyr Ser
                515
# 520
# 525
Trp Gly Ala Asn Asp Thr Asp Val Phe Val Le
#u Asn Asn Thr Arg Pro
        530
# 535
# 540
Pro Leu Gly Asn Trp Phe Gly Cys Thr Trp Me
#t Asn Ser Thr Gly Phe
545 5
#50 5
#55 5
#60
Thr Lys Val Cys Gly Ala Pro Pro Cys Val Il
#e Gly Gly Val Gly Asn
                                565
# 570
# 575
Asn Thr Leu Leu Cys Pro Thr Asp Cys Phe Ar
#g Lys His Pro Glu Ala
                        580
# 585
# 590
Thr Tyr Ser Arg Cys Gly Ser Gly Pro Trp Il
#e Thr Pro Arg Cys Met
                595
# 600
# 605
Val Asp Tyr Pro Tyr Arg Leu Trp His Tyr Pr
#o Cys Thr Ile Asn Tyr
        610
# 615
# 620
Thr Ile Phe Lys Val Arg Met Tyr Val Gly Gl
#y Val Glu His Arg Leu
625 6
#30 6
#35 6
#40
Glu Ala Ala Cys Asn Trp Thr Arg Gly Glu Ar
#g Cys Asp Leu Glu Asp
                                645
# 650
# 655
Arg Asp Arg Ser Glu Leu Ser Pro Leu Leu Le
#u Ser Thr Thr Gln Trp
                        660
# 665
# 670
Gln Val Leu Pro Cys Ser Phe Thr Thr Leu Pr
#o Ala Leu Ser Thr Gly
                675
# 680
# 685
Leu Ile His Leu His Gln Asn Ile Val Asp Va
#l Gln Tyr Leu Tyr Gly
        690
# 695
# 700
Val Gly Ser Ser Ile Ala Ser Trp Ala Ile Ly
#s Trp Glu Tyr Val Val
705 7
#10 7
#15 7
#20
Leu Leu Phe Leu Leu Leu Ala Asp Ala Arg Va
#l Cys Ser Cys Leu Trp
                                725
# 730
# 735
Met Met Leu Leu Ile Ser Gln Ala Glu Ala Al
#a Leu Glu Asn Leu Val
                        740
# 745
# 750
Ile Leu Asn Ala Ala Ser Leu Ala Gly Thr Hi
#s Gly Leu Val Ser Phe
                755
# 760
# 765
Leu Val Phe Phe Cys Phe Ala Trp Tyr Leu Ly
#s Gly Arg Trp Val Pro
        770
# 775
# 780
Gly Ala Val Tyr Ala Phe Tyr Gly Met Trp Pr
#o Leu Leu Leu Leu Leu
785 7
#90 7
#95 8
#00
Leu Ala Leu Pro Gln Arg Ala Tyr Ala Leu As
#p Thr Glu Val Ala Ala
                                805
# 810
# 815
Ser Cys Gly Gly Val Val Leu Val Gly Leu Me
#t Ala Leu Thr Leu Ser
                        820
# 825
# 830
Pro Tyr Tyr Lys Arg Tyr Ile Ser Trp Cys Me
#t Trp Trp Leu Gln Tyr
                835
# 840
# 845
Phe Leu Thr Arg Val Glu Ala Gln Leu His Va
#l Trp Val Pro Pro Leu
        850
# 855
# 860
Asn Val Arg Gly Gly Arg Asp Ala Val Ile Le
#u Leu Met Cys Val Val
865 8
#70 8
#75 8
#80
His Pro Thr Leu Val Phe Asp Ile Thr Lys Le
#u Leu Leu Ala Ile Phe
                                885
# 890
# 895
Gly Pro Leu Trp Ile Leu Gln Ala Ser Leu Le
#u Lys Val Pro Tyr Phe
                        900
# 905
# 910
Val Arg Val Gln Gly Leu Leu Arg Ile Cys Al
#a Leu Ala Arg Lys Ile
                915
# 920
# 925
Ala Gly Gly His Tyr Val Gln Met Ala Ile Il
#e Lys Leu Gly Ala Leu
        930
# 935
# 940
Thr Gly Thr Tyr Val Tyr Asn His Leu Thr Pr
#o Leu Arg Asp Trp Ala
945 9
#50 9
#55 9
#60
His Asn Gly Leu Arg Asp Leu Ala Val Ala Va
#l Glu Pro Val Val Phe
                                965
# 970
# 975
Ser Arg Met Glu Thr Lys Leu Ile Thr Trp Gl
#y Ala Asp Thr Ala Ala
                        980
# 985
# 990
Cys Gly Asp Ile Ile Asn Gly Leu Pro Val Se
#r Ala Arg Arg Gly Gln
                995
# 1000
# 1005
Glu Ile Leu Leu Gly Pro Ala Asp Gly Met Va
#l Ser Lys Gly Trp Arg
        1010
# 1015
# 1020
Leu Leu Ala Pro Ile Thr Ala Tyr Ala Gln Gl
#n Thr Arg Gly Leu Leu
1025 1030
# 1035
# 1040
Gly Cys Ile Ile Thr Ser Leu Thr Gly Arg As
#p Lys Asn Gln Val Glu
                                1045
# 1050
# 1055
Gly Glu Val Gln Ile Val Ser Thr Ala Thr Gl
#n Thr Phe Leu Ala Thr
                        1060
# 1065
# 1070
Cys Ile Asn Gly Val Cys Trp Thr Val Tyr Hi
#s Gly Ala Gly Thr Arg
                1075
# 1080
# 1085
Thr Ile Ala Ser Pro Lys Gly Pro Val Ile Gl
#n Met Tyr Thr Asn Val
        1090
# 1095
# 1100
Asp Gln Asp Leu Val Gly Trp Pro Ala Pro Gl
#n Gly Ser Arg Ser Leu
1105 1110
# 1115
# 1120
Thr Pro Cys Thr Cys Gly Ser Ser Asp Leu Ty
#r Leu Val Thr Arg His
                                1125
# 1130
# 1135
Ala Asp Val Ile Pro Val Arg Arg Arg Gly As
#p Ser Arg Gly Ser Leu
                        1140
# 1145
# 1150
Leu Ser Pro Arg Pro Ile Ser Tyr Leu Lys Gl
#y Ser Ser Gly Gly Pro
                1155
# 1160
# 1165
Leu Leu Cys Pro Ala Gly His Ala Val Gly Le
#u Phe Arg Ala Ala Val
        1170
# 1175
# 1180
Cys Thr Arg Gly Val Ala Lys Ala Val Asp Ph
#e Ile Pro Val Glu Asn
1185 1190
# 1195
# 1200
Leu Glu Thr Thr Met Arg Ser Pro Val Phe Th
#r Asp Asn Ser Ser Pro
                                1205
# 1210
# 1215
Pro Ala Val Pro Gln Ser Phe Gln Val Ala Hi
#s Leu His Ala Pro Thr
                        1220
# 1225
# 1230
Gly Ser Gly Lys Ser Thr Lys Val Pro Ala Al
#a Tyr Ala Ala Gln Gly
                1235
# 1240
# 1245
Tyr Lys Val Leu Val Leu Asn Pro Ser Val Al
#a Ala Thr Leu Gly Phe
        1250
# 1255
# 1260
Gly Ala Tyr Met Ser Lys Ala His Gly Val As
#p Pro Asn Ile Arg Thr
1265 1270
# 1275
# 1280
Gly Val Arg Thr Ile Thr Thr Gly Ser Pro Il
#e Thr Tyr Ser Thr Tyr
                                1285
# 1290
# 1295
Gly Lys Phe Leu Ala Asp Gly Gly Cys Ser Gl
#y Gly Ala Tyr Asp Ile
                        1300
# 1305
# 1310
Ile Ile Cys Asp Glu Cys His Ser Thr Asp Al
#a Thr Ser Ile Leu Gly
                1315
# 1320
# 1325
Ile Gly Thr Val Leu Asp Gln Ala Glu Thr Al
#a Gly Ala Arg Leu Val
        1330
# 1335
# 1340
Val Leu Ala Thr Ala Thr Pro Pro Gly Ser Va
#l Thr Val Ser His Pro
1345 1350
# 1355
# 1360
Asn Ile Glu Glu Val Ala Leu Ser Thr Thr Gl
#y Glu Ile Pro Phe Tyr
                                1365
# 1370
# 1375
Gly Lys Ala Ile Pro Leu Glu Val Ile Lys Gl
#y Gly Arg His Leu Ile
                        1380
# 1385
# 1390
Phe Cys His Ser Lys Lys Lys Cys Asp Glu Le
#u Ala Ala Lys Leu Val
                1395
# 1400
# 1405
Ala Leu Gly Ile Asn Ala Val Ala Tyr Tyr Ar
#g Gly Leu Asp Val Ser
        1410
# 1415
# 1420
Val Ile Pro Thr Ser Gly Asp Val Val Val Va
#l Ser Thr Asp Ala Leu
1425 1430
# 1435
# 1440
Met Thr Gly Phe Thr Gly Asp Phe Asp Ser Va
#l Ile Asp Cys Asn Thr
                                1445
# 1450
# 1455
Cys Val Thr Gln Thr Val Asp Phe Ser Leu As
#p Pro Thr Phe Thr Ile
                        1460
# 1465
# 1470
Glu Thr Thr Thr Leu Pro Gln Asp Ala Val Se
#r Arg Thr Gln Arg Arg
                1475
# 1480
# 1485
Gly Arg Thr Gly Arg Gly Lys Pro Gly Ile Ty
#r Arg Phe Val Ala Pro
        1490
# 1495
# 1500
Gly Glu Arg Pro Ser Gly Met Phe Asp Ser Se
#r Val Leu Cys Glu Cys
1505 1510
# 1515
# 1520
Tyr Asp Ala Gly Cys Ala Trp Tyr Glu Leu Th
#r Pro Ala Glu Thr Thr
                                1525
# 1530
# 1535
Val Arg Leu Arg Ala Tyr Met Asn Thr Pro Gl
#y Leu Pro Val Cys Gln
                        1540
# 1545
# 1550
Asp His Leu Glu Phe Trp Glu Gly Val Phe Th
#r Gly Leu Thr His Ile
                1555
# 1560
# 1565
Asp Ala His Phe Leu Ser Gln Thr Lys Gln Se
#r Gly Glu Asn Phe Pro
        1570
# 1575
# 1580
Tyr Leu Val Ala Tyr Gln Ala Thr Val Cys Al
#a Arg Ala Gln Ala Pro
1585 1590
# 1595
# 1600
Pro Pro Ser Trp Asp Gln Met Trp Lys Cys Le
#u Ile Arg Leu Lys Pro
                                1605
# 1610
# 1615
Thr Leu His Gly Pro Thr Pro Leu Leu Tyr Ar
#g Leu Gly Ala Val Gln
                        1620
# 1625
# 1630
Asn Glu Val Thr Leu Thr His Pro Ile Thr Ly
#s Tyr Ile Met Thr Cys
                1635
# 1640
# 1645
Met Ser Ala Asp Leu Glu Val Val Thr Ser Th
#r Trp Val Leu Val Gly
        1650
# 1655
# 1660
Gly Val Leu Ala Ala Leu Ala Ala Tyr Cys Le
#u Ser Thr Gly Cys Val
1665 1670
# 1675
# 1680
Val Ile Val Gly Arg Ile Val Leu Ser Gly Ly
#s Pro Ala Ile Ile Pro
                                1685
# 1690
# 1695
Asp Arg Glu Val Leu Tyr Gln Glu Phe Asp Gl
#u Met Glu Glu Cys Ser
                        1700
# 1705
# 1710
Gln His Leu Pro Tyr Ile Glu Gln Gly Met Me
#t Leu Ala Glu Gln Phe
                1715
# 1720
# 1725
Lys Gln Lys Ala Leu Gly Leu Leu Gln Thr Al
#a Ser Arg Gln Ala Glu
        1730
# 1735
# 1740
Val Ile Thr Pro Ala Val Gln Thr Asn Trp Gl
#n Lys Leu Glu Val Phe
1745 1750
# 1755
# 1760
Trp Ala Lys His Met Trp Asn Phe Ile Ser Gl
#y Ile Gln Tyr Leu Ala
                                1765
# 1770
# 1775
Gly Leu Ser Thr Leu Pro Gly Asn Pro Ala Il
#e Ala Ser Leu Met Ala
                        1780
# 1785
# 1790
Phe Thr Ala Ala Val Thr Ser Pro Leu Thr Th
#r Gly Gln Thr Leu Leu
                1795
# 1800
# 1805
Phe Asn Ile Leu Gly Gly Trp Val Ala Ala Gl
#n Leu Ala Ala Pro Gly
        1810
# 1815
# 1820
Ala Ala Thr Ala Phe Val Gly Ala Gly Leu Al
#a Gly Ala Ala Ile Gly
1825 1830
# 1835
# 1840
Ser Val Gly Leu Gly Lys Val Leu Val Asp Il
#e Leu Ala Gly Tyr Gly
                                1845
# 1850
# 1855
Ala Gly Val Ala Gly Ala Leu Val Ala Phe Ly
#s Ile Met Ser Gly Glu
                        1860
# 1865
# 1870
Val Pro Ser Thr Glu Asp Leu Val Asn Leu Le
#u Pro Ala Ile Leu Ser
                1875
# 1880
# 1885
Pro Gly Ala Leu Val Val Gly Val Val Cys Al
#a Ala Ile Leu Arg Arg
        1890
# 1895
# 1900
His Val Gly Pro Gly Glu Gly Ala Val Gln Tr
#p Met Asn Arg Leu Ile
1905 1910
# 1915
# 1920
Ala Phe Ala Ser Arg Gly Asn His Val Ser Pr
#o Thr His Tyr Val Pro
                                1925
# 1930
# 1935
Glu Ser Asp Ala Ala Ala Arg Val Thr Ala Il
#e Leu Ser Ser Leu Thr
                        1940
# 1945
# 1950
Val Thr Gln Leu Leu Arg Arg Leu His Gln Tr
#p Ile Ser Ser Glu Cys
                1955
# 1960
# 1965
Thr Thr Pro Cys Ser Gly Ser Trp Leu Arg As
#p Ile Trp Asp Trp Ile
        1970
# 1975
# 1980
Cys Glu Val Leu Ser Asp Phe Lys Thr Trp Le
#u Lys Ala Lys Leu Met
1985 1990
# 1995
# 2000
Pro Gln Leu Pro Gly Ile Pro Phe Val Ser Cy
#s Gln Arg Gly Tyr Arg
                                2005
# 2010
# 2015
Gly Val Trp Arg Gly Asp Gly Ile Met His Th
#r Arg Cys His Cys Gly
                        2020
# 2025
# 2030
Ala Glu Ile Thr Gly His Val Lys Asn Gly Th
#r Met Arg Ile Val Gly
                2035
# 2040
# 2045
Pro Arg Thr Cys Arg Asn Met Trp Ser Gly Th
#r Phe Pro Ile Asn Ala
        2050
# 2055
# 2060
Tyr Thr Thr Gly Pro Cys Thr Pro Leu Pro Al
#a Pro Asn Tyr Lys Phe
2065 2070
# 2075
# 2080
Ala Leu Trp Arg Val Ser Ala Glu Glu Tyr Va
#l Glu Ile Arg Arg Val
                                2085
# 2090
# 2095
Gly Asp Phe His Tyr Val Ser Gly Met Thr Th
#r Asp Asn Leu Lys Cys
                        2100
# 2105
# 2110
Pro Cys Gln Ile Pro Ser Pro Glu Phe Phe Th
#r Glu Leu Asp Gly Val
                2115
# 2120
# 2125
Arg Leu His Arg Phe Ala Pro Pro Cys Lys Pr
#o Leu Leu Arg Glu Glu
        2130
# 2135
# 2140
Val Ser Phe Arg Val Gly Leu His Glu Tyr Pr
#o Val Gly Ser Gln Leu
2145 2150
# 2155
# 2160
Pro Cys Glu Pro Glu Pro Asp Val Ala Val Le
#u Thr Ser Met Leu Thr
                                2165
# 2170
# 2175
Asp Pro Ser His Ile Thr Ala Glu Ala Ala Gl
#y Arg Arg Leu Ala Arg
                        2180
# 2185
# 2190
Gly Ser Pro Pro Ser Met Ala Ser Ser Ser Al
#a Ser Gln Leu Ser Ala
                2195
# 2200
# 2205
Pro Ser Leu Lys Ala Thr Cys Thr Ala Asn Hi
#s Asp Ser Pro Asp Ala
        2210
# 2215
# 2220
Glu Leu Ile Glu Ala Asn Leu Leu Trp Arg Gl
#n Glu Met Gly Gly Asn
2225 2230
# 2235
# 2240
Ile Thr Arg Val Glu Ser Glu Asn Lys Val Va
#l Ile Leu Asp Ser Phe
                                2245
# 2250
# 2255
Asp Pro Leu Val Ala Glu Glu Asp Glu Arg Gl
#u Val Ser Val Pro Ala
                        2260
# 2265
# 2270
Glu Ile Leu Arg Lys Ser Arg Arg Phe Ala Ar
#g Ala Leu Pro Val Trp
                2275
# 2280
# 2285
Ala Arg Pro Asp Tyr Asn Pro Pro Leu Val Gl
#u Thr Trp Lys Lys Pro
        2290
# 2295
# 2300
Asp Tyr Glu Pro Pro Val Val His Gly Cys Pr
#o Leu Pro Pro Pro Arg
2305 2310
# 2315
# 2320
Ser Pro Pro Val Pro Pro Pro Arg Lys Lys Ar
#g Thr Val Val Leu Thr
                                2325
# 2330
# 2335
Glu Ser Thr Leu Ser Thr Ala Leu Ala Glu Le
#u Ala Thr Lys Ser Phe
                        2340
# 2345
# 2350
Gly Ser Ser Ser Thr Ser Gly Ile Thr Gly As
#p Asn Thr Thr Thr Ser
                2355
# 2360
# 2365
Ser Glu Pro Ala Pro Ser Gly Cys Pro Pro As
#p Ser Asp Val Glu Ser
        2370
# 2375
# 2380
Tyr Ser Ser Met Pro Pro Leu Glu Gly Glu Pr
#o Gly Asp Pro Asp Leu
2385 2390
# 2395
# 2400
Ser Asp Gly Ser Trp Ser Thr Val Ser Ser Gl
#y Ala Asp Thr Glu Asp
                                2405
# 2410
# 2415
Val Val Cys Cys Ser Met Ser Tyr Ser Trp Th
#r Gly Ala Leu Val Thr
                        2420
# 2425
# 2430
Pro Cys Ala Ala Glu Glu Gln Lys Leu Pro Il
#e Asn Ala Leu Ser Asn
                2435
# 2440
# 2445
Ser Leu Leu Arg His His Asn Leu Val Tyr Se
#r Thr Thr Ser Arg Ser
        2450
# 2455
# 2460
Ala Cys Gln Arg Gln Lys Lys Val Thr Phe As
#p Arg Leu Gln Val Leu
2465 2470
# 2475
# 2480
Asp Ser His Tyr Gln Asp Val Leu Lys Glu Va
#l Lys Ala Ala Ala Ser
                                2485
# 2490
# 2495
Lys Val Lys Ala Asn Leu Leu Ser Val Glu Gl
#u Ala Cys Ser Leu Thr
                        2500
# 2505
# 2510
Pro Pro His Ser Ala Lys Ser Lys Phe Gly Ty
#r Gly Ala Lys Asp Val
                2515
# 2520
# 2525
Arg Cys His Ala Arg Lys Ala Val Ala His Il
#e Asn Ser Val Trp Lys
        2530
# 2535
# 2540
Asp Leu Leu Glu Asp Ser Val Thr Pro Ile As
#p Thr Thr Ile Met Ala
2545 2550
# 2555
# 2560
Lys Asn Glu Val Phe Cys Val Gln Pro Glu Ly
#s Gly Gly Arg Lys Pro
                                2565
# 2570
# 2575
Ala Arg Leu Ile Val Phe Pro Asp Leu Gly Va
#l Arg Val Cys Glu Lys
                        2580
# 2585
# 2590
Met Ala Leu Tyr Asp Val Val Ser Lys Leu Pr
#o Leu Ala Val Met Gly
                2595
# 2600
# 2605
Ser Ser Tyr Gly Phe Gln Tyr Ser Pro Gly Gl
#n Arg Val Glu Phe Leu
        2610
# 2615
# 2620
Val Gln Ala Trp Lys Ser Lys Lys Thr Pro Me
#t Gly Phe Ser Tyr Asp
2625 2630
# 2635
# 2640
Thr Arg Cys Phe Asp Ser Thr Val Thr Glu Se
#r Asp Ile Arg Thr Glu
                                2645
# 2650
# 2655
Glu Ala Ile Tyr Gln Cys Cys Asp Leu Asp Pr
#o Gln Ala Arg Val Ala
                        2660
# 2665
# 2670
Ile Lys Ser Leu Thr Glu Arg Leu Tyr Val Gl
#y Gly Pro Leu Thr Asn
                2675
# 2680
# 2685
Ser Arg Gly Glu Asn Cys Gly Tyr Arg Arg Cy
#s Arg Ala Ser Gly Val
        2690
# 2695
# 2700
Leu Thr Thr Ser Cys Gly Asn Thr Leu Thr Cy
#s Tyr Ile Lys Ala Arg
2705 2710
# 2715
# 2720
Ala Ala Cys Arg Ala Ala Gly Leu Gln Asp Cy
#s Thr Met Leu Val Cys
                                2725
# 2730
# 2735
Gly Asp Asp Leu Val Val Ile Cys Glu Ser Al
#a Gly Val Gln Glu Asp
                        2740
# 2745
# 2750
Ala Ala Ser Leu Arg Ala Phe Thr Glu Ala Me
#t Thr Arg Tyr Ser Ala
                2755
# 2760
# 2765
Pro Pro Gly Asp Pro Pro Gln Pro Glu Tyr As
#p Leu Glu Leu Ile Thr
        2770
# 2775
# 2780
Ser Cys Ser Ser Asn Val Ser Val Ala His As
#p Gly Ala Gly Lys Arg
2785 2790
# 2795
# 2800
Val Tyr Tyr Leu Thr Arg Asp Pro Thr Thr Pr
#o Leu Ala Arg Ala Ala
                                2805
# 2810
# 2815
Trp Glu Thr Ala Arg His Thr Pro Val Asn Se
#r Trp Leu Gly Asn Ile
                        2820
# 2825
# 2830
Ile Met Phe Ala Pro Thr Leu Trp Ala Arg Me
#t Ile Leu Met Thr His
                2835
# 2840
# 2845
Phe Phe Ser Val Leu Ile Ala Arg Asp Gln Le
#u Glu Gln Ala Leu Asn
        2850
# 2855
# 2860
Cys Glu Ile Tyr Gly Ala Cys Tyr Ser Ile Gl
#u Pro Leu Asp Leu Pro
2865 2870
# 2875
# 2880
Pro Ile Ile Gln Arg Leu His Gly Leu Ser Al
#a Phe Ser Leu His Ser
                                2885
# 2890
# 2895
Tyr Ser Pro Gly Glu Ile Asn Arg Val Ala Al
#a Cys Leu Arg Lys Leu
                        2900
# 2905
# 2910
Gly Val Pro Pro Leu Arg Ala Trp Arg His Ar
#g Ala Arg Ser Val Arg
                2915
# 2920
# 2925
Ala Arg Leu Leu Ser Arg Gly Gly Arg Ala Al
#a Ile Cys Gly Lys Tyr
        2930
# 2935
# 2940
Leu Phe Asn Trp Ala Val Arg Thr Lys Leu Ly
#s Leu Thr Pro Ile Ala
2945 2950
# 2955
# 2960
Ala Ala Gly Arg Leu Asp Leu Ser Gly Trp Ph
#e Thr Ala Gly Tyr Ser
                                2965
# 2970
# 2975
Gly Gly Asp Ile Tyr His Ser Val Ser His Al
#a Arg Pro Arg Trp Phe
                        2980
# 2985
# 2990
Trp Phe Cys Leu Leu Leu Leu Ala Ala Gly Va
#l Gly Ile Tyr Leu Leu
                2995
# 3000
# 3005
Pro Asn Arg
        3010
<210> SEQ ID NO 2
<211> LENGTH: 3033
<212> TYPE: PRT
<213> ORGANISM: Hepatitis C Virus
<400> SEQUENCE: 2
Met Ser Thr Asn Pro Lys Pro Gln Arg Lys Th
#r Lys Arg Asn Thr Asn
  1 5
# 10
# 15
Arg Arg Pro Glu Asp Val Lys Phe Pro Gly Gl
#y Gly Gln Ile Val Gly
                        20
# 25
# 30
Gly Val Tyr Leu Leu Pro Arg Arg Gly Pro Ar
#g Leu Gly Val Arg Thr
                35
# 40
# 45
Thr Arg Lys Thr Ser Glu Arg Ser Gln Pro Ar
#g Gly Arg Arg Gln Pro
        50
# 55
# 60
Ile Pro Lys Asp Arg Arg Ser Thr Gly Lys Al
#a Trp Gly Lys Pro Gly
65
#70
#75
#80
Arg Pro Trp Pro Leu Tyr Gly Asn Glu Gly Le
#u Gly Trp Ala Gly Trp
                                85
# 90
# 95
Leu Leu Ser Pro Arg Gly Ser Arg Pro Ser Tr
#p Gly Pro Thr Asp Pro
                        100
# 105
# 110
Arg His Arg Ser Arg Asn Val Gly Lys Val Il
#e Asp Thr Leu Thr Cys
                115
# 120
# 125
Gly Phe Ala Asp Leu Met Gly Tyr Ile Pro Va
#l Val Gly Ala Pro Leu
        130
# 135
# 140
Ser Gly Ala Ala Arg Ala Val Ala His Gly Va
#l Arg Val Leu Glu Asp
145 1
#50 1
#55 1
#60
Gly Val Asn Tyr Ala Thr Gly Asn Leu Pro Gl
#y Phe Pro Phe Ser Ile
                                165
# 170
# 175
Phe Leu Leu Ala Leu Leu Ser Cys Ile Thr Va
#l Pro Val Ser Ala Ala
                        180
# 185
# 190
Gln Val Lys Asn Thr Ser Ser Ser Tyr Met Va
#l Thr Asn Asp Cys Ser
                195
# 200
# 205
Asn Asp Ser Ile Thr Trp Gln Leu Glu Ala Al
#a Val Leu His Val Pro
        210
# 215
# 220
Gly Cys Val Pro Cys Glu Arg Val Gly Asn Th
#r Ser Arg Cys Trp Val
225 2
#30 2
#35 2
#40
Pro Val Ser Pro Asn Met Ala Val Arg Gln Pr
#o Gly Ala Leu Thr Gln
                                245
# 250
# 255
Gly Leu Arg Thr His Ile Asp Met Val Val Me
#t Ser Ala Thr Phe Cys
                        260
# 265
# 270
Ser Ala Leu Tyr Val Gly Asp Leu Cys Gly Gl
#y Val Met Leu Ala Ala
                275
# 280
# 285
Gln Val Phe Ile Val Ser Pro Gln Tyr His Tr
#p Phe Val Gln Glu Cys
        290
# 295
# 300
Asn Cys Ser Ile Tyr Pro Gly Thr Ile Thr Gl
#y His Arg Met Ala Trp
305 3
#10 3
#15 3
#20
Asp Met Met Met Asn Trp Ser Pro Thr Ala Th
#r Met Ile Leu Ala Tyr
                                325
# 330
# 335
Val Met Arg Val Pro Glu Val Ile Ile Asp Il
#e Val Ser Gly Ala His
                        340
# 345
# 350
Trp Gly Val Met Phe Gly Leu Ala Tyr Phe Se
#r Met Gln Gly Ala Trp
                355
# 360
# 365
Ala Lys Val Ile Val Ile Leu Leu Leu Ala Al
#a Gly Val Asp Ala Gly
        370
# 375
# 380
Thr Thr Thr Val Gly Gly Ala Val Ala Arg Se
#r Thr Asn Val Ile Ala
385 3
#90 3
#95 4
#00
Gly Val Phe Ser His Gly Pro Gln Gln Asn Il
#e Gln Leu Ile Asn Thr
                                405
# 410
# 415
Asn Gly Ser Trp His Ile Asn Arg Thr Ala Le
#u Asn Cys Asn Asp Ser
                        420
# 425
# 430
Leu Asn Thr Gly Phe Leu Ala Ala Leu Phe Ty
#r Thr Asn Arg Phe Asn
                435
# 440
# 445
Ser Ser Gly Cys Pro Gly Arg Leu Ser Ala Cy
#s Arg Asn Ile Glu Ala
        450
# 455
# 460
Phe Arg Ile Gly Trp Gly Thr Leu Gln Tyr Gl
#u Asp Asn Val Thr Asn
465 4
#70 4
#75 4
#80
Pro Glu Asp Met Arg Pro Tyr Cys Trp His Ty
#r Pro Pro Lys Pro Cys
                                485
# 490
# 495
Gly Val Val Pro Ala Arg Ser Val Cys Gly Pr
#o Val Tyr Cys Phe Thr
                        500
# 505
# 510
Pro Ser Pro Val Val Val Gly Thr Thr Asp Ar
#g Arg Gly Val Pro Thr
                515
# 520
# 525
Tyr Thr Trp Gly Glu Asn Glu Thr Asp Val Ph
#e Leu Leu Asn Ser Thr
        530
# 535
# 540
Arg Pro Pro Gln Gly Ser Trp Phe Gly Cys Th
#r Trp Met Asn Ser Thr
545 5
#50 5
#55 5
#60
Gly Phe Thr Lys Thr Cys Gly Ala Pro Pro Cy
#s Arg Thr Arg Ala Asp
                                565
# 570
# 575
Phe Asn Ala Ser Thr Asp Leu Leu Cys Pro Th
#r Asp Cys Phe Arg Lys
                        580
# 585
# 590
His Pro Asp Ala Thr Tyr Ile Lys Cys Gly Se
#r Gly Pro Trp Leu Thr
                595
# 600
# 605
Pro Lys Cys Leu Val His Tyr Pro Tyr Arg Le
#u Trp His Tyr Pro Cys
        610
# 615
# 620
Thr Val Asn Phe Thr Ile Phe Lys Ile Arg Me
#t Tyr Val Gly Gly Val
625 6
#30 6
#35 6
#40
Glu His Arg Leu Thr Ala Ala Cys Asn Phe Th
#r Arg Gly Asp Arg Cys
                                645
# 650
# 655
Asp Leu Glu Asp Arg Asp Arg Ser Gln Leu Se
#r Pro Leu Leu His Ser
                        660
# 665
# 670
Thr Thr Glu Trp Ala Ile Leu Pro Cys Thr Ty
#r Ser Asp Leu Pro Ala
                675
# 680
# 685
Leu Ser Thr Gly Leu Leu His Leu His Gln As
#n Ile Val Asp Val Gln
        690
# 695
# 700
Tyr Met Tyr Gly Leu Ser Pro Ala Ile Thr Ly
#s Tyr Val Val Arg Trp
705 7
#10 7
#15 7
#20
Glu Trp Val Val Leu Leu Phe Leu Leu Leu Al
#a Asp Ala Arg Val Cys
                                725
# 730
# 735
Ala Cys Leu Trp Met Leu Ile Leu Leu Gly Gl
#n Ala Glu Ala Ala Leu
                        740
# 745
# 750
Glu Lys Leu Val Val Leu His Ala Ala Ser Al
#a Ala Asn Cys His Gly
                755
# 760
# 765
Leu Leu Tyr Phe Ala Ile Phe Phe Val Ala Al
#a Trp His Ile Arg Gly
        770
# 775
# 780
Arg Val Val Pro Leu Thr Thr Tyr Cys Leu Th
#r Gly Leu Trp Pro Phe
785 7
#90 7
#95 8
#00
Cys Leu Leu Leu Met Ala Leu Pro Arg Gln Al
#a Tyr Ala Tyr Asp Ala
                                805
# 810
# 815
Pro Val His Gly Gln Ile Gly Val Gly Leu Le
#u Ile Leu Ile Thr Leu
                        820
# 825
# 830
Phe Thr Leu Thr Pro Gly Tyr Lys Thr Leu Le
#u Gly Gln Cys Leu Trp
                835
# 840
# 845
Trp Leu Cys Tyr Leu Leu Thr Leu Gly Glu Al
#a Met Ile Gln Glu Trp
        850
# 855
# 860
Val Pro Pro Met Gln Val Arg Gly Gly Arg As
#p Gly Ile Ala Trp Ala
865 8
#70 8
#75 8
#80
Val Thr Ile Phe Cys Pro Gly Val Val Phe As
#p Ile Thr Lys Trp Leu
                                885
# 890
# 895
Leu Ala Leu Leu Gly Pro Ala Tyr Leu Leu Ar
#g Ala Ala Leu Thr His
                        900
# 905
# 910
Val Pro Tyr Phe Val Arg Ala His Ala Leu Il
#e Arg Val Cys Ala Leu
                915
# 920
# 925
Val Lys Gln Leu Ala Gly Gly Arg Tyr Val Gl
#n Val Ala Leu Leu Ala
        930
# 935
# 940
Leu Gly Arg Trp Thr Gly Thr Tyr Ile Tyr As
#p His Leu Thr Pro Met
945 9
#50 9
#55 9
#60
Ser Asp Trp Ala Ala Ser Gly Leu Arg Asp Le
#u Ala Val Ala Val Glu
                                965
# 970
# 975
Pro Ile Ile Phe Ser Pro Met Glu Lys Lys Va
#l Ile Val Trp Gly Ala
                        980
# 985
# 990
Glu Thr Ala Ala Cys Gly Asp Ile Leu His Gl
#y Leu Pro Val Ser Ala
                995
# 1000
# 1005
Arg Leu Gly Gln Glu Ile Leu Leu Gly Pro Al
#a Asp Gly Tyr Thr Ser
        1010
# 1015
# 1020
Lys Gly Trp Lys Leu Leu Ala Pro Ile Thr Al
#a Tyr Ala Gln Gln Thr
1025 1030
# 1035
# 1040
Arg Gly Leu Leu Gly Ala Ile Val Val Ser Me
#t Thr Gly Arg Asp Arg
                                1045
# 1050
# 1055
Thr Glu Gln Ala Gly Glu Val Gln Ile Leu Se
#r Thr Val Ser Gln Ser
                        1060
# 1065
# 1070
Phe Leu Gly Thr Thr Ile Ser Gly Val Leu Tr
#p Thr Val Tyr His Gly
                1075
# 1080
# 1085
Ala Gly Asn Lys Thr Leu Ala Gly Leu Arg Gl
#y Pro Val Thr Gln Met
        1090
# 1095
# 1100
Tyr Ser Ser Ala Glu Gly Asp Leu Val Gly Tr
#p Pro Ser Pro Pro Gly
1105 1110
# 1115
# 1120
Thr Lys Ser Leu Glu Pro Cys Lys Cys Gly Al
#a Val Asp Leu Tyr Leu
                                1125
# 1130
# 1135
Val Thr Arg Asn Ala Asp Val Ile Pro Ala Ar
#g Arg Arg Gly Asp Lys
                        1140
# 1145
# 1150
Arg Gly Ala Leu Leu Ser Pro Arg Pro Ile Se
#r Thr Leu Lys Gly Ser
                1155
# 1160
# 1165
Ser Gly Gly Pro Val Leu Cys Pro Arg Gly Hi
#s Val Val Gly Leu Phe
        1170
# 1175
# 1180
Arg Ala Ala Val Cys Ser Arg Gly Val Ala Ly
#s Ser Ile Asp Phe Ile
1185 1190
# 1195
# 1200
Pro Val Glu Thr Leu Asp Val Val Thr Arg Se
#r Pro Thr Phe Ser Asp
                                1205
# 1210
# 1215
Asn Ser Thr Pro Pro Ala Val Pro Gln Thr Ty
#r Gln Val Gly Tyr Leu
                        1220
# 1225
# 1230
His Ala Pro Thr Gly Ser Gly Lys Ser Thr Ly
#s Val Pro Val Ala Tyr
                1235
# 1240
# 1245
Ala Ala Gln Gly Tyr Lys Val Leu Val Leu As
#n Pro Ser Val Ala Ala
        1250
# 1255
# 1260
Thr Leu Gly Phe Gly Ala Tyr Leu Ser Lys Al
#a His Gly Ile Asn Pro
1265 1270
# 1275
# 1280
Asn Ile Arg Thr Gly Val Arg Thr Val Met Th
#r Gly Glu Ala Ile Thr
                                1285
# 1290
# 1295
Tyr Ser Thr Tyr Gly Lys Phe Leu Ala Asp Gl
#y Gly Cys Ala Ser Gly
                        1300
# 1305
# 1310
Ala Tyr Asp Ile Ile Ile Cys Asp Glu Cys Hi
#s Ala Val Asp Ala Thr
                1315
# 1320
# 1325
Ser Ile Leu Gly Ile Gly Thr Val Leu Asp Gl
#n Ala Glu Thr Ala Gly
        1330
# 1335
# 1340
Val Arg Leu Thr Val Leu Ala Thr Ala Thr Pr
#o Pro Gly Ser Val Thr
1345 1350
# 1355
# 1360
Thr Pro His Pro Asp Ile Glu Glu Val Gly Le
#u Gly Arg Glu Gly Glu
                                1365
# 1370
# 1375
Ile Pro Phe Tyr Gly Arg Ala Ile Pro Leu Se
#r Cys Ile Lys Gly Gly
                        1380
# 1385
# 1390
Arg His Leu Ile Phe Cys His Ser Lys Lys Ly
#s Cys Asp Glu Leu Ala
                1395
# 1400
# 1405
Ala Ala Leu Arg Gly Met Gly Leu Asn Ala Va
#l Ala Tyr Tyr Arg Gly
        1410
# 1415
# 1420
Leu Asp Val Ser Ile Ile Pro Ala Gln Gly As
#p Val Val Val Val Ala
1425 1430
# 1435
# 1440
Thr Asp Ala Leu Met Thr Gly Tyr Thr Gly As
#p Phe Asp Ser Val Ile
                                1445
# 1450
# 1455
Asp Cys Asn Val Ala Val Thr Gln Ala Val As
#p Phe Ser Leu Asp Pro
                        1460
# 1465
# 1470
Thr Phe Thr Ile Thr Thr Gln Thr Val Pro Gl
#n Asp Ala Val Ser Arg
                1475
# 1480
# 1485
Ser Gln Arg Arg Gly Arg Thr Gly Arg Gly Ar
#g Gln Gly Thr Tyr Arg
        1490
# 1495
# 1500
Tyr Val Ser Thr Gly Glu Arg Ala Ser Gly Me
#t Phe Asp Ser Val Val
1505 1510
# 1515
# 1520
Leu Cys Glu Cys Tyr Asp Ala Gly Ala Ala Tr
#p Tyr Asp Leu Thr Pro
                                1525
# 1530
# 1535
Ala Glu Thr Thr Val Arg Leu Arg Ala Tyr Ph
#e Asn Thr Pro Gly Leu
                        1540
# 1545
# 1550
Pro Val Cys Gln Asp His Leu Glu Phe Trp Gl
#u Ala Val Phe Thr Gly
                1555
# 1560
# 1565
Leu Thr His Ile Asp Ala His Phe Leu Ser Gl
#n Thr Lys Gln Ala Gly
        1570
# 1575
# 1580
Glu Asn Phe Ala Tyr Leu Val Ala Tyr Gln Al
#a Thr Val Cys Ala Arg
1585 1590
# 1595
# 1600
Ala Lys Ala Pro Pro Pro Ser Trp Asp Ala Me
#t Trp Lys Cys Leu Ala
                                1605
# 1610
# 1615
Arg Leu Lys Pro Thr Leu Ala Gly Pro Thr Pr
#o Leu Leu Tyr Arg Leu
                        1620
# 1625
# 1630
Gly Pro Ile Thr Asn Glu Val Thr Leu Thr Hi
#s Pro Gly Thr Lys Tyr
                1635
# 1640
# 1645
Ile Ala Thr Cys Met Gln Ala Asp Leu Glu Va
#l Met Thr Ser Thr Trp
        1650
# 1655
# 1660
Val Leu Ala Gly Gly Val Leu Ala Ala Val Al
#a Ala Tyr Cys Leu Ala
1665 1670
# 1675
# 1680
Thr Gly Cys Val Ser Ile Ile Gly Arg Leu Hi
#s Val Asn Gln Arg Val
                                1685
# 1690
# 1695
Val Val Ala Pro Asp Lys Glu Val Leu Tyr Gl
#u Ala Phe Asp Glu Met
                        1700
# 1705
# 1710
Glu Glu Cys Ala Ser Arg Ala Ala Leu Ile Gl
#u Glu Gly Gln Arg Ile
                1715
# 1720
# 1725
Ala Glu Met Leu Lys Ser Lys Ile Gln Gly Le
#u Leu Gln Gln Ala Ser
        1730
# 1735
# 1740
Lys Gln Ala Gln Asp Ile Gln Pro Ala Met Gl
#n Ala Ser Trp Pro Lys
1745 1750
# 1755
# 1760
Val Glu Gln Phe Trp Ala Arg His Met Trp As
#n Phe Ile Ser Gly Ile
                                1765
# 1770
# 1775
Gln Tyr Leu Ala Gly Leu Ser Thr Leu Pro Gl
#y Asn Pro Ala Val Ala
                        1780
# 1785
# 1790
Ser Met Met Ala Phe Ser Ala Ala Leu Thr Se
#r Pro Leu Ser Thr Ser
                1795
# 1800
# 1805
Thr Thr Ile Leu Leu Asn Ile Met Gly Gly Tr
#p Leu Ala Ser Gln Ile
        1810
# 1815
# 1820
Ala Pro Pro Ala Gly Ala Thr Gly Phe Val Va
#l Ser Gly Leu Val Gly
1825 1830
# 1835
# 1840
Ala Ala Val Gly Ser Ile Gly Leu Gly Lys Va
#l Leu Val Asp Ile Leu
                                1845
# 1850
# 1855
Ala Gly Tyr Gly Ala Gly Ile Ser Gly Ala Le
#u Val Ala Phe Lys Ile
                        1860
# 1865
# 1870
Met Ser Gly Glu Lys Pro Ser Met Glu Asp Va
#l Ile Asn Leu Leu Pro
                1875
# 1880
# 1885
Gly Ile Leu Ser Pro Gly Ala Leu Val Val Gl
#y Val Ile Cys Ala Ala
        1890
# 1895
# 1900
Ile Leu Arg Arg His Val Gly Pro Gly Glu Gl
#y Ala Val Gln Trp Met
1905 1910
# 1915
# 1920
Asn Arg Leu Ile Ala Phe Ala Ser Arg Gly As
#n His Val Ala Pro Thr
                                1925
# 1930
# 1935
His Tyr Val Thr Glu Ser Asp Ala Ser Gln Ar
#g Val Thr Gln Leu Leu
                        1940
# 1945
# 1950
Gly Ser Leu Thr Ile Thr Ser Leu Leu Arg Ar
#g Leu His Asn Trp Ile
                1955
# 1960
# 1965
Thr Glu Asp Cys Pro Ile Pro Cys Ser Gly Se
#r Trp Leu Arg Asp Val
        1970
# 1975
# 1980
Trp Asp Trp Val Cys Thr Ile Leu Thr Asp Ph
#e Lys Asn Trp Leu Thr
1985 1990
# 1995
# 2000
Ser Lys Leu Phe Pro Lys Leu Pro Gly Leu Pr
#o Phe Ile Ser Cys Gln
                                2005
# 2010
# 2015
Lys Gly Tyr Lys Gly Val Trp Ala Gly Thr Gl
#y Ile Met Thr Thr Arg
                        2020
# 2025
# 2030
Cys Pro Cys Gly Ala Asn Ile Ser Gly Asn Va
#l Arg Leu Gly Ser Met
                2035
# 2040
# 2045
Arg Ile Thr Gly Pro Lys Thr Cys Met Asn Th
#r Trp Gln Gly Thr Phe
        2050
# 2055
# 2060
Pro Ile Asn Cys Tyr Thr Glu Gly Gln Cys Al
#a Pro Lys Pro Pro Thr
2065 2070
# 2075
# 2080
Asn Tyr Lys Thr Ala Ile Trp Arg Val Ala Al
#a Ser Glu Tyr Ala Glu
                                2085
# 2090
# 2095
Val Thr Gln His Gly Ser Tyr Ser Tyr Val Th
#r Gly Leu Thr Thr Asp
                        2100
# 2105
# 2110
Asn Leu Lys Ile Pro Cys Gln Leu Pro Ser Pr
#o Glu Phe Phe Ser Trp
                2115
# 2120
# 2125
Val Asp Gly Val Gln Ile His Arg Phe Ala Pr
#o Thr Pro Lys Pro Phe
        2130
# 2135
# 2140
Phe Arg Asp Glu Val Ser Phe Cys Val Gly Le
#u Asn Ser Tyr Ala Val
2145 2150
# 2155
# 2160
Gly Ser Gln Leu Pro Cys Glu Pro Glu Pro As
#p Ala Asp Val Leu Arg
                                2165
# 2170
# 2175
Ser Met Leu Thr Asp Pro Pro His Ile Thr Al
#a Glu Thr Ala Ala Arg
                        2180
# 2185
# 2190
Arg Leu Ala Arg Gly Ser Pro Pro Ser Glu Al
#a Ser Ser Ser Val Ser
                2195
# 2200
# 2205
Gln Leu Ser Ala Pro Ser Leu Arg Ala Thr Cy
#s Thr Thr His Ser Asn
        2210
# 2215
# 2220
Thr Tyr Asp Val Asp Met Val Asp Ala Asn Le
#u Leu Met Glu Gly Gly
2225 2230
# 2235
# 2240
Val Ala Gln Thr Glu Pro Glu Ser Arg Val Pr
#o Val Leu Asp Phe Leu
                                2245
# 2250
# 2255
Glu Pro Met Ala Glu Glu Glu Ser Asp Leu Gl
#u Pro Ser Ile Pro Ser
                        2260
# 2265
# 2270
Glu Cys Met Leu Pro Arg Ser Gly Phe Pro Ar
#g Ala Leu Pro Ala Trp
                2275
# 2280
# 2285
Ala Arg Pro Asp Tyr Asn Pro Pro Leu Val Gl
#u Ser Trp Arg Arg Pro
        2290
# 2295
# 2300
Asp Tyr Gln Pro Pro Thr Val Ala Gly Cys Al
#a Leu Pro Pro Pro Lys
2305 2310
# 2315
# 2320
Lys Ala Pro Thr Pro Pro Pro Arg Arg Arg Ar
#g Thr Val Gly Leu Ser
                                2325
# 2330
# 2335
Glu Ser Thr Ile Ser Glu Ala Leu Gln Gln Le
#u Ala Ile Lys Thr Phe
                        2340
# 2345
# 2350
Gly Gln Pro Pro Ser Ser Gly Asp Ala Gly Se
#r Ser Thr Gly Ala Gly
                2355
# 2360
# 2365
Ala Ala Glu Ser Gly Gly Pro Thr Ser Pro Gl
#y Glu Pro Ala Pro Ser
        2370
# 2375
# 2380
Glu Thr Gly Ser Ala Ser Ser Met Pro Pro Le
#u Glu Gly Glu Pro Gly
2385 2390
# 2395
# 2400
Asp Pro Asp Leu Glu Ser Asp Gln Val Glu Le
#u Gln Pro Pro Pro Gln
                                2405
# 2410
# 2415
Gly Gly Gly Val Ala Pro Gly Ser Gly Ser Gl
#y Ser Trp Ser Thr Cys
                        2420
# 2425
# 2430
Ser Glu Glu Asp Asp Thr Thr Val Cys Cys Se
#r Met Ser Tyr Ser Trp
                2435
# 2440
# 2445
Thr Gly Ala Leu Ile Thr Pro Cys Ser Pro Gl
#u Glu Glu Lys Leu Pro
        2450
# 2455
# 2460
Ile Asn Pro Leu Ser Asn Ser Leu Leu Arg Ty
#r His Asn Lys Val Tyr
2465 2470
# 2475
# 2480
Cys Thr Thr Ser Lys Ser Ala Ser Gln Arg Al
#a Lys Lys Val Thr Phe
                                2485
# 2490
# 2495
Asp Arg Thr Gln Val Leu Asp Ala His Tyr As
#p Ser Val Leu Lys Asp
                        2500
# 2505
# 2510
Ile Lys Leu Ala Ala Ser Lys Val Ser Ala Ar
#g Leu Leu Thr Leu Glu
                2515
# 2520
# 2525
Glu Ala Cys Gln Leu Thr Pro Pro His Ser Al
#a Arg Ser Lys Tyr Gly
        2530
# 2535
# 2540
Phe Gly Ala Lys Glu Val Arg Ser Leu Ser Gl
#y Arg Ala Val Asn His
2545 2550
# 2555
# 2560
Ile Lys Ser Val Trp Lys Asp Leu Leu Glu As
#p Pro Gln Thr Pro Ile
                                2565
# 2570
# 2575
Pro Thr Thr Ile Met Ala Lys Asn Glu Val Ph
#e Cys Val Asp Pro Ala
                        2580
# 2585
# 2590
Lys Gly Gly Lys Lys Pro Ala Arg Leu Ile Va
#l Tyr Pro Asp Leu Gly
                2595
# 2600
# 2605
Val Arg Val Cys Glu Lys Met Ala Leu Tyr As
#p Ile Thr Gln Lys Leu
        2610
# 2615
# 2620
Pro Gln Ala Val Met Gly Ala Ser Tyr Gly Ph
#e Gln Tyr Ser Pro Ala
2625 2630
# 2635
# 2640
Gln Arg Val Glu Tyr Leu Leu Lys Ala Trp Al
#a Glu Lys Lys Asp Pro
                                2645
# 2650
# 2655
Met Gly Phe Ser Tyr Asp Thr Arg Cys Phe As
#p Ser Thr Val Thr Glu
                        2660
# 2665
# 2670
Arg Asp Ile Arg Thr Glu Glu Ser Ile Tyr Gl
#n Ala Cys Ser Leu Pro
                2675
# 2680
# 2685
Glu Glu Ala Arg Thr Ala Ile His Ser Leu Th
#r Glu Arg Leu Tyr Val
        2690
# 2695
# 2700
Gly Gly Pro Met Phe Asn Ser Lys Gly Gln Th
#r Cys Gly Tyr Arg Arg
2705 2710
# 2715
# 2720
Cys Arg Ala Ser Gly Val Leu Thr Thr Ser Me
#t Gly Asn Thr Ile Thr
                                2725
# 2730
# 2735
Cys Tyr Val Lys Ala Leu Ala Ala Cys Lys Al
#a Ala Gly Ile Val Ala
                        2740
# 2745
# 2750
Pro Thr Met Leu Val Cys Gly Asp Asp Leu Va
#l Val Ile Ser Glu Ser
                2755
# 2760
# 2765
Gln Gly Thr Glu Glu Asp Glu Arg Asn Leu Ar
#g Ala Phe Thr Glu Ala
        2770
# 2775
# 2780
Met Thr Arg Tyr Ser Ala Pro Pro Gly Asp Pr
#o Pro Arg Pro Glu Tyr
2785 2790
# 2795
# 2800
Asp Leu Glu Leu Ile Thr Ser Cys Ser Ser As
#n Val Ser Val Ala Leu
                                2805
# 2810
# 2815
Gly Pro Arg Gly Arg Arg Arg Tyr Tyr Leu Th
#r Arg Asp Pro Thr Thr
                        2820
# 2825
# 2830
Pro Leu Ala Arg Ala Ala Trp Glu Thr Val Ar
#g His Ser Pro Ile Asn
                2835
# 2840
# 2845
Ser Trp Leu Gly Asn Ile Ile Gln Tyr Ala Pr
#o Thr Ile Trp Val Arg
        2850
# 2855
# 2860
Met Val Leu Met Thr His Phe Phe Ser Ile Le
#u Met Val Gln Asp Thr
2865 2870
# 2875
# 2880
Leu Asp Gln Asn Leu Asn Phe Glu Met Tyr Gl
#y Ser Val Tyr Ser Val
                                2885
# 2890
# 2895
Asn Pro Leu Asp Leu Pro Ala Ile Ile Glu Ar
#g Leu His Gly Leu Asp
                        2900
# 2905
# 2910
Ala Phe Ser Met His Thr Tyr Ser His His Gl
#u Leu Thr Arg Val Ala
                2915
# 2920
# 2925
Ser Ala Leu Arg Lys Leu Gly Ala Pro Pro Le
#u Arg Val Trp Lys Ser
        2930
# 2935
# 2940
Arg Ala Arg Ala Val Arg Ala Ser Leu Ile Se
#r Arg Gly Gly Lys Ala
2945 2950
# 2955
# 2960
Ala Val Cys Gly Arg Tyr Leu Phe Asn Trp Al
#a Val Lys Thr Lys Leu
                                2965
# 2970
# 2975
Lys Leu Thr Pro Leu Pro Glu Ala Arg Leu Le
#u Asp Leu Ser Ser Trp
                        2980
# 2985
# 2990
Phe Thr Val Gly Ala Gly Gly Gly Asp Ile Ph
#e His Ser Val Ser Arg
                2995
# 3000
# 3005
Ala Arg Pro Arg Ser Leu Leu Phe Gly Leu Le
#u Leu Leu Phe Val Gly
        3010
# 3015
# 3020
Val Gly Leu Phe Leu Leu Pro Ala Arg
3025 3030
<210> SEQ ID NO 3
<211> LENGTH: 3010
<212> TYPE: PRT
<213> ORGANISM: Hepatitis C Virus
<400> SEQUENCE: 3
Met Ser Thr Asn Pro Lys Pro Gln Arg Lys Th
#r Lys Arg Asn Thr Asn
  1 5
# 10
# 15
Arg Arg Pro Gln Asp Val Lys Phe Pro Gly Gl
#y Gly Gln Ile Val Gly
                        20
# 25
# 30
Gly Val Tyr Leu Leu Pro Arg Arg Gly Pro Ar
#g Leu Gly Val Arg Ala
                35
# 40
# 45
Thr Arg Lys Thr Ser Glu Arg Ser Gln Pro Ar
#g Gly Arg Arg Gln Pro
        50
# 55
# 60
Ile Pro Lys Ala Arg Gln Pro Glu Gly Arg Al
#a Trp Ala Gln Pro Gly
65
#70
#75
#80
Tyr Pro Trp Pro Leu Tyr Gly Asn Glu Gly Le
#u Gly Trp Ala Gly Trp
                                85
# 90
# 95
Leu Leu Ser Pro Arg Gly Ser Arg Pro Ser Tr
#p Gly Pro Thr Asp Pro
                        100
# 105
# 110
Arg Arg Arg Ser Arg Asn Leu Gly Lys Val Il
#e Asp Thr Leu Thr Cys
                115
# 120
# 125
Gly Phe Ala Asp Leu Met Gly Tyr Ile Pro Le
#u Val Gly Ala Pro Leu
        130
# 135
# 140
Gly Gly Ala Ala Arg Ala Leu Ala His Gly Va
#l Arg Val Leu Glu Asp
145 1
#50 1
#55 1
#60
Gly Val Asn Tyr Ala Thr Gly Asn Leu Pro Gl
#y Cys Ser Phe Ser Ile
                                165
# 170
# 175
Phe Leu Leu Ala Leu Leu Ser Cys Leu Thr Il
#e Pro Ala Ser Ala Tyr
                        180
# 185
# 190
Glu Val Arg Asn Val Ser Gly Val Tyr His Va
#l Thr Asn Asp Cys Ser
                195
# 200
# 205
Asn Ala Ser Ile Val Tyr Glu Ala Ala Asp Me
#t Ile Met His Thr Pro
        210
# 215
# 220
Gly Cys Val Pro Cys Val Arg Glu Asn Asn Se
#r Ser Arg Cys Trp Val
225 2
#30 2
#35 2
#40
Ala Leu Thr Pro Thr Leu Ala Ala Arg Asn Al
#a Ser Val Pro Thr Thr
                                245
# 250
# 255
Thr Ile Arg Arg His Val Asp Leu Leu Val Gl
#y Ala Ala Ala Leu Cys
                        260
# 265
# 270
Ser Ala Met Tyr Val Gly Asp Leu Cys Gly Se
#r Val Phe Leu Val Ala
                275
# 280
# 285
Gln Leu Phe Thr Phe Ser Pro Arg Arg His Gl
#u Thr Val Gln Asp Cys
        290
# 295
# 300
Asn Cys Ser Ile Tyr Pro Gly His Val Thr Gl
#y His Arg Met Ala Trp
305 3
#10 3
#15 3
#20
Asp Met Met Met Asn Trp Ser Pro Thr Ala Al
#a Leu Val Val Ser Gln
                                325
# 330
# 335
Leu Leu Arg Ile Pro Gln Ala Val Val Asp Me
#t Val Ala Gly Ala His
                        340
# 345
# 350
Trp Gly Val Leu Ala Gly Leu Ala Tyr Tyr Se
#r Met Val Gly Asn Trp
                355
# 360
# 365
Ala Lys Val Leu Ile Val Met Leu Leu Phe Al
#a Gly Val Asp Gly Gly
        370
# 375
# 380
Thr Tyr Val Thr Gly Gly Thr Met Ala Lys As
#n Thr Leu Gly Ile Thr
385 3
#90 3
#95 4
#00
Ser Leu Phe Ser Pro Gly Ser Ser Gln Lys Il
#e Gln Leu Val Asn Thr
                                405
# 410
# 415
Asn Gly Ser Trp His Ile Asn Arg Thr Ala Le
#u Asn Cys Asn Asp Ser
                        420
# 425
# 430
Leu Asn Thr Gly Phe Leu Ala Ala Leu Phe Ty
#r Val His Lys Phe Asn
                435
# 440
# 445
Ser Ser Gly Cys Pro Glu Arg Met Ala Ser Cy
#s Ser Pro Ile Asp Ala
        450
# 455
# 460
Phe Ala Gln Gly Trp Gly Pro Ile Thr Tyr As
#n Glu Ser His Ser Ser
465 4
#70 4
#75 4
#80
Asp Gln Arg Pro Tyr Cys Trp His Tyr Ala Pr
#o Arg Pro Cys Gly Ile
                                485
# 490
# 495
Val Pro Ala Ala Gln Val Cys Gly Pro Val Ty
#r Cys Phe Thr Pro Ser
                        500
# 505
# 510
Pro Val Val Val Gly Thr Thr Asp Arg Phe Gl
#y Val Pro Thr Tyr Ser
                515
# 520
# 525
Trp Gly Glu Asn Glu Thr Asp Val Leu Leu Le
#u Asn Asn Thr Arg Pro
        530
# 535
# 540
Pro Gln Gly Asn Trp Phe Gly Cys Thr Trp Me
#t Asn Ser Thr Gly Phe
545 5
#50 5
#55 5
#60
Thr Lys Thr Cys Gly Gly Pro Pro Cys Asn Il
#e Gly Gly Ile Gly Asn
                                565
# 570
# 575
Lys Thr Leu Thr Cys Pro Thr Asp Cys Phe Ar
#g Lys His Pro Glu Ala
                        580
# 585
# 590
Thr Tyr Thr Lys Cys Gly Ser Gly Pro Trp Le
#u Thr Pro Arg Cys Leu
                595
# 600
# 605
Val His Tyr Pro Tyr Arg Leu Trp His Tyr Pr
#o Cys Thr Val Asn Phe
        610
# 615
# 620
Thr Ile Phe Lys Val Arg Met Tyr Val Gly Gl
#y Val Glu His Arg Leu
625 6
#30 6
#35 6
#40
Glu Ala Ala Cys Asn Trp Thr Arg Gly Glu Ar
#g Cys Asn Leu Glu Asp
                                645
# 650
# 655
Arg Asp Arg Ser Glu Leu Ser Pro Leu Leu Le
#u Ser Thr Thr Glu Trp
                        660
# 665
# 670
Gln Val Leu Pro Cys Ser Phe Thr Thr Leu Pr
#o Ala Leu Ser Thr Gly
                675
# 680
# 685
Leu Ile His Leu His Gln Asn Val Val Asp Va
#l Gln Tyr Leu Tyr Gly
        690
# 695
# 700
Ile Gly Ser Ala Val Val Ser Phe Ala Ile Ly
#s Trp Glu Tyr Val Leu
705 7
#10 7
#15 7
#20
Leu Leu Phe Leu Leu Leu Ala Asp Ala Arg Va
#l Cys Ala Cys Leu Trp
                                725
# 730
# 735
Met Met Leu Leu Ile Ala Gln Ala Glu Ala Al
#a Leu Glu Asn Leu Val
                        740
# 745
# 750
Val Leu Asn Ala Ala Ser Val Ala Gly Ala Hi
#s Gly Ile Leu Ser Phe
                755
# 760
# 765
Leu Val Phe Phe Cys Ala Ala Trp Tyr Ile Ly
#s Gly Arg Leu Val Pro
        770
# 775
# 780
Gly Ala Ala Tyr Ala Leu Tyr Gly Val Trp Pr
#o Leu Leu Leu Leu Leu
785 7
#90 7
#95 8
#00
Leu Ala Leu Pro Pro Arg Ala Tyr Ala Met As
#p Arg Glu Met Ala Ala
                                805
# 810
# 815
Ser Cys Gly Gly Ala Val Phe Val Gly Leu Il
#e Leu Leu Thr Leu Ser
                        820
# 825
# 830
Pro His Tyr Lys Leu Phe Leu Ala Arg Leu Il
#e Trp Trp Leu Gln Tyr
                835
# 840
# 845
Phe Ile Thr Arg Ala Glu Ala His Leu Gln Va
#l Trp Ile Pro Pro Leu
        850
# 855
# 860
Asn Val Arg Gly Gly Arg Asp Ala Val Ile Le
#u Leu Thr Cys Ala Ile
865 8
#70 8
#75 8
#80
His Pro Glu Leu Ile Phe Thr Ile Thr Lys Il
#e Leu Leu Ala Ile Leu
                                885
# 890
# 895
Gly Pro Leu Met Val Leu Gln Ala Gly Ile Th
#r Lys Val Pro Tyr Phe
                        900
# 905
# 910
Val Arg Ala His Gly Leu Ile Arg Ala Cys Me
#t Leu Val Arg Lys Val
                915
# 920
# 925
Ala Gly Gly His Tyr Val Gln Met Ala Leu Me
#t Lys Leu Ala Ala Leu
        930
# 935
# 940
Thr Gly Thr Tyr Val Tyr Asp His Leu Thr Pr
#o Leu Arg Asp Trp Ala
945 9
#50 9
#55 9
#60
His Ala Gly Leu Arg Asp Leu Ala Val Ala Va
#l Glu Pro Val Val Phe
                                965
# 970
# 975
Ser Asp Met Glu Thr Lys Val Ile Thr Trp Gl
#y Ala Asp Thr Ala Ala
                        980
# 985
# 990
Cys Gly Asp Ile Ile Leu Gly Leu Pro Val Se
#r Ala Arg Arg Gly Arg
                995
# 1000
# 1005
Glu Ile His Leu Gly Pro Ala Asp Ser Leu Gl
#u Gly Gln Gly Trp Arg
        1010
# 1015
# 1020
Leu Leu Ala Pro Ile Thr Ala Tyr Ser Gln Gl
#n Thr Arg Gly Leu Leu
1025 1030
# 1035
# 1040
Gly Cys Ile Ile Thr Ser Leu Thr Gly Arg As
#p Arg Asn Gln Val Glu
                                1045
# 1050
# 1055
Gly Glu Val Gln Val Val Ser Thr Ala Thr Gl
#n Ser Phe Leu Ala Thr
                        1060
# 1065
# 1070
Cys Val Asn Gly Val Cys Trp Thr Val Tyr Hi
#s Gly Ala Gly Ser Lys
                1075
# 1080
# 1085
Thr Leu Ala Gly Pro Lys Gly Pro Ile Thr Gl
#n Met Tyr Thr Asn Val
        1090
# 1095
# 1100
Asp Gln Asp Leu Val Gly Trp Gln Ala Pro Pr
#o Gly Ala Arg Ser Leu
1105 1110
# 1115
# 1120
Thr Pro Cys Thr Cys Gly Ser Ser Asp Leu Ty
#r Leu Val Thr Arg His
                                1125
# 1130
# 1135
Ala Asp Val Ile Pro Val Arg Arg Arg Gly As
#p Ser Arg Gly Ser Leu
                        1140
# 1145
# 1150
Leu Ser Pro Arg Pro Val Ser Tyr Leu Lys Gl
#y Ser Ser Gly Gly Pro
                1155
# 1160
# 1165
Leu Leu Cys Pro Ser Gly His Ala Val Gly Il
#e Phe Arg Ala Ala Val
        1170
# 1175
# 1180
Cys Thr Arg Gly Val Ala Lys Ala Val Asp Ph
#e Val Pro Val Glu Ser
1185 1190
# 1195
# 1200
Met Glu Thr Thr Met Arg Ser Pro Val Phe Th
#r Asp Asn Ser Ser Pro
                                1205
# 1210
# 1215
Pro Ala Val Pro Gln Thr Phe Gln Val Ala Hi
#s Leu His Ala Pro Thr
                        1220
# 1225
# 1230
Gly Ser Gly Lys Ser Thr Lys Val Pro Ala Al
#a Tyr Ala Ala Gln Gly
                1235
# 1240
# 1245
Tyr Lys Val Leu Val Leu Asn Pro Ser Val Al
#a Ala Thr Leu Gly Phe
        1250
# 1255
# 1260
Gly Ala Tyr Met Ser Lys Ala His Gly Ile As
#p Pro Asn Ile Arg Thr
1265 1270
# 1275
# 1280
Gly Val Arg Thr Ile Thr Thr Gly Ala Pro Il
#e Thr Tyr Ser Thr Tyr
                                1285
# 1290
# 1295
Gly Lys Phe Leu Ala Asp Gly Gly Cys Ser Gl
#y Gly Ala Tyr Asp Ile
                        1300
# 1305
# 1310
Ile Ile Cys Asp Glu Cys His Ser Thr Asp Se
#r Thr Thr Ile Leu Gly
                1315
# 1320
# 1325
Ile Gly Thr Val Leu Asp Gln Ala Glu Thr Al
#a Gly Ala Arg Leu Val
        1330
# 1335
# 1340
Val Leu Ala Thr Ala Thr Pro Pro Gly Ser Va
#l Thr Val Pro His Pro
1345 1350
# 1355
# 1360
Asn Ile Glu Glu Val Ala Leu Ser Ser Thr Gl
#y Glu Ile Pro Phe Tyr
                                1365
# 1370
# 1375
Gly Lys Ala Ile Pro Ile Glu Thr Ile Lys Gl
#y Gly Arg His Leu Ile
                        1380
# 1385
# 1390
Phe Cys His Ser Lys Lys Lys Cys Asp Glu Le
#u Ala Ala Lys Leu Ser
                1395
# 1400
# 1405
Gly Leu Gly Leu Asn Ala Val Ala Tyr Tyr Ar
#g Gly Leu Asp Val Ser
        1410
# 1415
# 1420
Val Ile Pro Thr Ser Gly Asp Val Ile Val Va
#l Ala Thr Asp Ala Leu
1425 1430
# 1435
# 1440
Met Thr Gly Phe Thr Gly Asp Phe Asp Ser Va
#l Ile Asp Cys Asn Thr
                                1445
# 1450
# 1455
Cys Val Thr Gln Thr Val Asp Phe Ser Leu As
#p Pro Thr Phe Thr Ile
                        1460
# 1465
# 1470
Glu Thr Thr Thr Val Pro Gln Asp Ala Val Se
#r Arg Ser Gln Arg Arg
                1475
# 1480
# 1485
Gly Arg Thr Gly Arg Gly Arg Met Gly Ile Ty
#r Arg Phe Val Thr Pro
        1490
# 1495
# 1500
Gly Glu Arg Pro Ser Gly Met Phe Asp Ser Se
#r Val Leu Cys Glu Cys
1505 1510
# 1515
# 1520
Tyr Asp Ala Gly Cys Ala Trp Tyr Glu Leu Th
#r Pro Ala Glu Thr Ser
                                1525
# 1530
# 1535
Val Arg Leu Arg Ala Tyr Leu Asn Thr Pro Gl
#y Leu Pro Val Cys Gln
                        1540
# 1545
# 1550
Asp His Leu Glu Phe Trp Glu Ser Val Phe Th
#r Gly Leu Thr His Ile
                1555
# 1560
# 1565
Asp Ala His Phe Leu Ser Gln Thr Lys Gln Al
#a Gly Asp Asn Phe Pro
        1570
# 1575
# 1580
Tyr Leu Val Ala Tyr Gln Ala Thr Val Cys Al
#a Arg Ala Gln Ala Pro
1585 1590
# 1595
# 1600
Pro Pro Ser Trp Asp Gln Met Trp Lys Cys Le
#u Ile Arg Leu Lys Pro
                                1605
# 1610
# 1615
Thr Leu His Gly Pro Thr Pro Leu Leu Tyr Ar
#g Leu Gly Ala Val Gln
                        1620
# 1625
# 1630
Asn Glu Val Thr Thr Thr His Pro Ile Thr Ly
#s Tyr Ile Met Ala Cys
                1635
# 1640
# 1645
Met Ser Ala Asp Leu Glu Val Val Thr Ser Th
#r Trp Val Leu Val Gly
        1650
# 1655
# 1660
Gly Val Leu Ala Ala Leu Ala Ala Tyr Cys Le
#u Thr Thr Gly Ser Val
1665 1670
# 1675
# 1680
Val Ile Val Gly Arg Ile Ile Leu Ser Gly Ly
#s Pro Ala Ile Ile Pro
                                1685
# 1690
# 1695
Asp Arg Glu Val Leu Tyr Arg Glu Phe Asp Gl
#u Met Glu Glu Cys Ala
                        1700
# 1705
# 1710
Ser His Leu Pro Tyr Ile Glu Gln Gly Met Gl
#n Leu Ala Glu Gln Phe
                1715
# 1720
# 1725
Lys Gln Lys Ala Ile Gly Leu Leu Gln Thr Al
#a Thr Lys Gln Ala Glu
        1730
# 1735
# 1740
Ala Ala Ala Pro Val Val Glu Ser Lys Trp Ar
#g Thr Leu Glu Ala Phe
1745 1750
# 1755
# 1760
Trp Ala Lys His Met Trp Asn Phe Ile Ser Gl
#y Ile Gln Tyr Leu Ala
                                1765
# 1770
# 1775
Gly Leu Ser Thr Leu Pro Gly Asn Pro Ala Il
#e Ala Ser Leu Met Ala
                        1780
# 1785
# 1790
Phe Thr Ala Ser Ile Thr Ser Pro Leu Thr Th
#r Gln His Thr Leu Leu
                1795
# 1800
# 1805
Phe Asn Ile Leu Gly Gly Trp Val Ala Ala Gl
#n Leu Ala Pro Pro Ser
        1810
# 1815
# 1820
Ala Ala Ser Ala Phe Val Gly Ala Gly Ile Al
#a Gly Ala Ala Val Gly
1825 1830
# 1835
# 1840
Ser Ile Gly Leu Gly Lys Val Leu Val Asp Il
#e Leu Ala Gly Tyr Gly
                                1845
# 1850
# 1855
Ala Gly Val Ala Gly Ala Leu Val Ala Phe Ly
#s Val Met Ser Gly Glu
                        1860
# 1865
# 1870
Met Pro Ser Thr Glu Asp Leu Val Asn Leu Le
#u Pro Ala Ile Leu Ser
                1875
# 1880
# 1885
Pro Gly Ala Leu Val Val Gly Val Val Cys Al
#a Ala Ile Leu Arg Arg
        1890
# 1895
# 1900
His Val Gly Pro Gly Glu Gly Ala Val Gln Tr
#p Met Asn Arg Leu Ile
1905 1910
# 1915
# 1920
Ala Phe Ala Ser Arg Gly Asn His Val Ser Pr
#o Thr His Tyr Val Pro
                                1925
# 1930
# 1935
Glu Ser Asp Ala Ala Ala Arg Val Thr Gln Il
#e Leu Ser Ser Leu Thr
                        1940
# 1945
# 1950
Ile Thr Gln Leu Leu Lys Arg Leu His Gln Tr
#p Ile Asn Glu Asp Cys
                1955
# 1960
# 1965
Ser Thr Pro Cys Ser Gly Ser Trp Leu Arg As
#p Val Trp Asp Trp Ile
        1970
# 1975
# 1980
Cys Thr Val Leu Thr Asp Phe Lys Thr Trp Le
#u Gln Ser Lys Leu Leu
1985 1990
# 1995
# 2000
Pro Arg Leu Pro Gly Val Pro Phe Phe Ser Cy
#s Gln Arg Gly Tyr Lys
                                2005
# 2010
# 2015
Gly Val Trp Arg Gly Asp Gly Ile Met Gln Th
#r Thr Cys Pro Cys Gly
                        2020
# 2025
# 2030
Ala Gln Ile Thr Gly His Val Lys Asn Gly Se
#r Met Arg Ile Val Gly
                2035
# 2040
# 2045
Pro Arg Thr Cys Ser Asn Thr Trp His Gly Th
#r Phe Pro Ile Asn Ala
        2050
# 2055
# 2060
Tyr Thr Thr Gly Pro Cys Thr Pro Ser Pro Al
#a Pro Asn Tyr Ser Arg
2065 2070
# 2075
# 2080
Ala Leu Trp Arg Val Ala Ala Glu Glu Tyr Va
#l Glu Val Thr Arg Val
                                2085
# 2090
# 2095
Gly Asp Phe His Tyr Val Thr Gly Met Thr Th
#r Asp Asn Val Lys Cys
                        2100
# 2105
# 2110
Pro Cys Gln Val Pro Ala Pro Glu Phe Phe Th
#r Glu Val Asp Gly Val
                2115
# 2120
# 2125
Arg Leu His Arg Tyr Ala Pro Ala Cys Lys Pr
#o Leu Leu Arg Glu Glu
        2130
# 2135
# 2140
Val Thr Phe Leu Val Gly Leu Asn Gln Tyr Le
#u Val Gly Ser Gln Leu
2145 2150
# 2155
# 2160
Pro Cys Glu Pro Glu Pro Asp Val Ala Val Le
#u Thr Ser Met Leu Thr
                                2165
# 2170
# 2175
Asp Pro Ser His Ile Thr Ala Glu Thr Ala Ly
#s Arg Arg Leu Ala Arg
                        2180
# 2185
# 2190
Gly Ser Pro Pro Ser Leu Ala Ser Ser Ser Al
#a Ser Gln Leu Ser Ala
                2195
# 2200
# 2205
Pro Ser Leu Lys Ala Thr Cys Thr Thr Arg Hi
#s Asp Ser Pro Asp Ala
        2210
# 2215
# 2220
Asp Leu Ile Glu Ala Asn Leu Leu Trp Arg Gl
#n Glu Met Gly Gly Asn
2225 2230
# 2235
# 2240
Ile Thr Arg Val Glu Ser Glu Asn Lys Val Va
#l Ile Leu Asp Ser Phe
                                2245
# 2250
# 2255
Glu Pro Leu Gln Ala Glu Glu Asp Glu Arg Gl
#u Val Ser Val Pro Ala
                        2260
# 2265
# 2270
Glu Ile Leu Arg Arg Ser Arg Lys Phe Pro Ar
#g Ala Met Pro Ile Trp
                2275
# 2280
# 2285
Ala Arg Pro Asp Tyr Asn Pro Pro Leu Leu Gl
#u Ser Trp Lys Asp Pro
        2290
# 2295
# 2300
Asp Tyr Val Pro Pro Val Val His Gly Cys Pr
#o Leu Pro Pro Ala Lys
2305 2310
# 2315
# 2320
Ala Pro Pro Ile Pro Pro Pro Arg Arg Lys Ar
#g Thr Val Val Leu Ser
                                2325
# 2330
# 2335
Glu Ser Thr Val Ser Ser Ala Leu Ala Glu Le
#u Ala Thr Lys Thr Phe
                        2340
# 2345
# 2350
Gly Ser Ser Glu Ser Ser Ala Val Asp Ser Gl
#y Thr Ala Thr Ala Ser
                2355
# 2360
# 2365
Pro Asp Gln Pro Ser Asp Asp Gly Asp Ala Gl
#y Ser Asp Val Glu Ser
        2370
# 2375
# 2380
Tyr Ser Ser Met Pro Pro Leu Glu Gly Glu Pr
#o Gly Asp Pro Asp Leu
2385 2390
# 2395
# 2400
Ser Asp Gly Ser Trp Ser Thr Val Ser Glu Gl
#u Ala Ser Glu Asp Val
                                2405
# 2410
# 2415
Val Cys Cys Ser Met Ser Tyr Thr Trp Thr Gl
#y Ala Leu Ile Thr Pro
                        2420
# 2425
# 2430
Cys Ala Ala Glu Glu Thr Lys Leu Pro Ile As
#n Ala Leu Ser Asn Ser
                2435
# 2440
# 2445
Leu Leu Arg His His Asn Leu Val Tyr Ala Th
#r Thr Ser Arg Ser Ala
        2450
# 2455
# 2460
Ser Leu Arg Gln Lys Lys Val Thr Phe Asp Ar
#g Leu Gln Val Leu Asp
2465 2470
# 2475
# 2480
Asp His Tyr Arg Asp Val Leu Lys Glu Met Ly
#s Ala Lys Ala Ser Thr
                                2485
# 2490
# 2495
Val Lys Ala Lys Leu Leu Ser Val Glu Glu Al
#a Cys Lys Leu Thr Pro
                        2500
# 2505
# 2510
Pro His Ser Ala Arg Ser Lys Phe Gly Tyr Gl
#y Ala Lys Asp Val Arg
                2515
# 2520
# 2525
Asn Leu Ser Ser Lys Ala Val Asn His Ile Ar
#g Ser Val Trp Lys Asp
        2530
# 2535
# 2540
Leu Leu Glu Asp Thr Glu Thr Pro Ile Asp Th
#r Thr Ile Met Ala Lys
2545 2550
# 2555
# 2560
Asn Glu Val Phe Cys Val Gln Pro Glu Lys Gl
#y Gly Arg Lys Pro Ala
                                2565
# 2570
# 2575
Arg Leu Ile Val Phe Pro Asp Leu Gly Val Ar
#g Val Cys Glu Lys Met
                        2580
# 2585
# 2590
Ala Leu Tyr Asp Val Val Ser Thr Leu Pro Gl
#n Ala Val Met Gly Ser
                2595
# 2600
# 2605
Ser Tyr Gly Phe Gln Tyr Ser Pro Gly Gln Ar
#g Val Glu Phe Leu Val
        2610
# 2615
# 2620
Asn Ala Trp Lys Ala Lys Lys Cys Pro Met Gl
#y Phe Ala Tyr Asp Thr
2625 2630
# 2635
# 2640
Arg Cys Phe Asp Ser Thr Val Thr Glu Asn As
#p Ile Arg Val Glu Glu
                                2645
# 2650
# 2655
Ser Ile Tyr Gln Cys Cys Asp Leu Ala Pro Gl
#u Ala Arg Gln Ala Ile
                        2660
# 2665
# 2670
Arg Ser Leu Thr Glu Arg Leu Tyr Ile Gly Gl
#y Pro Leu Thr Asn Ser
                2675
# 2680
# 2685
Lys Gly Gln Asn Cys Gly Tyr Arg Arg Cys Ar
#g Ala Ser Gly Val Leu
        2690
# 2695
# 2700
Thr Thr Ser Cys Gly Asn Thr Leu Thr Cys Ty
#r Leu Lys Ala Ala Ala
2705 2710
# 2715
# 2720
Ala Cys Arg Ala Ala Lys Leu Gln Asp Cys Th
#r Met Leu Val Cys Gly
                                2725
# 2730
# 2735
Asp Asp Leu Val Val Ile Cys Glu Ser Ala Gl
#y Thr Gln Glu Asp Glu
                        2740
# 2745
# 2750
Ala Ser Leu Arg Ala Phe Thr Glu Ala Met Th
#r Arg Tyr Ser Ala Pro
                2755
# 2760
# 2765
Pro Gly Asp Pro Pro Lys Pro Glu Tyr Asp Le
#u Glu Leu Ile Thr Ser
        2770
# 2775
# 2780
Cys Ser Ser Asn Val Ser Val Ala His Asp Al
#a Ser Gly Lys Arg Val
2785 2790
# 2795
# 2800
Tyr Tyr Leu Thr Arg Asp Pro Thr Thr Pro Le
#u Ala Arg Ala Ala Trp
                                2805
# 2810
# 2815
Glu Thr Ala Arg His Thr Pro Val Asn Ser Tr
#p Leu Gly Asn Ile Ile
                        2820
# 2825
# 2830
Met Tyr Ala Pro Thr Leu Trp Ala Arg Met Il
#e Leu Met Thr His Phe
                2835
# 2840
# 2845
Phe Ser Ile Leu Leu Ala Gln Glu Gln Leu Gl
#u Lys Ala Leu Asp Cys
        2850
# 2855
# 2860
Gln Ile Tyr Gly Ala Cys Tyr Ser Ile Glu Pr
#o Leu Asp Leu Pro Gln
2865 2870
# 2875
# 2880
Ile Ile Gln Arg Leu His Gly Leu Ser Ala Ph
#e Ser Leu His Ser Tyr
                                2885
# 2890
# 2895
Ser Pro Gly Glu Ile Asn Arg Val Ala Ser Cy
#s Leu Arg Lys Leu Gly
                        2900
# 2905
# 2910
Val Pro Pro Leu Arg Val Trp Arg His Arg Al
#a Arg Ser Val Arg Ala
                2915
# 2920
# 2925
Arg Leu Leu Ser Gln Gly Gly Arg Ala Ala Th
#r Cys Gly Lys Tyr Leu
        2930
# 2935
# 2940
Phe Asn Trp Ala Val Arg Thr Lys Leu Lys Le
#u Thr Pro Ile Pro Ala
2945 2950
# 2955
# 2960
Ala Ser Gln Leu Asp Leu Ser Ser Trp Phe Va
#l Ala Gly Tyr Ser Gly
                                2965
# 2970
# 2975
Gly Asp Ile Tyr His Ser Leu Ser Arg Ala Ar
#g Pro Arg Trp Phe Met
                        2980
# 2985
# 2990
Trp Cys Leu Leu Leu Leu Ser Val Gly Val Gl
#y Ile Tyr Leu Leu Pro
                2995
# 3000
# 3005
Asn Arg
        3010
<210> SEQ ID NO 4
<211> LENGTH: 18
<212> TYPE: PRT
<213> ORGANISM: Hepatitis C Virus
<400> SEQUENCE: 4
Gln Ile Val Gly Gly Val Tyr Leu Leu Pro Ar
#g Arg Gly Pro Arg Leu
  1 5
# 10
# 15
Gly Val
<210> SEQ ID NO 5
<211> LENGTH: 18
<212> TYPE: PRT
<213> ORGANISM: Hepatitis C Virus
<400> SEQUENCE: 5
Gln Pro Gly Tyr Pro Trp Pro Leu Tyr Gly As
#n Glu Gly Cys Gly Trp
  1 5
# 10
# 15
Ala Gly
<210> SEQ ID NO 6
<211> LENGTH: 18
<212> TYPE: PRT
<213> ORGANISM: Hepatitis C Virus
<400> SEQUENCE: 6
Leu Tyr Gly Asn Glu Gly Cys Gly Trp Ala Gl
#y Trp Leu Leu Ser Pro
  1 5
# 10
# 15
Arg Gly
<210> SEQ ID NO 7
<211> LENGTH: 18
<212> TYPE: PRT
<213> ORGANISM: Hepatitis C Virus
<400> SEQUENCE: 7
Gly Trp Ala Gly Trp Leu Leu Ser Pro Arg Gl
#y Ser Arg Pro Ser Trp
  1 5
# 10
# 15
Gly Pro
<210> SEQ ID NO 8
<211> LENGTH: 18
<212> TYPE: PRT
<213> ORGANISM: Hepatitis C Virus
<400> SEQUENCE: 8
Ile Phe Leu Leu Ala Leu Leu Ser Cys Leu Th
#r Val Pro Ala Ser Ala
  1 5
# 10
# 15
Tyr Gln
<210> SEQ ID NO 9
<211> LENGTH: 18
<212> TYPE: PRT
<213> ORGANISM: Hepatitis C Virus
<400> SEQUENCE: 9
Asp Ala Ile Leu His Thr Pro Gly Cys Val Pr
#o Cys Val Arg Glu Gly
  1 5
# 10
# 15
Asn Ala
<210> SEQ ID NO 10
<211> LENGTH: 18
<212> TYPE: PRT
<213> ORGANISM: Hepatitis C Virus
<400> SEQUENCE: 10
Leu Pro Thr Thr Gln Leu Arg Arg His Ile As
#p Leu Leu Val Gly Ser
  1 5
# 10
# 15
Ala Thr
<210> SEQ ID NO 11
<211> LENGTH: 18
<212> TYPE: PRT
<213> ORGANISM: Hepatitis C Virus
<400> SEQUENCE: 11
Arg His Ile Asp Leu Leu Val Gly Ser Ala Th
#r Leu Cys Ser Ala Leu
  1 5
# 10
# 15
Tyr Val
<210> SEQ ID NO 12
<211> LENGTH: 18
<212> TYPE: PRT
<213> ORGANISM: Hepatitis C Virus
<400> SEQUENCE: 12
Gly Ser Ala Thr Leu Cys Ser Ala Leu Tyr Va
#l Gly Asp Leu Cys Gly
  1 5
# 10
# 15
Ser Val
<210> SEQ ID NO 13
<211> LENGTH: 18
<212> TYPE: PRT
<213> ORGANISM: Hepatitis C Virus
<400> SEQUENCE: 13
Ala Leu Tyr Val Gly Asp Leu Cys Gly Ser Va
#l Phe Leu Val Gly Gln
  1 5
# 10
# 15
Leu Phe
<210> SEQ ID NO 14
<211> LENGTH: 18
<212> TYPE: PRT
<213> ORGANISM: Hepatitis C Virus
<400> SEQUENCE: 14
Ile Met Asp Met Ile Ala Gly Ala His Trp Gl
#y Val Leu Ala Gly Ile
  1 5
# 10
# 15
Ala Tyr
<210> SEQ ID NO 15
<211> LENGTH: 18
<212> TYPE: PRT
<213> ORGANISM: Hepatitis C Virus
<400> SEQUENCE: 15
His Ile Asn Ser Thr Ala Leu Asn Cys Asn Gl
#u Ser Leu Asn Thr Gly
  1 5
# 10
# 15
Trp Leu
<210> SEQ ID NO 16
<211> LENGTH: 18
<212> TYPE: PRT
<213> ORGANISM: Hepatitis C Virus
<400> SEQUENCE: 16
Asn Cys Asn Glu Ser Leu Asn Thr Gly Trp Le
#u Ala Gly Leu Phe Tyr
  1 5
# 10
# 15
Gln His
<210> SEQ ID NO 17
<211> LENGTH: 18
<212> TYPE: PRT
<213> ORGANISM: Hepatitis C Virus
<400> SEQUENCE: 17
Leu Ala Ser Cys Arg Arg Leu Thr Asp Phe Al
#a Gln Gly Trp Gly Pro
  1 5
# 10
# 15
Ile Ser
<210> SEQ ID NO 18
<211> LENGTH: 18
<212> TYPE: PRT
<213> ORGANISM: Hepatitis C Virus
<400> SEQUENCE: 18
Thr Asp Phe Ala Gln Gly Trp Gly Pro Ile Se
#r Tyr Ala Asn Gly Ser
  1 5
# 10
# 15
Gly Leu
<210> SEQ ID NO 19
<211> LENGTH: 18
<212> TYPE: PRT
<213> ORGANISM: Hepatitis C Virus
<400> SEQUENCE: 19
Gly Pro Ile Ser Tyr Ala Asn Gly Ser Gly Le
#u Asp Glu Arg Pro Tyr
  1 5
# 10
# 15
Cys Trp
<210> SEQ ID NO 20
<211> LENGTH: 18
<212> TYPE: PRT
<213> ORGANISM: Hepatitis C Virus
<400> SEQUENCE: 20
Gly Ser Gly Leu Asp Glu Arg Pro Tyr Cys Tr
#p His Tyr Pro Pro Arg
  1 5
# 10
# 15
Pro Cys
<210> SEQ ID NO 21
<211> LENGTH: 18
<212> TYPE: PRT
<213> ORGANISM: Hepatitis C Virus
<400> SEQUENCE: 21
Trp Met Asn Ser Thr Gly Phe Thr Lys Val Cy
#s Gly Ala Pro Pro Cys
  1 5
# 10
# 15
Val Ile
<210> SEQ ID NO 22
<211> LENGTH: 18
<212> TYPE: PRT
<213> ORGANISM: Hepatitis C Virus
<400> SEQUENCE: 22
Pro Cys Val Ile Gly Gly Val Gly Asn Asn Th
#r Leu Leu Cys Pro Thr
  1 5
# 10
# 15
Asp Cys
<210> SEQ ID NO 23
<211> LENGTH: 18
<212> TYPE: PRT
<213> ORGANISM: Hepatitis C Virus
<400> SEQUENCE: 23
Met Tyr Val Gly Gly Val Glu His Arg Leu Gl
#u Ala Ala Cys Asn Trp
  1 5
# 10
# 15
Thr Arg
<210> SEQ ID NO 24
<211> LENGTH: 18
<212> TYPE: PRT
<213> ORGANISM: Hepatitis C Virus
<400> SEQUENCE: 24
Tyr Leu Tyr Gly Val Gly Ser Ser Ile Ala Se
#r Trp Ala Ile Lys Trp
  1 5
# 10
# 15
Glu Tyr
<210> SEQ ID NO 25
<211> LENGTH: 18
<212> TYPE: PRT
<213> ORGANISM: Hepatitis C Virus
<400> SEQUENCE: 25
Ser Ile Ala Ser Trp Ala Ile Lys Trp Glu Ty
#r Val Val Leu Leu Phe
  1 5
# 10
# 15
Leu Leu
<210> SEQ ID NO 26
<211> LENGTH: 18
<212> TYPE: PRT
<213> ORGANISM: Hepatitis C Virus
<400> SEQUENCE: 26
Lys Trp Glu Tyr Val Val Leu Leu Phe Leu Le
#u Leu Ala Asp Ala Arg
  1 5
# 10
# 15
Val Cys
<210> SEQ ID NO 27
<211> LENGTH: 18
<212> TYPE: PRT
<213> ORGANISM: Hepatitis C Virus
<400> SEQUENCE: 27
Trp Met Met Leu Leu Ile Ser Gln Ala Glu Al
#a Ala Leu Glu Asn Leu
  1 5
# 10
# 15
Val Ile
<210> SEQ ID NO 28
<211> LENGTH: 18
<212> TYPE: PRT
<213> ORGANISM: Hepatitis C Virus
<400> SEQUENCE: 28
Gly Ala Val Tyr Ala Phe Tyr Gly Met Trp Pr
#o Leu Leu Leu Leu Leu
  1 5
# 10
# 15
Leu Ala
<210> SEQ ID NO 29
<211> LENGTH: 18
<212> TYPE: PRT
<213> ORGANISM: Hepatitis C Virus
<400> SEQUENCE: 29
Gly Met Trp Pro Leu Leu Leu Leu Leu Leu Al
#a Leu Pro Gln Arg Ala
  1 5
# 10
# 15
Tyr Ala
<210> SEQ ID NO 30
<211> LENGTH: 18
<212> TYPE: PRT
<213> ORGANISM: Hepatitis C Virus
<400> SEQUENCE: 30
Thr Leu Val Phe Asp Ile Thr Lys Leu Leu Le
#u Ala Ile Phe Gly Pro
  1 5
# 10
# 15
Leu Trp
<210> SEQ ID NO 31
<211> LENGTH: 14
<212> TYPE: PRT
<213> ORGANISM: Hepatitis C Virus
<400> SEQUENCE: 31
Val Ser Thr Ala Thr Gln Thr Phe Leu Ala Th
#r Cys Ile Asn
  1 5
# 10
<210> SEQ ID NO 32
<211> LENGTH: 18
<212> TYPE: PRT
<213> ORGANISM: Hepatitis C Virus
<400> SEQUENCE: 32
Ala Thr Gln Thr Phe Leu Ala Thr Cys Ile As
#n Gly Val Cys Trp Thr
  1 5
# 10
# 15
Val Tyr
<210> SEQ ID NO 33
<211> LENGTH: 18
<212> TYPE: PRT
<213> ORGANISM: Hepatitis C Virus
<400> SEQUENCE: 33
Asp Ser Ser Val Leu Cys Glu Cys Tyr Asp Al
#a Gly Cys Ala Trp Tyr
  1 5
# 10
# 15
Glu Leu
<210> SEQ ID NO 34
<211> LENGTH: 18
<212> TYPE: PRT
<213> ORGANISM: Hepatitis C Virus
<400> SEQUENCE: 34
Ala Tyr Met Asn Thr Pro Gly Leu Pro Val Cy
#s Gln Asp His Leu Glu
  1 5
# 10
# 15
Phe Trp
<210> SEQ ID NO 35
<211> LENGTH: 18
<212> TYPE: PRT
<213> ORGANISM: Hepatitis C Virus
<400> SEQUENCE: 35
Leu Glu Phe Trp Glu Gly Val Phe Thr Gly Le
#u Thr His Ile Asp Ala
  1 5
# 10
# 15
His Phe
<210> SEQ ID NO 36
<211> LENGTH: 18
<212> TYPE: PRT
<213> ORGANISM: Hepatitis C Virus
<400> SEQUENCE: 36
His Pro Ile Thr Lys Tyr Ile Met Thr Cys Me
#t Ser Ala Asp Leu Glu
  1 5
# 10
# 15
Val Val
<210> SEQ ID NO 37
<211> LENGTH: 15
<212> TYPE: PRT
<213> ORGANISM: Hepatitis C Virus
<400> SEQUENCE: 37
Val Thr Ser Thr Trp Val Leu Val Gly Gly Va
#l Leu Ala Ala Leu
  1 5
# 10
# 15
<210> SEQ ID NO 38
<211> LENGTH: 18
<212> TYPE: PRT
<213> ORGANISM: Hepatitis C Virus
<400> SEQUENCE: 38
Trp Val Leu Val Gly Gly Val Leu Ala Ala Le
#u Ala Ala Tyr Cys Leu
  1 5
# 10
# 15
Ser Thr
<210> SEQ ID NO 39
<211> LENGTH: 15
<212> TYPE: PRT
<213> ORGANISM: Hepatitis C Virus
<400> SEQUENCE: 39
Leu Ala Ala Leu Ala Ala Tyr Cys Leu Ser Th
#r Gly Cys Val Val
  1 5
# 10
# 15
<210> SEQ ID NO 40
<211> LENGTH: 18
<212> TYPE: PRT
<213> ORGANISM: Hepatitis C Virus
<400> SEQUENCE: 40
Glu Val Phe Trp Ala Lys His Met Trp Asn Ph
#e Ile Ser Gly Ile Gln
  1 5
# 10
# 15
Tyr Leu
<210> SEQ ID NO 41
<211> LENGTH: 18
<212> TYPE: PRT
<213> ORGANISM: Hepatitis C Virus
<400> SEQUENCE: 41
Met Trp Asn Phe Ile Ser Gly Ile Gln Tyr Le
#u Ala Gly Leu Ser Thr
  1 5
# 10
# 15
Leu Pro
<210> SEQ ID NO 42
<211> LENGTH: 18
<212> TYPE: PRT
<213> ORGANISM: Hepatitis C Virus
<400> SEQUENCE: 42
Pro Ala Ile Leu Ser Pro Gly Ala Leu Val Va
#l Gly Val Val Cys Ala
  1 5
# 10
# 15
Ala Ile
<210> SEQ ID NO 43
<211> LENGTH: 18
<212> TYPE: PRT
<213> ORGANISM: Hepatitis C Virus
<400> SEQUENCE: 43
Ser Trp Leu Arg Asp Ile Trp Asp Trp Ile Cy
#s Glu Val Leu Ser Asp
  1 5
# 10
# 15
Phe Lys
<210> SEQ ID NO 44
<211> LENGTH: 18
<212> TYPE: PRT
<213> ORGANISM: Hepatitis C Virus
<400> SEQUENCE: 44
Asp Trp Ile Cys Glu Val Leu Ser Asp Phe Ly
#s Thr Trp Leu Lys Ala
  1 5
# 10
# 15
Lys Leu
<210> SEQ ID NO 45
<211> LENGTH: 18
<212> TYPE: PRT
<213> ORGANISM: Hepatitis C Virus
<400> SEQUENCE: 45
Tyr Val Ser Gly Met Thr Thr Asp Asn Leu Ly
#s Cys Pro Cys Gln Ile
  1 5
# 10
# 15
Pro Ser
<210> SEQ ID NO 46
<211> LENGTH: 15
<212> TYPE: PRT
<213> ORGANISM: Hepatitis C Virus
<400> SEQUENCE: 46
Ser Ser Gly Ala Asp Thr Glu Asp Val Val Cy
#s Cys Ser Met Ser
  1 5
# 10
# 15
<210> SEQ ID NO 47
<211> LENGTH: 14
<212> TYPE: PRT
<213> ORGANISM: Hepatitis C Virus
<400> SEQUENCE: 47
Asp Thr Glu Asp Val Val Cys Cys Ser Met Se
#r Tyr Ser Trp
  1 5
# 10
<210> SEQ ID NO 48
<211> LENGTH: 18
<212> TYPE: PRT
<213> ORGANISM: Hepatitis C Virus
<400> SEQUENCE: 48
Ser Ser Gly Ala Asp Thr Glu Asp Val Val Cy
#s Cys Ser Met Ser Tyr
  1 5
# 10
# 15
Ser Trp
<210> SEQ ID NO 49
<211> LENGTH: 15
<212> TYPE: PRT
<213> ORGANISM: Hepatitis C Virus
<400> SEQUENCE: 49
Asp Val Val Cys Cys Ser Met Ser Tyr Ser Tr
#p Thr Gly Ala Leu
  1 5
# 10
# 15
<210> SEQ ID NO 50
<211> LENGTH: 18
<212> TYPE: PRT
<213> ORGANISM: Hepatitis C Virus
<400> SEQUENCE: 50
Thr Val Thr Glu Ser Asp Ile Arg Thr Glu Gl
#u Ala Ile Tyr Gln Cys
  1 5
# 10
# 15
Cys Asp
<210> SEQ ID NO 51
<211> LENGTH: 18
<212> TYPE: PRT
<213> ORGANISM: Hepatitis C Virus
<400> SEQUENCE: 51
Gly Asn Thr Leu Thr Cys Tyr Ile Lys Ala Ar
#g Ala Ala Cys Arg Ala
  1 5
# 10
# 15
Ala Gly
<210> SEQ ID NO 52
<211> LENGTH: 18
<212> TYPE: PRT
<213> ORGANISM: Hepatitis C Virus
<400> SEQUENCE: 52
Arg Ala Ala Gly Leu Gln Asp Cys Thr Met Le
#u Val Cys Gly Asp Asp
  1 5
# 10
# 15
Leu Val
<210> SEQ ID NO 53
<211> LENGTH: 18
<212> TYPE: PRT
<213> ORGANISM: Hepatitis C Virus
<400> SEQUENCE: 53
Cys Thr Met Leu Val Cys Gly Asp Asp Leu Va
#l Val Ile Cys Glu Ser
  1 5
# 10
# 15
Ala Gly
<210> SEQ ID NO 54
<211> LENGTH: 18
<212> TYPE: PRT
<213> ORGANISM: Hepatitis C Virus
<400> SEQUENCE: 54
Asp Asp Leu Val Val Ile Cys Glu Ser Ala Gl
#y Val Gln Glu Asp Ala
  1 5
# 10
# 15
Ala Ser
<210> SEQ ID NO 55
<211> LENGTH: 18
<212> TYPE: PRT
<213> ORGANISM: Hepatitis C Virus
<400> SEQUENCE: 55
Leu Glu Leu Ile Thr Ser Cys Ser Ser Asn Va
#l Ser Val Ala His Asp
  1 5
# 10
# 15
Gly Ala
<210> SEQ ID NO 56
<211> LENGTH: 18
<212> TYPE: PRT
<213> ORGANISM: Hepatitis C Virus
<400> SEQUENCE: 56
His Thr Pro Val Asn Ser Trp Leu Gly Asn Il
#e Ile Met Phe Ala Pro
  1 5
# 10
# 15
Thr Leu
<210> SEQ ID NO 57
<211> LENGTH: 18
<212> TYPE: PRT
<213> ORGANISM: Hepatitis C Virus
<400> SEQUENCE: 57
Ala Pro Thr Leu Trp Ala Arg Met Ile Leu Me
#t Thr His Phe Phe Ser
  1 5
# 10
# 15
Val Leu
<210> SEQ ID NO 58
<211> LENGTH: 18
<212> TYPE: PRT
<213> ORGANISM: Hepatitis C Virus
<400> SEQUENCE: 58
Asp Gln Leu Glu Gln Ala Leu Asn Cys Glu Il
#e Tyr Gly Ala Cys Tyr
  1 5
# 10
# 15
Ser Ile
<210> SEQ ID NO 59
<211> LENGTH: 18
<212> TYPE: PRT
<213> ORGANISM: Hepatitis C Virus
<400> SEQUENCE: 59
Gly Val Pro Pro Leu Arg Ala Trp Arg His Ar
#g Ala Arg Ser Val Arg
  1 5
# 10
# 15
Ala Arg
<210> SEQ ID NO 60
<211> LENGTH: 18
<212> TYPE: PRT
<213> ORGANISM: Hepatitis C Virus
<400> SEQUENCE: 60
Trp Arg His Arg Ala Arg Ser Val Arg Ala Ar
#g Leu Leu Ser Arg Gly
  1 5
# 10
# 15
Gly Arg
<210> SEQ ID NO 61
<211> LENGTH: 18
<212> TYPE: PRT
<213> ORGANISM: Hepatitis C Virus
<400> SEQUENCE: 61
Gly Trp Phe Thr Ala Gly Tyr Ser Gly Gly As
#p Ile Tyr His Ser Val
  1 5
# 10
# 15
Ser His
<210> SEQ ID NO 62
<211> LENGTH: 18
<212> TYPE: PRT
<213> ORGANISM: Hepatitis C Virus
<400> SEQUENCE: 62
Leu Tyr Gly Asn Glu Gly Leu Gly Trp Ala Gl
#y Trp Leu Leu Ser Pro
  1 5
# 10
# 15
Arg Gly
<210> SEQ ID NO 63
<211> LENGTH: 18
<212> TYPE: PRT
<213> ORGANISM: Hepatitis C Virus
<400> SEQUENCE: 63
Ile Phe Leu Leu Ala Leu Leu Ser Cys Ile Th
#r Val Pro Val Ser Ala
  1 5
# 10
# 15
Ala Gln
<210> SEQ ID NO 64
<211> LENGTH: 18
<212> TYPE: PRT
<213> ORGANISM: Hepatitis C Virus
<400> SEQUENCE: 64
Ile Phe Leu Leu Ala Leu Leu Ser Cys Leu Th
#r Ile Pro Ala Ser Ala
  1 5
# 10
# 15
Tyr Glu
<210> SEQ ID NO 65
<211> LENGTH: 18
<212> TYPE: PRT
<213> ORGANISM: Hepatitis C Virus
<400> SEQUENCE: 65
Met Ser Ala Thr Phe Cys Ser Ala Leu Tyr Va
#l Gly Asp Leu Cys Gly
  1 5
# 10
# 15
Gly Val
<210> SEQ ID NO 66
<211> LENGTH: 18
<212> TYPE: PRT
<213> ORGANISM: Hepatitis C Virus
<400> SEQUENCE: 66
Gly Ala Ala Ala Leu Cys Ser Ala Met Tyr Va
#l Gly Asp Leu Cys Gly
  1 5
# 10
# 15
Ser Val
<210> SEQ ID NO 67
<211> LENGTH: 18
<212> TYPE: PRT
<213> ORGANISM: Hepatitis C Virus
<400> SEQUENCE: 67
Ala Leu Tyr Val Gly Asp Leu Cys Gly Gly Va
#l Met Leu Ala Ala Gln
  1 5
# 10
# 15
Val Phe
<210> SEQ ID NO 68
<211> LENGTH: 18
<212> TYPE: PRT
<213> ORGANISM: Hepatitis C Virus
<400> SEQUENCE: 68
Ala Met Tyr Val Gly Asp Leu Cys Gly Ser Va
#l Phe Leu Val Ala Gln
  1 5
# 10
# 15
Leu Phe
<210> SEQ ID NO 69
<211> LENGTH: 18
<212> TYPE: PRT
<213> ORGANISM: Hepatitis C Virus
<400> SEQUENCE: 69
Ile Ile Asp Ile Val Ser Gly Ala His Trp Gl
#y Val Met Phe Gly Leu
  1 5
# 10
# 15
Ala Tyr
<210> SEQ ID NO 70
<211> LENGTH: 18
<212> TYPE: PRT
<213> ORGANISM: Hepatitis C Virus
<400> SEQUENCE: 70
Val Val Asp Met Val Ala Gly Ala His Trp Gl
#y Val Leu Ala Gly Leu
  1 5
# 10
# 15
Ala Tyr
<210> SEQ ID NO 71
<211> LENGTH: 18
<212> TYPE: PRT
<213> ORGANISM: Hepatitis C Virus
<400> SEQUENCE: 71
Val Asp Val Gln Tyr Met Tyr Gly Leu Ser Pr
#o Ala Ile Thr Lys Tyr
  1 5
# 10
# 15
Val Val
<210> SEQ ID NO 72
<211> LENGTH: 18
<212> TYPE: PRT
<213> ORGANISM: Hepatitis C Virus
<400> SEQUENCE: 72
Tyr Leu Tyr Gly Ile Gly Ser Ala Val Val Se
#r Phe Ala Ile Lys Trp
  1 5
# 10
# 15
Glu Tyr
<210> SEQ ID NO 73
<211> LENGTH: 18
<212> TYPE: PRT
<213> ORGANISM: Hepatitis C Virus
<400> SEQUENCE: 73
Trp Met Leu Ile Leu Leu Gly Gln Ala Glu Al
#a Ala Leu Glu Lys Leu
  1 5
# 10
# 15
Val Val
<210> SEQ ID NO 74
<211> LENGTH: 18
<212> TYPE: PRT
<213> ORGANISM: Hepatitis C Virus
<400> SEQUENCE: 74
Trp Met Met Leu Leu Ile Ala Gln Ala Glu Al
#a Ala Leu Glu Asn Leu
  1 5
# 10
# 15
Val Val
<210> SEQ ID NO 75
<211> LENGTH: 18
<212> TYPE: PRT
<213> ORGANISM: Hepatitis C Virus
<400> SEQUENCE: 75
Gly Val Val Phe Asp Ile Thr Lys Trp Leu Le
#u Ala Leu Leu Gly Pro
  1 5
# 10
# 15
Ala Tyr
<210> SEQ ID NO 76
<211> LENGTH: 18
<212> TYPE: PRT
<213> ORGANISM: Hepatitis C Virus
<400> SEQUENCE: 76
Glu Leu Ile Phe Thr Ile Thr Lys Ile Leu Le
#u Ala Ile Leu Gly Pro
  1 5
# 10
# 15
Leu Met
<210> SEQ ID NO 77
<211> LENGTH: 18
<212> TYPE: PRT
<213> ORGANISM: Hepatitis C Virus
<400> SEQUENCE: 77
Val Ser Gln Ser Phe Leu Gly Thr Thr Ile Se
#r Gly Val Leu Trp Thr
  1 5
# 10
# 15
Val Tyr
<210> SEQ ID NO 78
<211> LENGTH: 18
<212> TYPE: PRT
<213> ORGANISM: Hepatitis C Virus
<400> SEQUENCE: 78
Ala Thr Gln Ser Phe Leu Ala Thr Cys Val As
#n Gly Val Cys Trp Thr
  1 5
# 10
# 15
Val Tyr
<210> SEQ ID NO 79
<211> LENGTH: 18
<212> TYPE: PRT
<213> ORGANISM: Hepatitis C Virus
<400> SEQUENCE: 79
Ser Trp Leu Arg Asp Val Trp Asp Trp Val Cy
#s Thr Ile Leu Thr Asp
  1 5
# 10
# 15
Phe Lys
<210> SEQ ID NO 80
<211> LENGTH: 18
<212> TYPE: PRT
<213> ORGANISM: Hepatitis C Virus
<400> SEQUENCE: 80
Ser Trp Leu Arg Asp Val Trp Asp Trp Ile Cy
#s Thr Val Leu Thr Asp
  1 5
# 10
# 15
Phe Lys
<210> SEQ ID NO 81
<211> LENGTH: 18
<212> TYPE: PRT
<213> ORGANISM: Hepatitis C Virus
<400> SEQUENCE: 81
Asp Trp Val Cys Thr Ile Leu Thr Asp Phe Ly
#s Asn Trp Leu Thr Ser
  1 5
# 10
# 15
Lys Leu
<210> SEQ ID NO 82
<211> LENGTH: 18
<212> TYPE: PRT
<213> ORGANISM: Hepatitis C Virus
<400> SEQUENCE: 82
Asp Trp Ile Cys Thr Val Leu Thr Asp Phe Ly
#s Thr Trp Leu Gln Ser
  1 5
# 10
# 15
Lys Leu
<210> SEQ ID NO 83
<211> LENGTH: 15
<212> TYPE: PRT
<213> ORGANISM: Hepatitis C Virus
<400> SEQUENCE: 83
Ala Ser Glu Asp Val Tyr Cys Cys Ser Met Se
#r Tyr Thr Trp Thr
  1 5
# 10
# 15
<210> SEQ ID NO 84
<211> LENGTH: 14
<212> TYPE: PRT
<213> ORGANISM: Hepatitis C Virus
<400> SEQUENCE: 84
Glu Asp Asp Thr Thr Val Cys Cys Ser Met Se
#r Tyr Ser Trp
  1 5
# 10
<210> SEQ ID NO 85
<211> LENGTH: 18
<212> TYPE: PRT
<213> ORGANISM: Hepatitis C Virus
<400> SEQUENCE: 85
Cys Thr Met Leu Val Cys Gly Asp Asp Leu Va
#l Val Ile Cys Glu Ser
  1 5
# 10
# 15
Ala Gly
<210> SEQ ID NO 86
<211> LENGTH: 18
<212> TYPE: PRT
<213> ORGANISM: Hepatitis C Virus
<400> SEQUENCE: 86
Pro Thr Met Leu Val Cys Gly Asp Asp Leu Va
#l Val Ile Ser Glu Ser
  1 5
# 10
# 15
Gln Gly
<210> SEQ ID NO 87
<211> LENGTH: 19
<212> TYPE: DNA
<213> ORGANISM: Homo sapiens
<400> SEQUENCE: 87
gaaggtgaag gtcggagtc
#
#
# 19
<210> SEQ ID NO 88
<211> LENGTH: 20
<212> TYPE: DNA
<213> ORGANISM: Homo sapiens
<400> SEQUENCE: 88
gaagatggtg atgggatttc
#
#
# 20
<210> SEQ ID NO 89
<211> LENGTH: 19
<212> TYPE: DNA
<213> ORGANISM: Hepatitis C Virus
<400> SEQUENCE: 89
tctgcggaac cggtgagta
#
#
# 19
<210> SEQ ID NO 90
<211> LENGTH: 19
<212> TYPE: DNA
<213> ORGANISM: Hepatitis C Virus
<400> SEQUENCE: 90
tcaggcagta ccacaaggc
#
#
# 19
<210> SEQ ID NO 91
<211> LENGTH: 18
<212> TYPE: PRT
<213> ORGANISM: Artificial Sequence
<220> FEATURE:
<223> OTHER INFORMATION: A synthetic peptide
<400> SEQUENCE: 91
Ser Trp Leu Arg Pro Ile Trp Pro Trp Ile Cy
#s Glu Val Leu Ser Asp
  1 5
# 10
# 15
Phe Lys
<210> SEQ ID NO 92
<211> LENGTH: 14
<212> TYPE: PRT
<213> ORGANISM: Hepatitis C Virus
<400> SEQUENCE: 92
Ser Trp Leu Arg Asp Ile Trp Asp Trp Ile Cy
#s Glu Val Leu
  1 5
# 10
<210> SEQ ID NO 93
<211> LENGTH: 15
<212> TYPE: PRT
<213> ORGANISM: Hepatitis C Virus
<400> SEQUENCE: 93
Ser Trp Leu Arg Asp Ile Trp Asp Trp Ile Cy
#s Glu Val Leu Ser
  1 5
# 10
# 15
<210> SEQ ID NO 94
<211> LENGTH: 16
<212> TYPE: PRT
<213> ORGANISM: Hepatitis C Virus
<400> SEQUENCE: 94
Ser Trp Leu Arg Asp Ile Trp Asp Trp Ile Cy
#s Glu Val Leu Ser Asp
  1 5
# 10
# 15
<210> SEQ ID NO 95
<211> LENGTH: 17
<212> TYPE: PRT
<213> ORGANISM: Hepatitis C Virus
<400> SEQUENCE: 95
Ser Trp Leu Arg Asp Ile Trp Asp Trp Ile Cy
#s Glu Val Leu Ser Asp
  1 5
# 10
# 15
Phe
<210> SEQ ID NO 96
<211> LENGTH: 18
<212> TYPE: PRT
<213> ORGANISM: Artificial Sequence
<220> FEATURE:
<223> OTHER INFORMATION: A synthetic peptide
<400> SEQUENCE: 96
Lys Phe Asp Ser Leu Val Glu Cys Ile Trp As
#p Trp Ile Asp Arg Leu
  1 5
# 10
# 15
Trp Ser
<210> SEQ ID NO 97
<211> LENGTH: 18
<212> TYPE: PRT
<213> ORGANISM: Artificial Sequence
<220> FEATURE:
<223> OTHER INFORMATION: A synthetic peptide
<400> SEQUENCE: 97
Ser Ile Trp Arg Asp Trp Val Asp Leu Ile Cy
#s Glu Phe Leu Ser Asp
  1 5
# 10
# 15
Trp Lys
<210> SEQ ID NO 98
<211> LENGTH: 18
<212> TYPE: PRT
<213> ORGANISM: Artificial Sequence
<220> FEATURE:
<223> OTHER INFORMATION: A synthetic peptide
<400> SEQUENCE: 98
Lys Trp Leu Cys Arg Ile Trp Ser Trp Ile Se
#r Asp Val Leu Asp Asp
  1 5
# 10
# 15
Phe Glu
<210> SEQ ID NO 99
<211> LENGTH: 17
<212> TYPE: PRT
<213> ORGANISM: Artificial Sequence
<220> FEATURE:
<223> OTHER INFORMATION: A synthetic peptide
<400> SEQUENCE: 99
Phe Asp Ser Leu Val Glu Cys Ile Trp Asp Tr
#p Ile Asp Arg Leu Trp
  1 5
# 10
# 15
Ser
<210> SEQ ID NO 100
<211> LENGTH: 16
<212> TYPE: PRT
<213> ORGANISM: Artificial Sequence
<220> FEATURE:
<223> OTHER INFORMATION: A synthetic peptide
<400> SEQUENCE: 100
Asp Ser Leu Val Glu Cys Ile Trp Asp Trp Il
#e Asp Arg Leu Trp Ser
  1 5
# 10
# 15
<210> SEQ ID NO 101
<211> LENGTH: 15
<212> TYPE: PRT
<213> ORGANISM: Artificial Sequence
<220> FEATURE:
<223> OTHER INFORMATION: A synthetic peptide
<400> SEQUENCE: 101
Ser Leu Val Glu Cys Ile Trp Asp Trp Ile As
#p Arg Leu Trp Ser
  1 5
# 10
# 15
<210> SEQ ID NO 102
<211> LENGTH: 14
<212> TYPE: PRT
<213> ORGANISM: Artificial Sequence
<220> FEATURE:
<223> OTHER INFORMATION: A synthetic peptide
<400> SEQUENCE: 102
Leu Val Glu Cys Ile Trp Asp Trp Ile Asp Ar
#g Leu Trp Ser
  1 5
# 10
<210> SEQ ID NO 103
<211> LENGTH: 13
<212> TYPE: PRT
<213> ORGANISM: Hepatitis C Virus
<400> SEQUENCE: 103
Ser Trp Leu Arg Asp Ile Trp Asp Trp Ile Cy
#s Glu Val
  1 5
# 10
<210> SEQ ID NO 104
<211> LENGTH: 12
<212> TYPE: PRT
<213> ORGANISM: Hepatitis C Virus
<400> SEQUENCE: 104
Ser Trp Leu Arg Asp Ile Trp Asp Trp Ile Cy
#s Glu
  1 5
# 10
<210> SEQ ID NO 105
<211> LENGTH: 10
<212> TYPE: PRT
<213> ORGANISM: Hepatitis C Virus
<400> SEQUENCE: 105
Ser Trp Leu Arg Asp Ile Trp Asp Trp Ile
  1 5
# 10
<210> SEQ ID NO 106
<211> LENGTH: 8
<212> TYPE: PRT
<213> ORGANISM: Hepatitis C Virus
<400> SEQUENCE: 106
Ser Trp Leu Arg Asp Ile Trp Asp
  1 5
<210> SEQ ID NO 107
<211> LENGTH: 16
<212> TYPE: PRT
<213> ORGANISM: Hepatitis C Virus
<400> SEQUENCE: 107
Leu Arg Asp Ile Trp Asp Trp Ile Cys Glu Va
#l Leu Ser Asp Phe Lys
  1 5
# 10
# 15
<210> SEQ ID NO 108
<211> LENGTH: 14
<212> TYPE: PRT
<213> ORGANISM: Hepatitis C Virus
<400> SEQUENCE: 108
Asp Ile Trp Asp Trp Ile Cys Glu Val Leu Se
#r Asp Phe Lys
  1 5
# 10
<210> SEQ ID NO 109
<211> LENGTH: 12
<212> TYPE: PRT
<213> ORGANISM: Hepatitis C Virus
<400> SEQUENCE: 109
Trp Asp Trp Ile Cys Glu Val Leu Ser Asp Ph
#e Lys
  1 5
# 10
<210> SEQ ID NO 110
<211> LENGTH: 10
<212> TYPE: PRT
<213> ORGANISM: Hepatitis C Virus
<400> SEQUENCE: 110
Trp Ile Cys Glu Val Leu Ser Asp Phe Lys
  1 5
# 10
<210> SEQ ID NO 111
<211> LENGTH: 8
<212> TYPE: PRT
<213> ORGANISM: Hepatitis C Virus
<400> SEQUENCE: 111
Cys Glu Val Leu Ser Asp Phe Lys
  1 5
<210> SEQ ID NO 112
<211> LENGTH: 14
<212> TYPE: PRT
<213> ORGANISM: Artificial Sequence
<220> FEATURE:
<221> NAME/KEY: VARIANT
<222> LOCATION: 1, 4, 5, 8, 11, 12
<223> OTHER INFORMATION: Xaa = any polar ami
#no acid
<220> FEATURE:
<221> NAME/KEY: VARIANT
<222> LOCATION: 2, 3, 6, 7, 9, 10, 13, 14
<223> OTHER INFORMATION: Xaa= any nonpolar amin
#o acid
<220> FEATURE:
<223> OTHER INFORMATION: A synthetic peptide
<400> SEQUENCE: 112
Xaa Xaa Xaa Xaa Xaa Xaa Xaa Xaa Xaa Xaa Xa
#a Xaa Xaa Xaa
  1 5
# 10
<210> SEQ ID NO 113
<211> LENGTH: 15
<212> TYPE: PRT
<213> ORGANISM: Artificial Sequence
<220> FEATURE:
<221> NAME/KEY: VARIANT
<222> LOCATION: 1, 4, 5, 8, 11, 12, 15
<223> OTHER INFORMATION: Xaa = any polar ami
#no acid
<220> FEATURE:
<221> NAME/KEY: VARIANT
<222> LOCATION: 2, 3, 6, 7, 9, 10, 13, 14
<223> OTHER INFORMATION: Xaa = any nonpolar
#amino acid
<220> FEATURE:
<223> OTHER INFORMATION: A synthetic peptide
<400> SEQUENCE: 113
Xaa Xaa Xaa Xaa Xaa Xaa Xaa Xaa Xaa Xaa Xa
#a Xaa Xaa Xaa Xaa
  1 5
# 10
# 15
<210> SEQ ID NO 114
<211> LENGTH: 16
<212> TYPE: PRT
<213> ORGANISM: Artificial Sequence
<220> FEATURE:
<221> NAME/KEY: VARIANT
<222> LOCATION: 1, 4, 5, 8, 11, 12, 15, 1
#6
<223> OTHER INFORMATION: Xaa = any polar ami
#no acid
<220> FEATURE:
<221> NAME/KEY: VARIANT
<222> LOCATION: 2, 3, 6, 7, 9, 10, 13, 14
<223> OTHER INFORMATION: Xaa = any nonpolar
#amino acid
<220> FEATURE:
<223> OTHER INFORMATION: A synthetic peptide
<400> SEQUENCE: 114
Xaa Xaa Xaa Xaa Xaa Xaa Xaa Xaa Xaa Xaa Xa
#a Xaa Xaa Xaa Xaa Xaa
  1 5
# 10
# 15
<210> SEQ ID NO 115
<211> LENGTH: 17
<212> TYPE: PRT
<213> ORGANISM: Artificial Sequence
<220> FEATURE:
<221> NAME/KEY: VARIANT
<222> LOCATION: 1, 4, 5, 8, 11, 12, 15, 1
#6
<223> OTHER INFORMATION: Xaa = any polar ami
#no acid
<220> FEATURE:
<221> NAME/KEY: VARIANT
<222> LOCATION: 2, 3, 6, 7, 9, 10, 13, 14
#, 17
<223> OTHER INFORMATION: Xaa = any nonpolar
#amino acid
<220> FEATURE:
<223> OTHER INFORMATION: A synthetic peptide
<400> SEQUENCE: 115
Xaa Xaa Xaa Xaa Xaa Xaa Xaa Xaa Xaa Xaa Xa
#a Xaa Xaa Xaa Xaa Xaa
  1 5
# 10
# 15
Xaa
<210> SEQ ID NO 116
<211> LENGTH: 18
<212> TYPE: PRT
<213> ORGANISM: Artificial Sequence
<220> FEATURE:
<221> NAME/KEY: VARIANT
<222> LOCATION: 1, 4, 5, 8, 11, 12, 15, 1
#6, 18
<223> OTHER INFORMATION: Xaa = any polar ami
#no acid
<220> FEATURE:
<221> NAME/KEY: VARIANT
<222> LOCATION: 2, 3, 6, 7, 9, 10, 13, 14
#, 17
<223> OTHER INFORMATION: Xaa = any nonpolar
#amino acid
<220> FEATURE:
<223> OTHER INFORMATION: A synthetic peptide
<400> SEQUENCE: 116
Xaa Xaa Xaa Xaa Xaa Xaa Xaa Xaa Xaa Xaa Xa
#a Xaa Xaa Xaa Xaa Xaa
  1 5
# 10
# 15
Xaa Xaa
<210> SEQ ID NO 117
<211> LENGTH: 14
<212> TYPE: PRT
<213> ORGANISM: Artificial Sequence
<220> FEATURE:
<221> NAME/KEY: VARIANT
<222> LOCATION: 1, 4, 7, 8, 11, 12, 14
<223> OTHER INFORMATION: Xaa = any polar ami
#no acid
<220> FEATURE:
<221> NAME/KEY: VARIANT
<222> LOCATION: 2, 3, 5, 6, 9, 10, 13
<223> OTHER INFORMATION: Xaa = any nonpolar
#amino acid
<220> FEATURE:
<223> OTHER INFORMATION: A synthetic peptide
<400> SEQUENCE: 117
Xaa Xaa Xaa Xaa Xaa Xaa Xaa Xaa Xaa Xaa Xa
#a Xaa Xaa Xaa
  1 5
# 10
<210> SEQ ID NO 118
<211> LENGTH: 30
<212> TYPE: PRT
<213> ORGANISM: Artificial Sequence
<220> FEATURE:
<221> NAME/KEY: VARIANT
<222> LOCATION: 1,4,5,8,11,12,15,16,18,19,22,23,26,29,30
<223> OTHER INFORMATION: Xaa = any polar ami
#no acid
<220> FEATURE:
<221> NAME/KEY: VARIANT
<222> LOCATION: 2,3,6,7,9,10,13,14,17,20,21,24,25,27,28
<223> OTHER INFORMATION: Xaa = any nonpolar
#amino acid
<220> FEATURE:
<223> OTHER INFORMATION: A synthetic peptide
<400> SEQUENCE: 118
Xaa Xaa Xaa Xaa Xaa Xaa Xaa Xaa Xaa Xaa Xa
#a Xaa Xaa Xaa Xaa Xaa
  1 5
# 10
# 15
Xaa Xaa Xaa Xaa Xaa Xaa Xaa Xaa Xaa Xaa Xa
#a Xaa Xaa Xaa
                        20
# 25
# 30
```

What is claimed is:

1. An isolated peptide of 14 to 50 D- or L-amino acids in-length, wherein the peptide has an amphipathic α-helical structure, and wherein the peptide has anti-viral activity against a virus of the Flaviviridae family.

2. The peptide of claim 1, with a sequence comprising any one of formulae I-V:
I

| | |
|---|---|
| Xaa₁-Xaa₂-Xaa₃-Xaa₄-Xaa₅-Xaa₆- | (SEQ ID NO: 112) |
| Xaa₇-Xaa₈-Xaa₉-Xaa₁₀-Xaa₁₁-Xaa₁₂- | |
| Xaa₁₃-Xaa₁₄ | |
| II | |
| Xaa₁-Xaa₂-Xaa₃-Xaa₄-Xaa₅-Xaa₆- | (SEQ ID NO: 113) |
| Xaa₇-Xaa₈-Xaa₉-Xaa₁₀-Xaa₁₁-Xaa₁₂- | |
| Xaa₁₃-Xaa₁₄-Xaa₁₅ | |
| III | |
| Xaa₁-Xaa₂-Xaa₃-Xaa₄-Xaa₅-Xaa₆- | (SEQ ID NO: 114) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆ | |
| IV | |
| Xaa₁-Xaa₂-Xaa₃-Xaa₄-Xaa₅-Xaa₆- | (SEQ ID NO: 115) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇ | |
| V | |
| Xaa₁-Xaa₂-Xaa₃-Xaa₄-Xaa₅-Xaa₆- | (SEQ ID NO: 116) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇- | |
| Xaa₁₈ | |

wherein:
Xaa₁, Xaa₄, Xaa₅, Xaa₈, Xaa₁₁, Xaa₁₂, Xaa₁₅, Xaa₁₆ and Xaa₁₈ are separately each a polar amino acid; and
Xaa₂, Xaa₃, Xaa₆, Xaa₇, Xaa₉, Xaa₁₀, Xaa₁₃, Xaa₁₄, and Xaa₁₇ are separately each a nonpolar amino acid.

3. The peptide of claim 2, wherein the nonpolar amino acids are selected from the group consisting of alanine, valine, leucine, methionine, isoleucine, phenylalanine, and tryptophan.

4. The peptide of claim 2, wherein the nonpolar amino acids are selected from the group consisting of valine, leucine, isoleucine, phenylalanine and tryptophan.

5. The peptide of claim 2, wherein the polar amino acids are selected from the group consisting of arginine, asparagine, aspartic acid, cysteine, glutamic acid, glutamine, histidine, homocysteine, lysine, hydroxylysine, ornithine, serine and threonine.

6. The peptide of claim 2, wherein the polar amino acids are selected from the group consisting of arginine, aspartic acid, glutamic acid, cysteine and lysine.

7. The peptide of claim 2, further comprising a 14 amino acid peptide sequence attached by a peptide bond to the N-terminus of a peptide of any of formulae I to V, wherein the 14 amino acid peptide sequence has the structure:

| | |
|---|---|
| Rx-Ry-Ry-Rx-Ry-Ry-Rx-Rx-Ry-Ry-Rx- | (SEQ ID NO: 117) |
| Rx-Ry-Rx | |

wherein each Rx is separately a polar amino acid; and
each Ry is separately a nonpolar amino acid.

8. A peptide comprising at least 14 contiguous amino acids of the peptide of claim 7.

9. The peptide of claim 2, further comprising a twelve amino acid sequence attached by a peptide bond to the carboxy-terminus of formula V, the resulting peptide having the structure
VI

| | |
|---|---|
| Xaa₁-Xaa₂-Xaa₃-Xaa₄-Xaa₅-Xaa₆- | (SEQ ID NO: 118) |
| Xaa₇-Xaa₈-Xaa₉-Xaa₁₀-Xaa₁₁-Xaa₁₂- | |
| Xaa₁₃-Xaa₁₄-Xaa₁₅-Xaa₁₆-Xaa₁₇- | |
| Xaa₁₈-Xaa₁₉-Xaa₂₀-Xaa₂₁-Xaa₂₂- | |
| Xaa₂₃-Xaa₂₄-Xaa₂₅-Xaa₂₆-Xaa₂₇- | |
| Xaa₂₈-Xaa₂₉-Xaa₃₀, | |

wherein
Xaa₁, Xaa₄, Xaa₅, Xaa₈, Xaa₁₁, Xaa₁₂, Xaa₁₅, Xaa₁₆, Xaa₁₈, Xaa₁₉, Xaa₂₂Xaa₂₃, Xaa₂₆, Xaa₂₉, and Xaa₃₀ are separately each a polar amino acid; and
Xaa₂, Xaa₃, Xaa₆, Xaa₇, Xaa₉, Xaa₁₀, Xaa₁₃, Xaa₁₄, Xaa₁₇, Xaa₂₀, Xaa₂₁ Xaa₂₄, Xaa₂₅, Xaa₂₇, and Xaa₂₈ are separately each a nonpolar amino acid.

10. A peptide comprising at least 14 contiguous amino acids of the peptide of claim 9.

11. The peptide of claim 9, further comprising a 14 amino acid peptide sequence attached by a peptide bond to the N-terminus of a peptide of formula VI, wherein the 14 amino acid peptide sequence has the structure:

| | |
|---|---|
| Rx-Ry-Ry-Rx-Ry-Ry-Rx-Rx-Ry-Ry-Rx- | (SEQ ID NO: 117) |
| Rx-Ry-Rx | |

wherein each Rx is separately a polar amino acid; and
each Ry is separately a nonpolar amino acid.

12. A peptide comprising at least 14 contiguous amino acids of the peptide of claim 11.

13. The peptide of claim 1, which has an amino acid composition that consists of arginine, cysteine, glutamate, serine, valine, two aspartates, two leucines, two isoleucines and three tryptophan residues.

14. The peptide of claim 13, which has an amino acid sequence of SEQ ID NO: 92 or 102.

15. The peptide of claim 1, which has an amino acid composition that consists of arginine, cysteine, glutamate, two serines, valine, two aspartates, two leucines, two isoleucines and three tryptophan residues.

16. The peptide of claim 15, which has an amino acid sequence of SEQ ID NO: 93 or 101.

17. The peptide of claim 1, which has an amino acid composition that consists of arginine, cysteine, glutamate, two serines, valine, three aspartates, two leucines, two isoleucines and three tryptophan residues.

18. The peptide of claim 17, which has an amino acid sequence of SEQ ID NO: 94 or 100.

19. The peptide of claim 1, which has an amino acid composition that consists of the residues arginine, cysteine, glutamate, two serines, valine, three aspartates, two leucines, two isoleucines, three tryptophan and a phenylalamine.

20. The peptide of claim 19, which has an amino acid sequence of SEQ ID NO: 95 or 99.

21. The peptide of claim 1, which has an amino acid composition that consists of the residues arginine, cysteine, glutamate, two serines, valine, three aspartates, two leucines, two isoleucines, three tryptophan, a phenylalamine and a lysine.

22. The peptide of claim 21, which has an amino acid sequence of SEQ ID NO: 43 and 96-98.

23. The peptide of claim 22, wherein the EC₅₀ is about 500 nM or less.

24. The peptide of claim 22, wherein the EC₅₀ is about 400 nM or less.

25. The peptide of claim 22, wherein the EC₅₀ is about 300 nM.

26. The peptide of claim 1, which comprises an amino acid sequence selected from the group consisting of SEQ ID NO: 43 and 91-102.

27. The peptide claim 1, wherein each of the amino acids is a D-amino acid.

28. The peptide claim 1, wherein each of the amino acids is a L-amino acid.

29. The peptide of claim 1, further comprising a dansyl moiety.

30. The peptide of claim 1, wherein the virus is a Flavivirus.

31. The peptide of claim 1, wherein the virus is a Hepatitis C virus, West Nile virus or the Dengue virus.

32. An isolated peptide having the amino acid sequence of any of SEQ ID NO: 4-86.

33. The peptide of claim 32, which has the amino acid sequence of any one of SEQ ID NO: 6, 8, 12, 13, 14, 21, 23, 24, 27, 28, 30, 32, 37, 44, 47, 48 and 53.

34. The peptide of claim 33, which has the amino acid sequence of SEQ ID NO: 6, 8, 12, 13, 14, 24, 27, 30, 32, 44, 48, and 53.

35. A pharmaceutical composition comprising (a) a peptide of 14 to 50 D- or L-amino acids in-length, wherein the peptide has an amphipathic α-helical structure, and wherein the peptide has anti-viral activity against a virus of the Flaviviridae family, and (b) a pharmaceutically acceptable carrier.

36. The pharmaceutical composition of claim 35, wherein the composition is a microbicide.

37. The pharmaceutical composition of claim 35, wherein the composition is a vaginal cream.

38. A pharmaceutical combination comprising (a) a peptide of 14 to 50 D- or L-amino acids in-length, wherein the peptide has an amphipathic α-helical structure, and wherein the peptide has anti-viral activity against a virus of the Flaviviridae family, and (b) an antiviral agent.

39. The pharmaceutical combination of claim 38, wherein the antiviral agent is α-interferon, pegylated interferon, ribavirin, amantadine, rimantadine, pleconaril, acyclovir, zidovudine, lamivudine, or a combination thereof.

40. A method for preventing viral infection in a mammalian cell comprising contacting the cell with an effective amount of a peptide of 14 to 50 D- or L-amino acids in-length, wherein the peptide has an amphipathic α-helical structure, and wherein the peptide has anti-viral activity against a virus of the Flaviviridae family.

41. The method of claim 40, wherein the mammalian cell is a human cell.

42. The method of claim 40, wherein the virus is a Flavivirus.

43. The method of claim 40, wherein the virus is Hepatitis C virus, West Nile virus or Dengue virus.

44. A method for preventing viral infection in a mammal comprising administering to the mammal an effective amount of a peptide of 14 to 50 D- or L-amino acids in-length, wherein the peptide has an amphipathic α-helical structure, and wherein the peptide has anti-viral activity against a virus of the Flaviviridae family; or administering to the mammal a pharmaceutical composition or combination comprising a peptide of 14 to 50 D- or L-amino acids in-length, wherein the peptide has an amphipathic α-helical structure, and wherein the peptide has anti-viral activity against a virus of the Flaviviridae family.

45. The method of claim 44, wherein the mammal is a human.

46. The method of claim 44, wherein the virus is a Flavivirus.

47. The method of claim 44, wherein the virus is Hepatitis C virus, West Nile virus or Dengue virus.

48. An article of manufacture comprising a vessel for collecting a body fluid and a peptide of 14 to 50 D- or L-amino acids in-length, wherein the peptide has an amphipathic α-helical structure, and wherein the peptide has anti-viral activity against a virus of the Flaviviridae family.

49. The article of claim 48, wherein the vessel is a collection bag, tube, capillary tube or syringe.

50. The article of claim 49, wherein the vessel is evacuated.

51. The article of claim 48, further comprising a biological stabilizer.

52. The article of claim 51, wherein the stabilizer is an anti-coagulant, preservative, protease inhibitor, or any combination thereof.

53. The article of claim 52, wherein the anti-coagulant is citrate, ethylene diamine tetraacetic acid, heparin, oxalate, fluoride or any combination thereof.

54. The article of claim 52, wherein the preservative is boric acid, sodium formate and sodium borate.

55. The article of claim 52, wherein the protease inhibitor is dipeptidyl peptidase IV.

56. The article of claim 55, wherein the protease inhibitor and/or stabilizer is freeze dried.

57. A composition comprising a sample from the body of a mammal and a peptide of 14 to 50 D- or L-amino acids in-length, wherein the peptide has an amphipathic α-helical structure, and wherein the peptide has anti-viral activity against a virus of the Flaviviridae family.

58. The composition of claim 57, further comprising a biological stabilizer.

59. The composition of claim 58, wherein the stabilizer is an anti-coagulant, a preservative, a protease inhibitor, or any combination thereof.

60. The composition of claim 59, wherein the anticoagulant is citrate, ethylene diamine tetraacetic acid, heparin, oxalate, fluoride or any combination thereof.

61. The composition of claim 59, wherein the preservative is boric acid, sodium formate and sodium borate.

62. The composition of claim 59, wherein the protease inhibitor is dipeptidyl peptidase IV.

63. The composition of claim 57, wherein the sample is a blood product.

64. The composition of claim 63, wherein the blood product is plasma, platelet, leukocytes or stem cell.
